(19) United States
(12) Patent Application Publication
(10) Pub. No.: US 2012/0010235 A1
(43) Pub. Date: Jan. 12, 2012

(54) N-SUBSTITUTED PYRROLIDINES

(76) Inventors: Xin-Jie Chu, Livingston, NJ (US); Qingjie Ding, Bridgewater, NJ (US); Nan Jiang, Pine Brook, NJ (US); Jin-Jun Liu, Warren, NJ (US); Tina Morgan Ross, Royersford, PA (US); Zhuming Zhang, Hillsborough, NJ (US)

(21) Appl. No.: 13/172,916

(22) Filed: Jun. 30, 2011

Related U.S. Application Data
(60) Provisional application No. 61/363,301, filed on Jul. 12, 2010.

(51) Int. Cl. A61K 31/437; A61K 31/40; C07D 487/04; A61K 31/407; C07D 401/12; A61K 31/454; C07D 401/14; C07D 403/12; A61K 31/4155; A61K 31/4196; A61K 31/4178; C07D 403/06; A61K 31/4025; C07D 471/04; A61P 35/00; C07D 207/16

(52) U.S. Cl. 514/299; 548/537; 514/423; 548/453; 514/413; 546/208; 514/326; 546/211; 548/364.1; 514/407; 548/265.4; 514/383; 548/314.7; 514/397; 548/524; 514/422; 546/112

(57) ABSTRACT

Compounds of formula

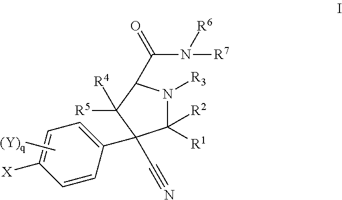

and enantiomers and pharmaceutically acceptable salts thereof are described, as well as the pharmaceutical compositions containing said compounds and their pharmaceutically acceptable salts, and the use of said compounds and pharmaceutical compositions for the treatment, control, amelioration or prevention of cancer.

### PRIORITY TO RELATED APPLICATIONS

This application claims the benefit of U.S. Provisional Application No. 61/363,301, filed Jul. 12, 2010, which is hereby incorporated by reference in its entirety.

### FIELD OF THE INVENTION

The present invention relates to pyrrolidine-2-carboxamide derivatives which act as inhibitors of MDM2-p53 interactions and are useful in the amelioration or treatment of cancer, especially solid tumors.

### BACKGROUND OF THE INVENTION

p53 is a tumor suppresser protein that plays a central role in protection against development of cancer. It guards cellular integrity and prevents the propagation of permanently damaged clones of cells by the induction of growth arrest or apoptosis. At the molecular level, p53 is a transcription factor that can activate a panel of genes implicated in the regulation of cell cycle and apoptosis. p53 is a potent cell cycle inhibitor which is tightly regulated by MDM2 at the cellular level. MDM2 and p53 form a feedback control loop. MDM2 can bind p53 and inhibit its ability to transactivate p53-regulated genes. In addition, MDM2 mediates the ubiquitin-dependent degradation of p53. p53 can activate the expression of the MDM2 gene, thus raising the cellular level of MDM2 protein. This feedback control loop insures that both MDM2 and p53 are kept at a low level in normal proliferating cells. MDM2 is also a cofactor for E2F, which plays a central role in cell cycle regulation.

The ratio of MDM2 to p53 (E2F) is dysregulated in many cancers. Frequently occurring molecular defects in the p16INK4/p19ARF locus, for instance, have been shown to affect MDM2 protein degradation. Inhibition of MDM2-p53 interaction in tumor cells with wild-type p53 should lead to accumulation of p53, cell cycle arrest and/or apoptosis. MDM2 antagonists, therefore, can offer a novel approach to cancer therapy as single agents or in combination with a broad spectrum of other antitumor therapies. The feasibility of this strategy has been shown by the use of different macromolecular tools for inhibition of MDM2-p53 interaction (e.g. antibodies, antisense oligonucleotides, peptides). MDM2 also binds E2F through a conserved binding region as p53 and activates E2F-dependent transcription of cyclin A, suggesting that MDM2 antagonists might also have effects in p53 mutant cells.

### SUMMARY OF THE INVENTION

One aspect of the invention is a compound of formula I

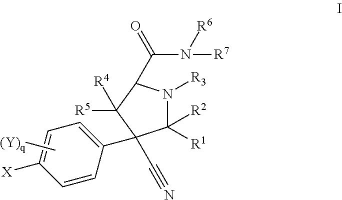

or a pharmaceutically acceptable salt thereof, wherein X, Y, R¹, R², R³, R⁴, R⁵, R⁶, R⁷ and q are as defined below.

The present invention also relates to pharmaceutical compositions comprising one or more compounds of the invention, or a pharmaceutically acceptable salt, and a pharmaceutically acceptable carrier or excipient.

The present invention further relates to a method of treating, ameliorating or preventing cancer in a mammal, preferably a human, comprising administering to said mammal a therapeutically effective amount of a compound according to the invention or a pharmaceutically acceptable salt thereof.

### DETAILED DESCRIPTION OF THE INVENTION

### Definitions

As used herein, the following terms shall have the following definitions.

The term “alkyl” refers to straight- or branched-chain saturated hydrocarbon groups having from 1 to about 12 carbon atoms, including groups having from 1 to about 7 carbon atoms. In certain embodiments, alkyl substituents may be lower alkyl substituents. The term “lower alkyl” refers to alkyl groups having from 1 to 6 carbon atoms, preferably from 1 to 4 carbon atoms. Examples of alkyl groups include, but are not limited to, methyl, ethyl, n-propyl, i-propyl, n-butyl, s-butyl, t-butyl, n-pentyl, and s-pentyl.

The term “alkenyl” as used herein means an unsaturated straight-chain or branched aliphatic hydrocarbon group containing at least one double bond and having 2 to 6, preferably 2 to 4 carbon atoms. Examples of such “alkenyl group” are vinyl, ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

“Alkoxy, alkoxyl or lower alkoxy” refers to any of the above lower alkyl groups which is attached to the remainder of the molecule by an oxygen atom (RO—). Typical lower alkoxy groups include methoxy, ethoxy, isopropoxy or propoxy, butyloxy and the like. Further included within the meaning of alkoxy are multiple alkoxy side chains, e.g. ethoxy ethoxy, methoxy ethoxy, methoxy ethoxy ethoxy and the like and substituted alkoxy side chains, e.g., dimethylamino ethoxy, diethylamino ethoxy, dimethoxy-phosphoryl methoxy and the like.

The term “alkynyl” as used herein means an unsaturated straight-chain or branched aliphatic hydrocarbon group containing one triple bond and having 2 to 6, preferably 2 to 4 carbon atoms. Examples of such “alkynyl group” are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

Amino means the group —NH₂.

“Aryl” means a monovalent, monocyclic or bicyclic, aromatic carboxylic hydrocarbon radical, preferably a 6-10 member aromatic ring system. Preferred aryl groups include, but are not limited to, phenyl, naphthyl, tolyl, and xylyl.

Carboxyl or carboxy means the monovalent group —COOH. Carboxy lower alkyl means —COOR, wherein R is lower alkyl. Carboxy lower alkoxy means —COOROH wherein the R is lower alkyl.

Carbonyl means the group

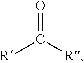

where R′ and R″ independently can be any of a number of chemical groups including alkyl.

The term “cycloalkyl” as used herein means any stable monocyclic or polycyclic system which consists of carbon atoms only, any ring of which being saturated, and the term “cycloalkenyl” is intended to refer to any stable monocyclic or polycyclic system which consists of carbon atoms only, with at least one ring thereof being partially unsaturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds. Examples of cycloalkenyls include, but are not limited to, cyclopentenyl or cyclohexenyl.

The term “halogen” as used herein means fluorine, chlorine, bromine, or iodine, preferably fluorine and chlorine.

“Heteroaryl” means an aromatic heterocyclic ring system containing up to two rings. Preferred heteroaryl groups include, but are not limited to, thienyl, furyl, indolyl, pyrrolyl, pyridinyl, pyrazinyl, oxazolyl, thiaxolyl, quinolinyl, pyrimidinyl, imidazole substituted or unsubstituted triazolyl and substituted or unsubstituted tetrazolyl.

In the case of aryl or heteroaryl which are bicyclic it should be understood that one ring may be aryl while the other is heteroaryl and both being substituted or unsubstituted.

“Hetero atom” means an atom selected from N, O and S.

“Heterocycle” or “heterocyclic ring” means a substituted or unsubstituted 5 to 8 membered, mono- or bicyclic, non-aromatic hydrocarbon, wherein 1 to 3 carbon atoms are replaced by a hetero atom selected from nitrogen, oxygen or sulfur atom. Examples include pyrrolidin-2-yl; pyrrolidin-3-yl; piperidinyl; morpholin-4-yl and the like which in turn can be substituted.

Hydroxy or hydroxyl is a prefix indicating the presence of a monovalent —O—H group.

“IC50” refers to the concentration of a particular compound required to inhibit 50% of a specific measured activity. IC₅₀ can be measured, inter alia, as is described subsequently in Example 72.

“Lower” as in “lower alkenyl” means a group having 1 to 6 carbon atoms.

“Nitro” means —NO₂.

Oxo means the group ═O.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoro acetic acid and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (1995) at pgs. 456-457.

“Substituted,” as in substituted alkyl, means that the substitution can occur at one or more positions and, unless otherwise indicated, that the substituents at each substitution site are independently selected from the specified options. The term “optionally substituted” refers to the fact that one or more hydrogen atoms of a chemical group (with one or more hydrogen atoms) can be, but does not necessarily have to be, substituted with another substituent.

In one embodiment, the present invention relates to compounds of formula I

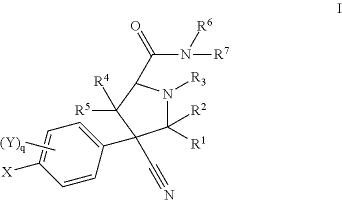

wherein
X is selected from the group consisting of H, F, Cl, Br, I, cyano, nitro, lower alkynyl, cycloalkyl, lower alkyl, lower alkenyl and lower alkoxy;
Each Y is independently selected from the group consisting of H, F, Cl, Br, I, CN, OH, nitro, lower alkyl, cycloalkyl, lower alkoxy, lower alkenyl, cycloalkenyl, lower alkynyl, aryl, heteroaryl, heterocycle, COOR′, OCOR′, CONR′R″, NR′COR″, NR″SO₂R′, SO₂NR′R″ and NR′R″, wherein R′ and R″ are independently selected from

H,

lower alkyl optionally substituted with halogen, hydroxy, NH₂, NH-lower alkyl, N(lower-alkyl)₂, oxo, carboxy, carboxy lower alkyl and cycloalkyl, heteroaryl,

lower cycloalkyl optionally substituted with halogen, lower alkoxy, lower alkyl, carboxy, carboxy lower alkoxy, carboxy lower alkyl, oxo, CN, and NHSO₂-lower alkyl,

lower alkenyl optionally substituted with halogen, hydroxy, NH₂ and NH-lower alkyl and N(lower-alkyl)₂,

lower alkynyl optionally substituted with halogen, hydroxy, NH₂ and NH-lower alkyl and N(lower-alkyl)₂,

lower cycloalkenyl optionally substituted with halogen, lower alkoxy, lower alkyl, carboxy, carboxy lower alkoxy, oxo and CN,

aryl optionally substituted with halogen, lower alkoxy, hydroxy, lower alkyl, lower alkenyl, cycloalkyl, carboxy, carboxy lower alkoxy, carboxy lower alkyl, oxo, R¹ and CN, CONR¹R²

hetereoaryl optionally substituted with halogen, lower alkoxy, lower alkyl, carbonyl, carboxy, carboxy lower alkyl, carboxy lower alkoxy, oxo, R¹ and CN, and

hetereocycle optionally substituted with halogen, lower alkoxy, carboxy, carboxy lower alkoxy, oxo CN, R¹, SO₂-lower alkyl and lower alkyl that is optionally substituted with oxo,

or alternatively, when Y is CONR′R″ or SO₂NR′R″, R′ and R″ together with the N to which they are attached can form a ring having 3 to 7 atoms, said ring being selected from or cycloalkyl, cycloalkenyl heteroaryl and or heterocycle, said ring optionally being substituted with halogen, lower alkoxy, lower alkyl, carboxy, carboxy lower alkoxy, oxo and CN;
one of R¹ and R² is H and the other is selected from the group consisting of

lower alkyl optionally substituted with halogen, hydroxy, NH₂, NH-lower alkyl and N(lower-alkyl)₂,

lower alkenyl optionally substituted with halogen, hydroxy, NH₂, NH-lower alkyl and N(lower-alkyl)₂,

aryl optionally substituted with halogen, lower alkoxy, lower alkyl, carboxy, carboxy lower alkoxy, oxo and CN,

heteroaryl optionally substituted with halogen, lower alkoxy, lower alkyl, carboxy, carboxy lower alkoxy, oxo and CN,

heterocycle optionally substituted with R′, halogen, lower alkoxy, carboxy, carboxy lower alkoxy, oxo, CN, SO₂—R′,

cycloalkyl and cycloalkyl substituted with halogen, lower alkoxy, lower alkyl, hydroxycarbony, carboxy, carboxy lower alkoxy, oxo and CN, and

cycloalkenyl and cycloalkenyl substituted with halogen, lower alkoxy, lower alkyl, hydroxycarbony, carboxy, carboxy lower alkoxy, oxo and CN;

R³ is selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, wherein R′ and R″ are as defined above for Y,
or alternatively, R³ together with R¹ or R² can form a ring having 3 to 7 atoms, said ring being selected from heteroaryl and heterocycle, said ring optionally being substituted with halogen, lower alkoxy, lower alkyl, hydroxycarbony, carboxy, carboxy lower alkoxy, oxo and CN;
one of R⁴ and R⁵ is selected from the group consisting of hydrogen, lower alkyl and CF₃ and the other is selected from the group consisting of

lower alkyl optionally substituted with halogen, hydroxy, NH₂, NH-lower alkyl and N(lower-alkyl)₂,

lower alkenyl optionally substituted with halogen, hydroxy, NH₂, NH-lower alkyl and N(lower-alkyl)₂,

aryl optionally substituted with halogen, lower alkoxy, lower alkyl, hydroxycarbony, carboxy, carboxy lower alkoxy, oxo and CN,

heteroaryl optionally substituted with halogen, lower alkoxy, lower alkyl, carboxy, hydroxyl, carboxy lower alkoxy, oxo, nitro and CN,

heterocycle optionally substituted with halogen, lower alkoxy, lower alkyl, hydroxycarbony, carboxy, carboxy lower alkoxy, oxo and CN,

cycloalkyl optionally substituted with halogen, lower alkoxy, lower alkyl, hydroxycarbony, carboxy, carboxy lower alkoxy, oxo and CN, and

cycloalkenyl optionally substituted with halogen, lower alkoxy, lower alkyl, hydroxycarbony, carboxy, carboxy lower alkoxy, oxo and CN;

R⁶ and R⁷ are each independently selected from the group consisting of H, (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ(CH₂)ₙ—NR′SO₂R″, (CH₂CH₂O)ₘ(CH₂)ₙ—COOH, (CH₂CH₂O)ₘ(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, —COR′, —SOR′, SO₂R′, Aryl-(CH₂)ₙ—COOH, heteroaryl-lower alkyl-CO-lower alkyl-NR′R″ wherein the lower alkyl may be substituted with OR′, and heteroaryl-(CH₂)ₙ-heterocycle wherein the heterocycle may optionally be substituted with lower alkyl, hydroxyl, COOR′ and COR′;
wherein R′ and R″ are as defined above for Y;
m, n and p are independently 0 to 6; and
q is 1 to 4;
or a pharmaceutically acceptable salt thereof.

In another embodiment, the invention relates to compounds of formula I having the following stereochemical orientation

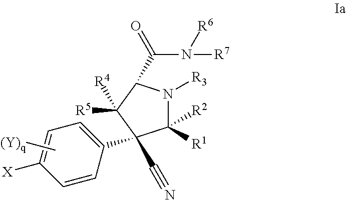

wherein X, Y, R¹, R², R³, R⁴, R⁵, R⁶, R⁷ m, n, and q are as defined above, or a pharmaceutically acceptable salt thereof.

Another embodiment of the invention relates to compounds of Formula I, including compounds of Formula Ia, or a pharmaceutically acceptable salt thereof wherein X is selected from the group consisting of H, F, Cl, Br, I, cyano, nitro, ethynyl, cyclopropyl, methyl, ethyl, isopropyl, vinyl and methoxy.

Another embodiment of the invention relates to compounds of Formula I, including compounds of Formula Ia, or a pharmaceutically acceptable salt thereof wherein each Y is independently selected from the group consisting of H, F, Cl, Br, I, CN, OH, nitro, lower alkyl, cycloalkyl, lower alkoxy, lower alkenyl, lower cycloalkenyl and lower alkynyl, and q is 1 or 2.

Another embodiment of the invention relates to compounds of Formula I, including compounds of Formula Ia, or a pharmaceutically acceptable salt thereof wherein

one of R¹ and R² is H and the other is selected from the group consisting of
lower alkyl optionally substituted with OH or halogen, and
cycloalkyl optionally substituted with halogen, lower alkoxy, lower alkyl and carboxy.

Another embodiment of the invention relates to compounds of Formula I, including compounds of Formula Ia, or a pharmaceutically acceptable salt thereof wherein R³ is selected from the group consisting of (CH₂)ₙ—R′, (CH₂)ₙ—COOR′, (CH₂)ₙ—CONR′R″, (CH₂)ₙ—OR′, (CH₂)ₙ—COR′, and R′ and R″ are each independently selected from
H,
lower alkyl, optionally substituted by hydroxy and amino,
aryl optionally substituted with lower alkyl, lower alkoxy, halogen and carboxy lower alkyl.

Another embodiment of the invention relates to compounds of Formula I, including compounds of Formula Ia, or a pharmaceutically acceptable salt thereof wherein R⁴ is H and R⁵ is selected from the group consisting of aryl, aryl substituted with Cl or F, and heteroaryl optionally substituted with H, F, Cl, Br, I, CN, OH, nitro and lower alkyl.

Another embodiment of the invention relates to compounds of Formula I, including compounds of Formula Ia, or a pharmaceutically acceptable salt thereof wherein R⁶ and R⁷ are independently selected from the group consisting of H, (CH₂)ₙ—R′, (CH₂)ₙ—NR′R″, (CH₂)ₙ—NR′COR″, (CH₂)ₙ—COOH, (CH₂)ₙ—COOR′, (CH₂)ₙ—OR′, (CH₂)ₙ—COR′, —COR′, —SOR′, SO₂R′, Aryl-COOR′, heteroaryl-lower alkyl-CO-lower alkyl-NR′R″ wherein the lower alkyl optionally is substituted with OR′, and R⁶ or R⁷ may be heteroaryl-(CH₂)ₙ-heterocycle wherein the heterocycle optionally may be substituted with lower alkyl, hydroxy, COOR′ and COR′.

Another embodiment of the invention relates to compounds of Formula 1, including compounds of Formula Ia, or a pharmaceutically acceptable salt thereof, wherein R′ and R″ are independently selected from
H,
lower alkyl optionally substituted by the group consisting of hydroxyl and amino, and
aryl optionally substituted with lower alkyl, lower alkoyl, halogen and carboxy-lower alkyl.

Another embodiment of the invention relates to compounds of Formula I, including compounds of Formula Ia, or a pharmaceutically acceptable salt thereof, wherein m, n and p are 1 or 0 and q is 1.

Another embodiment of the invention relates to compounds of Formula Ia wherein

X is selected from the group consisting of H, F, Cl, Br, I, cyano, nitro, ethynyl, cyclopropyl, methyl, ethyl, isopropyl, vinyl and methoxy;
each Y is independently selected from the group consisting of H, F, Cl, Br, I, CN, OH, nitro, lower alkyl, cycloalkyl, lower alkoxy, lower alkenyl, lower cycloalkenyl and lower alkynyl;
one of R¹ and R² is H and the other is selected from the group consisting of
lower alkyl optionally substituted with OH or halogen, and
cycloalkyl optionally substituted with halogen, lower alkoxy, lower alkyl and carboxy;
R³ is selected from the group consisting of (CH₂)ₙ—R′, (CH₂)ₙ—COOR′, (CH₂)ₙ—CONR′R″, (CH₂)ₙ—OR′, (CH₂)ₙ—COR′,
or alternatively, R³ together with R¹ form a heterocycle that is optionally substituted with the group consisting of oxo and lower alkyl;
one of R⁴ and R⁵ is H and the other is selected from the group consisting of aryl, aryl substituted with Cl or F, and heteroaryl optionally substituted with H, F, Cl, Br, I, CN, OH, nitro and lower alkyl;
R⁶ and R⁷ are independently selected from the group consisting of H, (CH₂)ₙ—R′, (CH₂)ₙ—NR′R″, (CH₂)ₙ—NR′COR″, (CH₂)ₙ—COOH, (CH₂)ₙ—COOR′, (CH₂)ₙ—OR′, (CH₂)ₙ—COR′, —COR′, —SOR′ and SO₂R′, Aryl-COOR′—, heteroaryl-lower alkyl-CO-lower alkyl-NR′R″ wherein the lower alkyl optionally is substituted with OR′, and R⁶ or R⁷ may be heteroaryl-(CH₂)ₙ-heterocycle wherein the heterocycle optionally may be substituted with lower alkyl, hydroxy, COOR′ and COR′;
R′ and R″ are as defined above; and
m, n and p are 0 or 1 and q is 1;
or a pharmaceutically acceptable salt thereof.

Another embodiment of the invention relates to compounds of Formula Ia wherein

X is selected from the group consisting of F, Cl and Br;
Y is selected from the group consisting of H and F;
R¹ is lower alkyl;

### R² is H;

R³ is selected from the group consisting of (CH₂)ₙ—R′, (CH₂)ₙ—COR′, (CH₂)ₙ—OR′ and (CH₂)ₙCONR′R″,
or alternatively, R³ together with R¹ form a heterocycle that is optionally substituted with the group consisting of oxo and lower alkyl;

### R⁴ is H;

R⁵ is aryl optionally substituted with Cl or F;

### R⁶ is H;

R⁷ is selected from the group

lower alkyl optionally substituted with cycloalkyl, heteroaryl, COOR′ and OR′,

heterocycle optionally substituted with R′, COR′ SO₂R′,

heteroaryl-lower alkyl-CO-lower alkyl-NR′R″, wherein the lower alkyl optionally is substituted with OR′,

aryl optionally substituted with R′, COOR′, OR′, halogen and CONR′R″, and

heteroaryl optionally substituted with OR′, COR′, R′, CO₂R′ and (CH₂)ₙ-heterocycle wherein the heterocycle optionally is substituted with OR′, COOR′ or COR′;

R′ is selected from the group consisting of

H,

lower alkyl optionally substituted with hydroxyl, carboxy, carboxy lower alkyl, cycloalkyl and heteroaryl,

lower cycloalkyl optionally substituted with carboxy-lower alkyl and NHSO₂-lower alkyl,

lower alkenyl,

lower alkynyl optionally substituted with halogen, hydroxyl, NH₂, NH-lower alkyl and N(lower alkyl)₂,

aryl optionally substituted with lower alkyl, lower alkoxy, F, Cl, lower alkyenyl, cycloalkyl, carboxy, carboxy lower alkyl and CONR′R²,

heteroaryl optionally substituted with carboxy, carboxy-lower alkyl and R′,

heterocycle optionally substituted with lower alkoxy, R′, SO₂-lower alkyl and lower alkyl wherein the lower alkyl may be substituted with oxo;

m and n are independently 0 or 1; and
q is 1;
or a pharmaceutically acceptable salt thereof.

Another embodiment of the invention relates to compounds of Formula I, including compounds of Formula Ia, wherein R¹ is a substituted lower alkyl selected from:

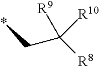

where R⁸ and R⁹ are both methyl, or alternatively, R⁸ and R⁹ together with the carbon to which they are attached form a ring selected from cyclopropyl, cyclobutyl, cyclopentyl and acyclohexyl;
R¹⁰ is (CH₂)ₘ—R¹¹, where m is 0, 1 or 2;
R¹¹ is selected from the group consisting of hydrogen, hydroxyl, lower alkyl, lower alkoxy, aryl, hetereoaryl, and hetereocycle;

### R² is H;

R³ is selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, wherein R′ and R″ are as defined above;

### R⁴ is H;

R⁵ is a substituted phenyl selected from

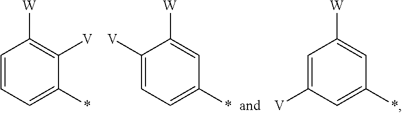

### W is F, Cl or Br;

### V is H or F;

one of R₆ and R₇ is hydrogen and the other is (CH₂)ₙ—R′;
n is 0 or 1; and
R′ is selected from the group consisting of

H,

lower alkyl optionally substituted with hydroxyl, carboxy, carboxy lower alkyl, cycloalkyl and heteroaryl,

lower cycloalkyl optionally substituted with carboxy-lower alkyl and NHSO₂-lower alkyl,

lower alkenyl,

lower alkynyl optionally substituted with halogen, hydroxyl, NH₂, NH-lower alkyl and N(lower alkyl)₂,

aryl optionally substituted with lower alkyl, lower alkoxy, F, Cl, lower alkyenyl, cycloalkyl, carboxy, carboxy lower alkyl and CONR¹R²,

heteroaryl optionally substituted with carboxy, carboxy-lower alkyl and R′,

heterocycle optionally substituted with COR¹, SO₂R¹, R′, SO₂-lower alkyl and lower alkyl wherein the lower alkyl may be substituted with oxo;

or a pharmaceutically acceptable salt thereof.

Compounds according to the invention include:
rac-(2R,3R,4R,5S)-3-(3-chloro-phenyl)-4-(4-chloro-phenyl)-4-cyano-5-(2,2-dimethyl-propyl)-1-ethyl-pyrrolidine-2-carboxylic acid;
rac-(2R,3S,4R,5S)-3-(3-chloro-2-fluoro-phenyl)-4-(4-chloro-2-fluoro-phenyl)-4-cyano-5-methyl-1-propionyl-pyrrolidine-2-carboxylic acid (3,4-dihydroxy-butyl)-amide,
rac-(2R,3S,4R,5S)-3-(3-chloro-2-fluoro-phenyl)-4-(4-chloro-2-fluoro-phenyl)-4-cyano-1-(2-ethoxy-benzoyl)-5-methyl-pyrrolidine-2-carboxylic acid (3,4-dihydroxy-butyl)-amide,
rac-(2R,3S,4R,5S)-3-(3-chloro-2-fluoro-phenyl)-4-(4-chloro-2-fluoro-phenyl)-4-cyano-1-(3-ethoxy-benzoyl)-5-methyl-pyrrolidine-2-carboxylic acid (3,4-dihydroxy-butyl)-amide,
rac-(2R,3S,4R,5S)-3-(3-chloro-2-fluoro-phenyl)-4-(4-chloro-2-fluoro-phenyl)-4-cyano-1-(3-methyl-benzoyl)-5-methyl-pyrrolidine-2-carboxylic acid (3,4-dihydroxy-butyl)-amide
rac-(1R,2S,3R,7aS)-1-(4-chloro-2-fluoro-phenyl)-2-(3-chloro-2-fluoro-phenyl)-1-cyano-6,6-dimethyl-5-oxo-hexahydro-pyrrolizine-3-carboxylic acid ((S)-3,4-dihydroxy-butyl)-amide,
rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid ((S)-3,4-dihydroxy-butyl)-amide,
rac-(1R,2S,3R,7aS)-1-(4-chloro-2-fluoro-phenyl)-2-(3-chloro-2-fluoro-phenyl)-1-cyano-6,6-dimethyl-hexahydro-pyrrolizine-3-carboxylic acid ((S)-3,4-dihydroxy-butyl)-amide,
rac-(2R,3S,4R,5S)-3-(3-chloro-2-fluoro-phenyl)-4-(4-chloro-2-fluoro-phenyl)-4-cyano-1-(2-methyl-benzoyl)-5-methyl-pyrrolidine-2-carboxylic acid (3,4-dihydroxy-butyl)-amide,
rac-(2R,3S,4R,5R)-1-acetyl-3-(3-chloro-2-fluoro-phenyl)-4-(4-chloro-2-fluoro-phenyl)-4-cyano-5-methyl-pyrrolidine-2-carboxylic acid (1-acetyl-piperidin-4-yl)-amide,
rac-(2R,3S,4R,5S)-1-acetyl-3-(3-chloro-2-fluoro-phenyl)-4-(4-chloro-2-fluoro-phenyl)-4-cyano-5-methyl-pyrrolidine-2-carboxylic acid (1-acetyl-piperidin-4-yl)-amide,
rac-(2R,3S,4R)-3-(3-chloro-2-fluoro-phenyl)-4-(4-chloro-2-fluoro-phenyl)-4-cyano-1-(3-methyl-benzyl)-5-methyl-pyrrolidine-2-carboxylic acid (1-acetyl-piperidin-4-yl)-amide,
rac-(2R,3S,4R)-3-(3-chloro-2-fluoro-phenyl)-4-(4-chloro-2-fluoro-phenyl)-4-cyano-5-methyl-1-(3-methyl-benzyl)-pyrrolidine-2-carboxylic acid (1-methanesulfonyl-piperidin-4-yl)-amide,
rac-(2R,3S,4R)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid (1-acetyl-piperidin-4-yl)-amide
rac-(2R,3S,4R,5S)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid (1-acetyl-piperidin-4-yl)-amide,
rac-(2R,3S,4R,5R)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid (1-acetyl-piperidin-4-yl)-amide,
rac-(1R,2S,3R,8aS)-2-(3-chloro-2-fluoro-phenyl)-1-(4-chloro-2-fluoro-phenyl)-1-cyano-7,7-dimethyl-octahydro-indolizine-3-carboxylic acid ((S)-3,4-dihydroxy-butyl)-amide,
rac-(2R,3S,4R,5R)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid (1-methanesulfonyl-piperidin-4-yl)-amide,
rac-(2R,3S,4R,5S)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid (1-methanesulfonyl-piperidin-4-yl)-amide,
(2R,3S,4R)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid (4-methanesulfonylamino-cyclohexyl)-amide,
rac-(2R,3S,4R)-3-(3-chloro-2-fluoro-phenyl)-4-(4-chloro-2-fluoro-phenyl)-4-cyano-1-(3-methoxy-benzyl)-5-methyl-pyrrolidine-2-carboxylic acid (1-acetyl-piperidin-4-yl)-amide,
rac-(2R,3S,4R)-3-(3-chloro-2-fluoro-phenyl)-4-(4-chloro-2-fluoro-phenyl)-4-cyano-1-(2-chloro-benzyl)-5-methyl-pyrrolidine-2-carboxylic acid (1-acetyl-piperidin-4-yl)-amide,
rac-(2R,3S,4R,5S)-1-butyl-3-(3-chloro-2-fluoro-phenyl)-4-(4-chloro-2-fluoro-phenyl)-4-cyano-5-(2,2-dimethyl-propyl)-pyrrolidine-2-carboxylic acid [1-(1-butyl-4-hydroxy-piperidin-4-ylmethyl)-1H-pyrazol-3-yl]-amide,
rac-propionic acid 4-(3-{[(2R,3S,4R,5S)-3-(3-chloro-2-fluoro-phenyl)-4-(4-chloro-2-fluoro-phenyl)-4-cyano-5-(2,2-dimethyl-propyl)-1-propionyl-pyrrolidine-2-carbonyl]-amino}-pyrazol-1-ylmethyl)-1-propionyl-piperidin-4-yl ester,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-benzoic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-fluoro-benzoic acid trifluoroacetate salt,
chiral 2-(4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]amino}-phenyl)-2-methyl-propionic acid,
rac 4-({(2R,3S,4R,5S)-3-(3-chloro-2-fluoro-phenyl)-4-(4-chloro-2-fluoro-phenyl)-4-cyano-5-(2,2-dimethyl-propyl)-1-[2-(2-fluoro-phenyl)-ethyl]-pyrrolidine-2-carbonyl}-amino)-benzoic acid methyl ester,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-benzoic acid methyl ester,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-propyl-pyrrolidine-2-carbonyl]-amino}-benzoic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-isobutyl-pyrrolidine-2-carbonyl]-amino}-benzoic acid,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}benzoic acid,
rac-4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-benzoic acid,
rac-4-{[(2R,3S,4R,5S)-3-(3-chloro-2-fluoro-phenyl)-4-(4-chloro-2-fluoro-phenyl)-4-cyano-1-cyclobutylmethyl-5-(2,2-dimethyl-propyl)-pyrrolidine-2-carbonyl]-amino}-benzoic acid,
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-benzoic acid,
rac-4-{[(2R,3S,4R,5S)-3-(3-chloro-2-fluoro-phenyl)-4-(4-chloro-2-fluoro-phenyl)-4-cyano-1-(2-cyclohexyl-ethyl)-5-(2,2-dimethyl-propyl)-pyrrolidine-2-carbonyl]-amino}-benzoic acid,
chiral 4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-2-methoxy-benzoic acid,
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid,
rac-4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-3-methoxy-benzoic acid,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-2-methoxy-benzoic acid,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-methoxy-benzoic acid,
rac 4-{[(2R,3S,4R,5S)-1-butyl-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid,
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-3-methoxy-benzoic acid,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(3-methyl-butyl)-pyrrolidine-2-carbonyl]-amino}-2-methoxy-benzoic acid,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-pentyl-pyrrolidine-2-carbonyl]-amino}-2-methoxy-benzoic acid,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-pent-4-enyl-pyrrolidine-2-carbonyl]-amino}-2-methoxy-benzoic acid,
rac-4-{[(2R,3S,4R,5S)-3-(3-chloro-2-fluoro-phenyl)-4-(4-chloro-2-fluoro-phenyl)-4-cyano-1-(2-cyclohexyl-ethyl)-5-(2,2-dimethyl-propyl)-pyrrolidine-2-carbonyl]-amino}-2-methoxy-benzoic acid trifluoroacetate salt,
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-propyl-pyrrolidine-2-carbonyl]-amino}-2-methoxy-benzoic acid,
rac-(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (4-carbamoyl-2-methoxy-phenyl)-amide,
rac-(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (2-methoxy-4-methylcarbamoyl-phenyl)-amide,
rac-(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carboxylic acid {1-[2-((R)-3-dimethylamino-2-hydroxy-propoxy)-2-methyl-propyl]-1H-pyrazol-3-yl}-amide,
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-(5-hydroxy-pentyl)-pyrrolidine-2-carbonyl]-amino}-benzoic acid,
chiral 5-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2H-[1,2,4]triazole-3-carboxylic acid methyl ester,
chiral 2-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3H-imidazole-4-carboxylic acid trifluoracetate salt,
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-(5-hydroxy-pentyl)-pyrrolidine-2-carbonyl]-amino}-3-methoxy-benzoic acid,
chiral (2R,3S,4R,5S)-4-(4-chloro-2-fluoro-phenyl)-3-(3-chloro-2-fluoro-phenyl)-4-cyano-1-cyclopropylmethyl-5-(2,2-dimethyl-propyl)-pyrrolidine-2-carboxylic acid ethylamide,
chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid [1-(2-hydroxy-2-methyl-propyl)-1H-pyrazol-3-yl]-amide,
(R)-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-cyclopropyl-acetic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(2-ethoxycarbonyl-cyclopropylmethyl)-pyrrolidine-2-carbonyl]-amino}-3-methoxy-benzoic acid methyl ester,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-prop-2-ynyl-pyrrolidine-2-carbonyl]-amino}-3-methoxy-benzoic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(ethoxycarbonylmethyl-carbamoyl)-pyrrolidine-2-carbonyl]-amino}-3-methoxy-benzoic acid,
chiral (2S,3R,4S,5R)-4-(3-chloro-2-fluoro-phenyl)-3-(4-chloro-2-fluoro-phenyl)-2-(2,2-dimethyl-propyl)-5-((R)-2-hydroxymethyl-pyrrolidine-1-carbonyl)-1-methyl-pyrrolidine-3-carbonitrile,
chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid [(S)-2-hydroxy-1-(1H-imidazol-4-ylmethyl)-ethyl]-amide trifluoroacetate salt,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-1-ethyl-1H-pyrrole-2-carboxylic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1S,2S)-2-methoxycarbonyl-cyclopropylmethyl)-pyrrolidine-2-carbonyl]-amino}-3-methoxy-benzoic acid methyl ester,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1R,2R)-2-methoxycarbonyl-cyclopropylmethyl)-pyrrolidine-2-carbonyl]-amino}-3-methoxy-benzoic acid methyl ester,
4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1S,2S)-2-ethoxycarbonyl-cyclopropylmethyl)-pyrrolidine-2-carbonyl]-amino}-3-methoxy-benzoic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1R,2R)-2-ethoxycarbonyl-cyclopropylmethyl)-pyrrolidine-2-carbonyl]-amino}-3-methoxy-benzoic acid, and
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-(5-hydroxy-pentyl)-pyrrolidine-2-carbonyl]-amino}-2-methoxy-benzoic acid,
   and the pharmaceutically acceptable salts of the foregoing compounds.

The following compounds also comprise an embodiment of the invention:
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-benzoic acid;
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-fluoro-benzoic acid trifluoroacetate salt;
chiral 2-(4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]amino}-phenyl)-2-methyl-propionic acid;
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid;
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-isobutyl-pyrrolidine-2-carbonyl]-amino}-benzoic acid;
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}benzoic acid;
rac-4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-benzoic acid;
rac-4-{[(2R,3S,4R,5S)-3-(3-chloro-2-fluoro-phenyl)-4-(4-chloro-2-fluoro-phenyl)-4-cyano-1-(2-cyclohexyl-ethyl)-5-(2,2-dimethyl-propyl)-pyrrolidine-2-carbonyl]-amino}-benzoic acid;
chiral 4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-2-methoxy-benzoic acid;
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid;
rac-4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-3-methoxy-benzoic acid;
racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-2-methoxy-benzoic acid;
racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-methoxy-benzoic acid;
racemic 4-{[(2R,3S,4R,5S)-1-butyl-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid;
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-3-methoxy-benzoic acid;
racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(3-methyl-butyl)-pyrrolidine-2-carbonyl]-amino}-2-methoxy-benzoic acid;
racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-pent-4-enyl-pyrrolidine-2-carbonyl]-amino}-2-methoxy-benzoic acid;
racemic 4-{[(2R,3S,4R,5S)-3-(3-chloro-2-fluoro-phenyl)-4-(4-chloro-2-fluoro-phenyl)-4-cyano-1-(2-cyclohexyl-ethyl)-5-(2,2-dimethyl-propyl)-pyrrolidine-2-carbonyl]-amino}-2-methoxy-benzoic acid trifluoroacetate salt;
racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-propyl-pyrrolidine-2-carbonyl]-amino}-2-methoxy-benzoic acid;
rac-(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (4-carbamoyl-2-methoxy-phenyl)-amide;
rac-(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (2-methoxy-4-methylcarbamoyl-phenyl)-amide;
chiral 5-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2H-[1,2,4]triazole-3-carboxylic acid methyl ester;
chiral 2-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3H-imidazole-4-carboxylic acid trifluoracetate salt;
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-(5-hydroxy-pentyl)-pyrrolidine-2-carbonyl]-amino}-3-methoxy-benzoic acid;
chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid [1-(2-hydroxy-2-methyl-propyl)-1-pyrazol-3-yl]-amide;
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(2-ethoxycarbonyl-cyclopropylmethyl)-pyrrolidine-2-carbonyl]-amino}-3-methoxy-benzoic acid methyl ester; and
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-prop-2-ynyl-pyrrolidine-2-carbonyl]-amino}-3-methoxy-benzoic acid,

and the pharmaceutically acceptable salts of the foregoing compounds.

The compounds of formula I, including compounds of formula Ia, as well as their salts that have at least one asymmetric carbon atom may be present as racemic mixtures or different stereoisomers. The various isomers can be isolated by known separation methods, e.g., chromatography.

Compounds disclosed herein and covered by formula I, including compounds of formula Ia, above may exhibit tautomerism or structural isomerism. It is intended that the invention encompasses any tautomeric or structural isomeric form of these compounds, or mixtures of such forms, and is not limited to any one tautomeric or structural isomeric form depicted in the formulas above.

### Dosages

The compounds of the present invention are inhibitors of MDM2-p53 interactions and are thus useful in the treatment or control of cell proliferative disorders, in particular chemoprevention of cancer. Chemoprevention is defined as inhibiting the development of invasive cancer by either blocking the initiating mutagenic event or by blocking the progression of pre-malignant cells that have already suffered an insult of inhibiting tumor relapse. These compounds and formulations containing said compounds are anticipated to be particularly useful in the treatment or control of solid tumors, such as, for example, breast, colon, lung and prostate tumors.

A “therapeutically effective amount” or “effective amount” of a compound in accordance with this invention means an amount of compound that is effective to prevent, alleviate or ameliorate symptoms of disease or prolong the survival of the subject being treated.

The therapeutically effective amount or dosage of a compound according to this invention can vary within wide limits. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration; it may be given as continuous infusion.

### Compositions/Formulations

In an alternative embodiment, the present invention includes pharmaceutical compositions comprising at least one compound of formula I, or a pharmaceutically acceptable salt thereof and a pharmaceutically acceptable excipient and/or carrier.

These pharmaceutical compositions can be suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a formula I compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

The pharmaceutical preparations of the invention can also contain preserving agents, solubilizing agents, stabilizing agents, wetting agents, emulsifying agents, sweetening agents, coloring agents, flavoring agents, salts for varying the osmotic pressure, buffers, coating agents or antioxidants. They can also contain other therapeutically valuable substances, including additional active ingredients other than those of formula I.

### General Synthesis of the Compounds According to the Invention

The present invention provides methods for the synthesis of the N-substituted pyrrolidine-2-carboxamides of the invention.

The compounds of the invention can be prepared by processes known in the art. Suitable processes for synthesizing these compounds are also provided in the examples. Generally, compounds of formula I can be synthesized according to one of the below described synthetic routes.

The key transformations are a convergent [2+3] cycloaddition of imine II and activated olefin III to generate pyrrolidine-3-carbonitrile compounds IV in a stereoselective and efficient manner, followed by a reductive amination with appropriate aldehydes.

The starting materials are either commercially available or can be synthesized by methods known to those of ordinary skill in the art. Preparations of intermediates II and III are illustrated in Schemes 1 and 2 below. In general an appropriately selected aldehyde or ketone can be reacted with glycine tert-butyl ester or glycine methyl ester to generate imine II as a crude product (see Scheme 1 below).

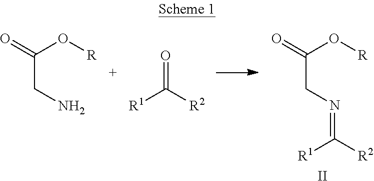

Reagents and conditions: R is tert-butyl or methyl

(1) If R¹ or R² is H, use CH₂Cl₂, room temperature, overnight;

(2) If R¹ and R² are both not H, use ethanol, 100° C., 48 h;

An intermediate of formula III can be made from a base-catalyzed condensation reaction of appropriately selected substituted-phenyl acetonitriles and aldehydes. The reaction proceeds in a highly stereoselective manner with the Z-isomer as the major or exclusive product (see scheme 2 below).

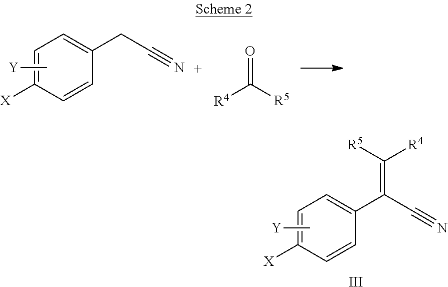

Reagents and Conditions:

If R⁵ is H, aq. NaOH, iPrOH, room temperature, 5 min or NaOMe, MeOH, 50° C., 3 h

As is illustrated in Scheme 3 below, pyrrolidines of formula IV can be made from intermediates II and III by a convergent 1,3-dipolar cycloaddition reaction mediated by lewis acid AgF and triethylamine, followed by hydrolysis. The [2+3] cycloaddition reactions of azomethine ylides 1,3-dipoles (that were generated from reacting intermediate II with AgF) with olefinic dipolarphiles for formula III to form pyrrolidine ring formation are described in the literature, including Jorgensen, K. A. et al (Org. Lett. 2005, Vol 7, No. 21, 4569-4572), Grigg, R. et al (Tetrahedron, 1992, Vol 48, No. 47, 10431-10442; Tetrahedron, 2002, Vol 58, 1719-1737), Schreiber, S. L. et al (J. Am. Chem. Soc., 2003, 125, 10174-10175), and Carretero, J. C. et al (Tetrahedron, 2007, 63, 6587-6602). Compounds of formula IV are subsequently converted to compounds of formula V by amide formation with various amines using HATU as the coupling reagent. R³ groups can be introduced to the ring nitrogen of formula V by known reactions in the literature such as reductive amination, acylation to form the compounds of formula I. The amide formation from IV to V can also be achieved under other conditions using EDCI and HOBt or oxalyl chloride as the coupling reagent to activate the acid IV.

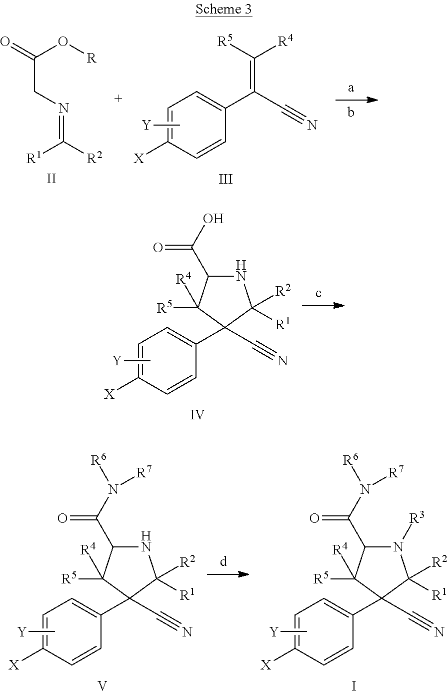

### Reagents and Conditions:

a. AgF, NEt₃, CH₂Cl₂ or ClCH₂CH₂Cl, rt, 18 h; b. 1) If R is tert-butyl, conc. H₂SO₄; or TFA, CH₂Cl₂, rt, 18 h; or 2) If R is methyl, NaOH or LiOH, H₂O and MeOH and THF, rt, 18 h; c. HNR₆R₇, HATU, iPr₂NEt, CH₂Cl₂, rt, 18 h; d. Aldehydes/NaB(AcO)₃H/AcOH/r.t.

The pyrrolidine compounds of formulas I, IV, and V are prepared initially as a racemic mixture and subsequently can be chirally separated using chiral Super Fluid Chromatography (SFC) or chiral HPLC or chiral column chromatography. For example, a racemic mixture of compounds IVa and IVa′ can be readily resolved into two optically pure or enriched chiral enantiomers by separation using chiral Super Fluid Chromatography (SFC). (see Scheme 4 below).

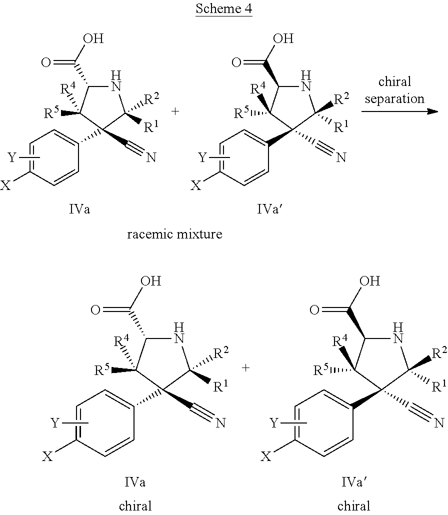

Resolution methods are well known, and are summarized in “Enantiomers, Racemates, and Resolutions” (Jacques, J. et. al. John Wiley and Sons, NY, 1981). Methods for chiral HPLC are also well known, and are summarized in “Separation of Enantiomers by Liquid Chromatographic Methods” (Rirkle, W. H. and Finn, J in Asymmetric Synthesis’” Vol. 1, Morrison, J. D., Ed. Academic Press, In., NY 1983, pp. 87-124).

Converting a Compound of Formula I that Bears a Basic Nitrogen into a Pharmaceutically Acceptable Acid Addition Salt

The optional conversion of a compound of formula I that bears a basic nitrogen into a pharmaceutically acceptable acid addition salt can be effected by conventional means. For example, the compound can be treated with an inorganic acid such as for example hydrochloric acid, hydrobromic acid, sulfuric acid, nitric acid, phosphoric acid, or with an appropriate organic acid such as acetic acid, citric acid, tartaric acid, methanesulfonic acid, p-toluene sulfonic acid, or the like.

Converting a Compound of Formula I that Bears a Carboxylic Acid Group into a Pharmaceutically Acceptable Alkali Metal Salt

The optional conversion of a compound of formula I that bears a carboxylic acid group into a pharmaceutically acceptable alkali metal salt can be effected by conventional means. For example, the compound can be treated with an inorganic base such as lithium hydroxide, sodium hydroxide, potassium hydroxide, or the like.

### Crystal Forms

When the compounds of the invention are solids, it is understood by those skilled in the art that these compounds, and their salts, may exist in different crystal or polymorphic forms, all of which are intended to be within the scope of the present invention and specified formulas.

### EXAMPLES

The compounds of the present invention may be synthesized according to known techniques. The following examples and references are provided to aid the understanding of the present invention. The examples are not intended, however, to limit the invention, the true scope of which is set forth in the appended claims. The names of the final products in the examples were generated using Isis AutoNom 2000.

Abbreviations Used in the Examples:

### HRMS: High Resolution Mass Spectrometry

### LCMS: Liquid Chromatography Mass Spectrometry

CEM microwave reactor (ex 1e): Company name
HATU: 2-(7-Azabenzotriazol-1-yl)-n,n,n′,n′-tetramethyluronium hexafluorophosphate
RT (or rt) Room temperature
PPTS: Pyridinium 4-toluenesulfonate
DBU: 1,8-Diazabicyclo[5,4,0]undec-7-ene
DIBAL: Diisobutylalumiunum hydride
iPA: Isopropyl alcohol
ASDI: ASDI-Intermediates (company name)
RP-HPLC: Reverse phase HPLC

### Min: Minutes

### H: Hours

### GST; Glutathione S-transferase

TRF: Time resolved fluorescensce

### Example 1a

### Preparation of Intermediate [3,3-dimethyl-but-(E)-ylideneamino]-acetic acid tert-butyl ester

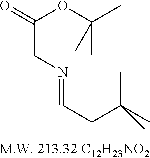

A mixture of glycine tert-butyl ester (Alfa) (2.71 g, 20.0 mmol) and 3,3-dimethyl-butyraldehyde (Alfa) (2.21 g, 21.0 mmol) in CH₂Cl₂ (50 mL) was stirred at rt overnight. The reaction mixture was concentrated and the residue was dried in vacuo to give [3,3-dimethyl-but-(E)-ylideneamino]-acetic acid tert-butyl ester (4.29 g, 100%) as colorless oil which was used in the next step without further purification.

### Example 1b

### Preparation of Intermediate (Z)-3-(3-chloro-phenyl)-2-(4-chloro-phenyl)-acrylonitrile

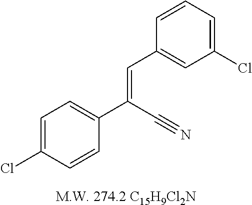

### Method A.

To a solution of 4-chlorobenzyl cyanide (Aldrich, 5.62 g, 4.00 mmol) and 3-chloro-benzaldehyde (Aldrich, 6.06 g, 4.00 mmol) in iPrOH (250 mL) was added 4 N NaOH (5 mL) dropwise at rt and the reaction mixture was stirred at rt for 10 min to give a white suspension. The solid was filtered and washed with water and iPrOH and then dried overnight in vacuum to give (Z)-3-(3-chloro-phenyl)-2-(4-chloro-phenyl)-acrylonitrile (9.33 g, 85.1%) as a white powder which was used in the next step without further purification.

### Method B.

To a solution of 4-chlorobenzyl cyanide (Aldrich, 4.5 g, 30 mmol) and 3-chloro-benzaldehyde (Aldrich, 4 g, 29 mmol) in methanol (150 mL) was slowly added a methanolic solution (Aldrich, 25 wt. %) of sodium methoxide (10 mL, 44 mmol). The reaction mixture was heated and stirred at 50° C. for 3 h. The mixture became cloudy, and was cooled to room temperature and filtered. The white precipitate was washed with water, cold methanol, and then dried in vacu to give the first batch of desired product (5.5 g). The filtrate was concentrated, diluted with water, neutralized by aqueous HCl solution to “pH” 7, then extracted with ethyl acetate. The organic layer was separated, dried over MgSO₄, and concentrated. The residue was purified by chromatography (EtOAc/hexanes=1/20, then 1/10) to give the second batch of the desired product (1.6 g). The two batches were combined to give (Z)-3-(3-chloro-phenyl)-2-(4-chloro-phenyl)-acrylonitrile as a white powder (7.1 g, 88%).

HRMS (ES⁺) m/z Calcd for C₁₅H₉Cl₂N [M+]: 273.0112. found: 273.0113.

### Example 1c

### Preparation of Intermediate rac-(2R,3R,4R,5S)-3-(3-chloro-phenyl)-4-(4-chloro-phenyl)-4-cyano-5-(2,2-dimethyl-propyl)-pyrrolidine-2-carboxylic acid tert-butyl ester

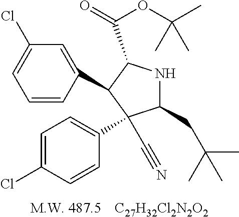

To a solution of [3,3-dimethyl-but-(E)-ylideneamino]-acetic acid tert-butyl ester (4.26 g, 20.00 mmol) and (Z)-3-(3-chloro-phenyl)-2-(4-chloro-phenyl)-acrylonitrile (5.48 g, 20.00 mmol) in ClCH₂CH₂Cl (100 mL) were added triethyl amine (4.2 g, 40.00 mmol) and AgF (Aldrich, 2.53 g, 20.00 mmol) in one portion. The mixture was stirred at rt overnight. The mixture was then quenched with sat. NH₄Cl and extracted with CH₂Cl₂. The organic phase was separated, filtered through Celite and dried over Na₂SO₄. The mixture was then separated and concentrated. The residue was triturated with EtOAc and nHexane, and the precipitates were collected by filtration and the mother liquid was concentrated and further purified by flash column (SiO₂, 1-20% of EtOAc in hexanes) to give rac-(2R,3R,4R,5S)-3-(3-chloro-phenyl)-4-(4-chloro-phenyl)-4-cyano-5-(2,2-dimethyl-propyl)-pyrrolidine-2-carboxylic acid tert-butyl ester (6.65 g, 68.2%; HRMS (ES⁺) m/z Calcd for C₂₇H₃₂Cl₂N₂O₂+H [(M+H)⁺]: 487.1914. found: 487.1910) and rac-(2R,3R,4R,5R)-3-(3-chloro-phenyl)-4-(4-chloro-phenyl)-4-cyano-5-(2,2-dimethyl-propyl)-pyrrolidine-2-carboxylic acid tert-butyl ester (0.86 g, 8.8%).

HRMS (ES⁺) m/z Calcd for C₂₇H₃₂Cl₂N₂O₂+H [(M+H)⁺]: 487.1914. found: 487.1910).

### Example 1d

### Preparation of Intermediate rac-(2R,3R,4R,5S)-3-(3-chloro-phenyl)-4-(4-chloro-phenyl)-4-cyano-5-(2,2-dimethyl-propyl)-1-ethyl-pyrrolidine-2-carboxylic acid

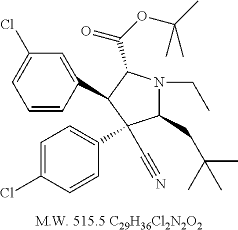

To a solution of rac-(2R,3R,4R,5S)-3-(3-chloro-phenyl)-4-(4-chloro-phenyl)-4-cyano-5-(2,2-dimethyl-propyl)-pyrrolidine-2-carboxylic acid tert-butyl ester (244.0 mg, 0.50 mmol) in AcOH (3.0 mL) was added acetaldehyde (0.5 mL) to give a yellowish solution. Sodium triacetoxyborohydride (Fluka, 1.06 g) was added in portions and the reaction mixture was stirred at rt for 4 hrs. The reaction mixture was quenched with Sat. NH₄Cl and washed with water. The residue was then purified by flash column (SiO₂, 5-20% of EtOAc in Hex) to give rac-(2R,3R,4R,5S)-3-(3-chloro-phenyl)-4-(4-chloro-phenyl)-4-cyano-5-(2,2-dimethyl-propyl)-1-ethyl-pyrrolidine-2-carboxylic acid (0.20 g, 77.5%).

### Example 1e

### Preparation of Intermediate rac-(2R,3R,4R,5S)-3-(3-chloro-phenyl)-4-(4-chloro-phenyl)-4-cyano-5-(2,2-dimethyl-propyl)-1-ethyl-pyrrolidine-2-carboxylic acid

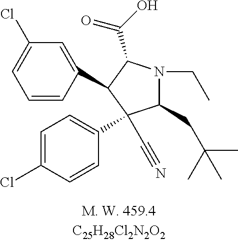

A solution of rac-(2R,3R,4R,5S)-3-(3-chloro-phenyl)-4-(4-chloro-phenyl)-4-cyano-5-(2,2-dimethyl-propyl)-1-ethyl-pyrrolidine-2-carboxylic acid tert-butyl ester (170 mg, 0.33 mmol) and conc. H₂SO₄ (2.0 mL) in MeCN (4.0 mL) was heated to 120° C. for 10 min with CEM microwave reactor. The mixture was then poured into ice and extracted with EtOAc. The organic phase was separated, dried over Na₂SO₄, and concentrated to give rac-(2R,3R,4R,5S)-3-(3-chloro-phenyl)-4-(4-chloro-phenyl)-4-cyano-5-(2,2-dimethyl-propyl)-1-ethyl-pyrrolidine-2-carboxylic acid (140.5 mg, 99.4%) as a white solid which was used in the next step without further purification: HRMS (ES⁺) m/z Calcd for C₂₅H₂₈Cl₂N₂O₂+H [(M+H)⁺]: 459.1601. found: 459.1600.

### Example 1f

### Preparation of rac-(2R,3R,4R,5S)-3-(3-chloro-phenyl)-4-(4-chloro-phenyl)-4-cyano-5-(2,2-dimethyl-propyl)-1-ethyl-pyrrolidine-2-carboxylic acid

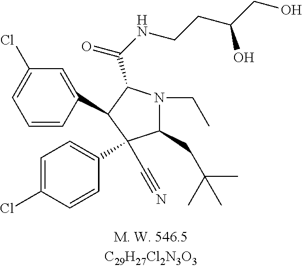

A mixture of rac-(2R,3R,4R,5S)-3-(3-chloro-phenyl)-4-(4-chloro-phenyl)-4-cyano-5-(2,2-dimethyl-propyl)-1-ethyl-pyrrolidine-2-carboxylic acid (120.0 mg, 0.26 mmol) prepared in Example 1e, 2-((S)-2,2-dimethyl-[1,3]dioxolan-4-yl)-ethylamine*(49.3 mg, 0.34 mmol), HATU (129.2 mg, 0.34 mmol) and iPr₂NEt (129.3 mg, 1.00 mmol) in CH₂Cl₂ (2 mL) was stirred at rt overnight. The mixture was then diluted with CH₂Cl₂ and washed with water, brine. The organic phase was separated, filtered and dried over Na₂SO₄. The mixture was then concentrated and the residue was treated with PPTS (cat) in MeOH (5 mL) at 120° C. for 5 min with CEM microwave reactor. The reaction mixture was concentrated and the residue was diluted with EtOAc and washed with water, brine. The organic phase was separated, filtered and dried over Na₂SO₄. The mixture was then concentrated and purified by SiO₂ flash column (5% of MeOH in EtOAc) to give rac-(2R,3R,4R,5S)-3-(3-chloro-phenyl)-4-(4-chloro-phenyl)-4-cyano-5-(2,2-dimethyl-propyl)-ethyl-pyrrolidine-2-carboxylic acid ((S)-3,4-dihydroxy-butyl)-amide (50.9 mg, 35.8%) as a white amorphous.

HRMS (ES⁺) m/z Calcd for C₂₉H₃₇Cl₂N₃O₃+H [(M+H)⁺]: 546.2285. found: 546.2283.

### *Preparation of intermediate 2-((S)-2,2-dimethyl-[1,3]dioxolan-4-yl)-ethylamine

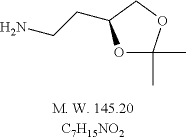

### Step A.

To a solution of (4S)-(+)-4-(2-hydroxyethyl)-2,2-dimethyl-1,3-dioxolane (Aldrich) (21.1 g, 0.14 mol) and triethylamine (40 mL, 0.28 mol) in dichloromethane (250 mL) at 0° C. was added methanesulfonyl chloride (13.4 mL, 0.17 mol) dropwise. The reaction mixture was stirred at 0° C. for 1.5 h, then water was added. The organic layer was separated, washed with water, brine, dried over MgSO₄, concentrated to give methanesulfonic acid 2-((S)-2,2-dimethyl-[1,3]dioxolan-4-yl)-ethyl ester as a yellow oil (31.7 g, 98%).

### Step B.

To a solution of methanesulfonic acid 2-((S)-2,2-dimethyl-[1,3]dioxolan-4-yl)-ethyl ester (31.7 g, 0.14 mol) in N,N-dimethylformamide (200 mL) was added NaN₃ (46 g, 0.71 mol). The reaction mixture was stirred at room temperature for 70 h. Then the mixture was partitioned between ethyl acetate and water. The organic layer was separated, washed with water, brine several times, dried over MgSO₄, concentrated to give (S)-4-(2-azido-ethyl)-2,2-dimethyl-[1,3]dioxolane as a yellow oil (21.3 g, 88%).

### Step C.

A suspension of (S)-4-(2-azido-ethyl)-2,2-dimethyl-[1,3]dioxolane as a yellow oil (18.7 g, 0.11 mol) and PtO₂ (Aldrich, 2.5 g) in ethyl acetate (100 mL) was vigorously shaken in a Parr under atmosphere of H₂ (50 psi) for 18 h. The mixture was filtered through a short pad of celite. The filtrate was concentrated to give 2-((S)-2,2-dimethyl-[1,3]dioxolan-4-yl)-ethylamine as a colorless oil (14 g, 88%).

### Example 2a

### Preparation of Intermediate eth-(E)-ylideneamino-acetic acid tert-butyl ester

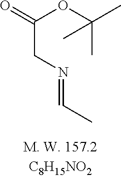

A mixture of glycine tert-butyl ester (Alfa) (4.45 g, 34.0 mmol) and acetaldehyde (Alfa) (1.91 mL, 34.0 mmol) and Sodium sulfate (24 g, 170 mmol) in CH₂Cl₂ (80 mL) was stirred at −5° C. for 1.5 hours. The reaction mixture was filtered and the filtrate was used in the next step without further purification.

### Example 2b

### Preparation of Intermediate (Z)-3-(3-chloro-2-fluoro-phenyl)-2-(4-chloro-2-fluoro-phenyl)-acrylonitrile

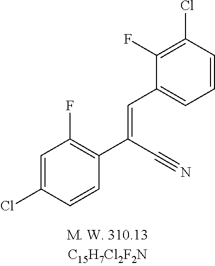

In a manner similar to the method described in Example 1b, 4-chloro-2-fluorophenylacetonitrile (5 g, 30 mmol) was reacted with 3-chloro-2-fluorobenzaldehyde (5 g, 32 mmol), methanolic solution (25 wt %) of sodium methoxide (21 mL, 92 mmol) in methanol (200 mL) at 45° C. for 5 hrs to give (Z)-3-(3-chloro-2-fluoro-phenyl)-2-(4-chloro-2-fluoro-phenyl)-acrylonitrile as a white powder (9 g, 97%).

### Example 2c

### Preparation of Intermediate rac-(2R,3S,4R,5R)-3-(3-chloro-2-fluoro-phenyl)-4-(4-chloro-2-fluoro-phenyl)-4-cyano-5-methyl-pyrrolidine-2-carboxylic acid tert-butyl ester and rac-(2R,3S,4R,5S)-3-(3-chloro-2-fluoro-phenyl)-4-(4-chloro-2-fluoro-phenyl)-4-cyano-5-methyl-pyrrolidine-2-carboxylic acid tert-butyl ester

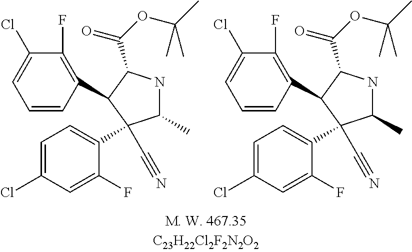

To a solution of eth-(E)-ylideneamino-acetic acid tert-butyl ester prepared in Example 2a (34 mmol) in CH₂Cl₂ (100 mL) was added (Z)-3-(3-chloro-2-fluoro-phenyl)-2-(4-chloro-2-fluoro-phenyl)-acrylonitrile prepared in Example 2b (3.43 g, 11.5 mmol), AgF (1.12 g, 8.84 mmol), and triethylamine (3.85 mL, 27.63 mmol). The reaction was stirred at room temperature for 18 h. The reaction mixture was filtered through a silica gel pad and was eluted with CH₂Cl₂ and EtOAc. The filtrate was concentrated to give an oil, which was purified by chromatography (EtOAc/hexanes 10% to 25%) to give (2R,3S,4R,5R)-3-(3-chloro-2-fluoro-phenyl)-4-(4-chloro2-fluoro-phenyl)-4-cyano-5-methyl-pyrrolidine-2-carboxylic acid tert-butyl ester as a white foam (1.79 g, 35% yield) MS (ES⁺) m/z [(M+H)⁺] 467; and rac-(2R,3S,4R,5S)-3-(3-chloro-2-fluoro-phenyl)-4-(4-chloro-2-fluoro-phenyl)-4-cyano-5-methyl-pyrrolidine-2-carboxylic acid tert-butyl ester as a white foam (2.14 g, 41% yield). MS (ES⁺) m/z [(M+H)⁺] 467.

### Example 2d

### Preparation of Intermediate rac-(2R,3S,4R,5R)-3-(3-chloro-2-fluoro-phenyl)-4-(4-chloro-2-fluorophenyl)-4-cyano-5-methyl-pyrrolidine-2-carboxylic acid; compound with trifluoro-acetic acid

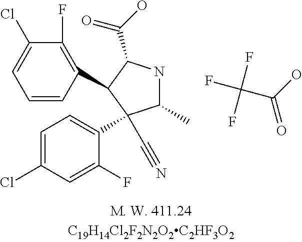

To a solution of rac-(2R,3S,4R,5R)-3-(3-chloro-2-fluoro-phenyl)-4-(4-chloro2-fluoro-phenyl)-4-cyano-5-methyl-pyrrolidine-2-carboxylic acid tert-butyl ester prepared in Example 2c (1.79 g, 3.83 mmol) in dichloromethane (30 mL) was added trifluoroacetic acid (10 mL). The reaction mixture was stirred at room temperature for 18 h, and concentrated. The residue was then triturated with ethyl ether hexanes, concentrated, dried under reduced pressure to give rac-(2R,3S,4R,5R)-3-(3-chloro-2-fluoro-phenyl)-4-(4-chloro-2-fluorophenyl)-4-cyano-5-methyl-pyrrolidine-2-carboxylic acid; compound with trifluoro-acetic acid as a white solid (1.69 g, 84%). MS (ES⁺) m/z [(M+H)⁺] 411.

### Example 2e

### Preparation of Intermediate rac-(2R,3S,4R,5S)-3-(3-chloro-2-fluoro-phenyl)-4-(4-chloro-2-fluorophenyl)-4-cyano-5-methyl-pyrrolidine-2-carboxylic acid; compound with trifluoro-acetic acid

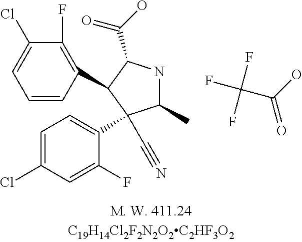

In a manner similar to the method described in Example 2d rac-(2R,3S,4R,5S)-3-(3-Chloro-2-fluoro-phenyl)-4-(4-chloro2-fluoro-phenyl)-4-cyano-5-methyl-pyrrolidine-2-carboxylic acid tert-butyl ester prepared in Example 2c (2.12 g, 4.58 mmol) in dichloromethane (30 mL) was reacted with trifluoroacetic acid (10 mL) at room temperature for 18 h to give rac-(2R,3S,4R,5S)-3-(3-Chloro-2-fluoro-phenyl)-4-(4-chloro-2-fluorophenyl)-4-cyano-5-methyl-pyrrolidine-2-carboxylic acid; compound with trifluoro-acetic acid as a white solid (2.01 g, 84%). MS (ES⁺) m/z [(M+H)⁺] 411.

### Example 2f

### Preparation of Intermediate rac-(2R,3S,4R,5S)-3-(3-chloro-2-fluoro-phenyl)-4-(4-chloro-2-fluoro-phenyl)-4-cyano-5-methyl-pyrrolidine-2-carboxylic acid [2-(2,2-dimethyl-[1,3]dioxolan-4-yl)-ethyl]-amide (p230)

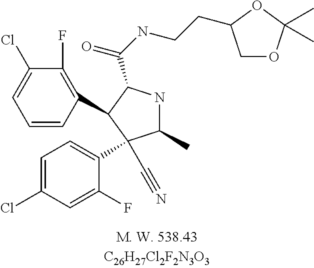

In a manner similar to the method described in Examples 1f, rac-(2R,3S,4R,5S)-3-(3-chloro-2-fluoro-phenyl)-4-(4-chloro-2-fluorophenyl)-4-cyano-5-methyl-pyrrolidine-2-carboxylic acid; compound with trifluoro-acetic acid prepared in Example 2e (1.01 g, 1.92 mmol) was reacted with 2-(2,2-dimethyl-[1,3]dioxolan-4-yl)-ethylamine prepared in Example 1f (0.44 g, 3 mmol), HATU (1.52 g, 4 mmol) and iPr₂NEt (0.7 mL, 4 mmol) in CH₂Cl₂ (40 mL) at room temperature to give rac-(2R,3S,4R,5S)-3-(3-chloro-2-fluoro-phenyl)-4-(4-chloro-2-fluoro-phenyl)-4-cyano-5-methyl-pyrrolidine-2-carboxylic acid [2-(2,2-dimethyl-[1,3]dioxolan-4-yl)-ethyl]-amide as a gum which was directly used for the next step.

### Example 2g

### Preparation of rac-(2R,3S,4R,5S)-3-(3-chloro-2-fluoro-phenyl)-4-(4-chloro-2-fluoro-phenyl)-4-cyano-5-methyl-1-propionyl-pyrrolidine-2-carboxylic acid (3,4-dihydroxy-butyl)-amide

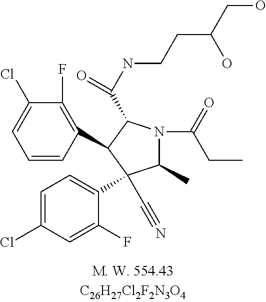

A solution of rac-(2R,3S,4R,5S)-3-(3-chloro-2-fluoro-phenyl)-4-(4-chloro-2-fluoro-phenyl)-4-cyano-5-methyl-pyrrolidine-2-carboxylic acid [2-(2,2-dimethyl-[1,3]dioxolan-4-yl)-ethyl]-amide prepared in Example 2f (104 mg, 0.21 mmol) was combined with propionyl chloride (Aldrich) (32.2 mg, 0.35 mmol) and triethyl amine (0.1 mL, 0.6 mmol). The reaction was stirred at 0° C. for 0.5 hours, washed with 0.5 N NaHCO₃ solution. The aqueous was extracted with CH₂Cl₂ (3×10 mL). The combined organic solution was dried with Na₂SO₄, filtered and concentrate to give a residue which was used directly for the next step.

To the residue was added THF (2 mL) and 6N HCl (2 mL, 0.12 mmol). The reaction was stirred at room temperature for 2 hours and was concentrated and freeze dried to give rac-(2R,3S,4R,5S)-3-(3-chloro-2-fluoro-phenyl)-4-(4-chloro-2-fluoro-phenyl)-4-cyano-5-methyl-1-propionyl-pyrrolidine-2-carboxylic acid (3,4-dihydroxy-butyl)-amide as a solid (61 mg, 85% yield). MS (ES⁺) m/z [(M+H)⁺]: 554.

### Example 3

### Preparation of rac-(2R,3S,4R,5S)-3-(3-chloro-2-fluoro-phenyl)-4-(4-chloro-2-fluoro-phenyl)-4-cyano-1-(2-ethoxy-benzoyl)-5-methyl-pyrrolidine-2-carboxylic acid (3,4-dihydroxy-butyl)-amide

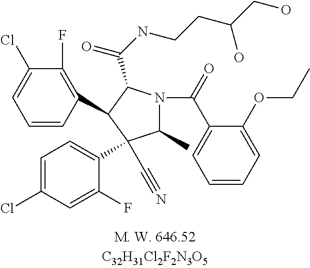

In a manner similar to the method described in Examples 2g rac-(2R,3S,4R,5S)-3-(3-chloro-2-fluoro-phenyl)-4-(4-chloro-2-fluoro-phenyl)-4-cyano-5-methyl-pyrrolidine-2-carboxylic acid [2-(2,2-dimethyl-[1,3]dioxolan-4-yl)-ethyl]-amide prepared in Example 2f (0.10 g, 0.19 mmol) was reacted with 2-ethoxybenzoyl chloride (Aldrich, excess) and triethyl amine (0.1 mL, 0.6 mmol) in CH₂Cl₂ (3 mL) to give a residue which was then reacted with 6N HCl (1.6 mL, 9.6 mmol) in THF (5 mL) to give rac-(2R,3S,4R,5S)-3-(3-chloro-2-fluoro-phenyl)-4-(4-chloro-2-fluoro-phenyl)-4-cyano-1-(2-ethoxy-benzoyl)-5-methyl-pyrrolidine-2-carboxylic acid (3,4-dihydroxy-butyl)-amide as a white solid (61 mg, 51% yield). MS (ES⁺) m/z [(M+H)⁺]: 646

### Example 4

### Preparation of rac-(2R,3S,4R,5S)-3-(3-chloro-2-fluoro-phenyl)-4-(4-chloro-2-fluoro-phenyl)-4-cyano-1-(3-ethoxy-benzoyl)-5-methyl-pyrrolidine-2-carboxylic acid (3,4-dihydroxy-butyl)-amide

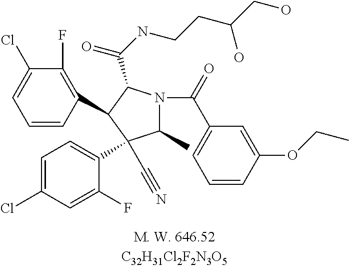

In a manner similar to the method described in Examples 2g rac-(2R,3S,4R,5S)-3-(3-chloro-2-fluoro-phenyl)-4-(4-chloro-2-fluoro-phenyl)-4-cyano-5-methyl-pyrrolidine-2-carboxylic acid [2-(2,2-dimethyl-[1,3]dioxolan-4-yl)-ethyl]-amide prepared in Example 2f (0.10 g, 0.19 mmol) was reacted with 3-ethoxybenzoyl chloride (Aldrich, 0.19 mmol) and triethyl amine (0.1 mL, 0.6 mmol) in CH₂Cl₂ (3 mL) to give a residue which was then reacted with 6N HCl (1.6 mL, 9.6 mmol) in THF (5 mL) to give rac-(2R,3S,4R,5S)-3-(3-chloro-2-fluoro-phenyl)-4-(4-chloro-2-fluoro-phenyl)-4-cyano-1-(3-ethoxy-benzoyl)-5-methyl-pyrrolidine-2-carboxylic acid (3,4-dihydroxy-butyl)-amide as an off white solid (52 mg, 43% yield). MS (ES⁺) m/z [(M+H)⁺]: 646

### Example 5

### Preparation of rac-(2R,3S,4R,5S)-3-(3-chloro-2-fluoro-phenyl)-4-(4-chloro-2-fluoro-phenyl)-4-cyano-1-(3-methyl-benzoyl)-5-methyl-pyrrolidine-2-carboxylic acid (3,4-dihydroxy-butyl)-amide

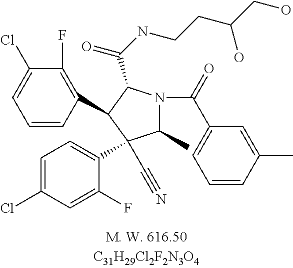

In a manner similar to the method described in Example 2g, rac-(2R,3S,4R,5S)-3-(3-chloro-2-fluoro-phenyl)-4-(4-chloro-2-fluoro-phenyl)-4-cyano-5-methyl-pyrrolidine-2-carboxylic acid [2-(2,2-dimethyl-[1,3]dioxolan-4-yl)-ethyl]-amide prepared in Example 2f (0.10 g, 0.19 mmol) was reacted with 3-methylbenzoyl chloride (Aldrich, 0.19 mmol) and triethyl amine (0.1 mL, 0.6 mmol) in CH₂Cl₂ (3 mL) to give a residue which was then reacted with 6N HCl (1.6 mL, 9.6 mmol) in THF (5 mL) to give rac-(2R,3S,4R,5S)-3-(3-chloro-2-fluoro-phenyl)-4-(4-chloro-2-fluoro-phenyl)-4-cyano-1-(3-methyl-benzoyl)-5-methyl-pyrrolidine-2-carboxylic acid (3,4-dihydroxy-butyl)-amide as a brown solid (56 mg, 49% yield). MS (ES⁺) m/z [(M+H)⁺]: 616

### Example 6a

### Preparation of Intermediate 4-[(E)-tert-butoxycarbonylmethylimino]-2,2-dimethyl-butyric acid methyl ester

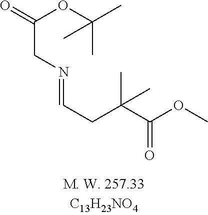

### Step A

A mixture of 2,2-dimethylbutyrolactone (6.84 g, 60 mmol) and KOH (3.36 g) in H₂O (60 mL) was heated at reflux for 2 h. The solution was cooled to room temperature, and acidified to “pH” 5 with aqueous HCl solution. The mixture was then extracted with ethyl acetate three times. The combined organic layers were washed with brine, dried over MgSO₄, concentrated under reduced pressure to give 4-hydroxy-2,2-dimethyl-butanoic acid as a colorless oil (4 g, 51%).

### Step B

To the mixture of 4-hydroxy-2,2-dimethyl-butanoic acid (2.2 g, 16.6 mmol) in ethyl ether (16 mL) and methanol (24 mL) at 0° C. was added a hexane solution (2.0 M) of trimethylsilyldiazomethane (Aldrich) (12.5 mL, 25 mmol). The reaction mixture was stirred at 0° C. for 1 h. The solvents were evaporated. The residue was taken up in ethyl acetate, washed with diluted aqueous HCl solution, saturated aqueous NaHCO₃ solution, brine, dried over MgSO₄, and concentrated to give 4-hydroxy-2,2-dimethyl-butyric acid methyl ester as a colorless oil (1.5 g, 62%).

### Step C

To a solution of oxalyl chloride (1.09 mL, 12.5 mmol) (Aldrich) in dichloromethane (20 mL) at −78° C. was added the solution of dimethyl sulfoxide (1.77 mL, 25 mmol) in dichloromethane (5 mL) dropwise. After 5 mins, the solution of 4-hydroxy-2,2-dimethyl-butyric acid methyl ester (1.5 g, 11.3 mmol) in dichloromethane (5 mL) was added dropwise. The reaction mixture was stirred at −78° C. for 15 min. Triethylamine (5.7 mL, 41 mmol) was added and the reaction mixture was slowly warmed up to room temperature and stirred at room temperature for 45 min. Then water was added. The organic layers were separated, and the aqueous layer was extracted with dichloromethane. The organic layers were combined, washed with 10% of HCl, saturated NaHCO₃, brine, dried over MgSO₄, and concentrated to give 2,2-dimethyl-4-oxo-butyric acid methyl ester as a light yellow oil (Yield: 1.2 g, 81%).

### Step D

In a manner similar to the method described in Example 1a, glycine tert-butyl ester (1.09 g, 8.32 mmol) was reacted with 2,2-dimethyl-4-oxo-butyric acid methyl ester (1.2 g, 8.32 mmol) in CH₂Cl₂ at room temperature for 18 h to give 4-[(E)-tert-butoxycarbonylmethylimino]-2,2-dimethyl-butyric acid methyl ester as a colorless oil (2.1 g, 100%).

### Example 6b

### Preparation of Intermediate rac-(2R,3S,4R,5S)-3-(3-chloro-2-fluoro-phenyl)-4-(4-chloro-2-fluoro-phenyl)-4-cyano-5-(2-methoxycarbonyl-2-methyl-propyl)-pyrrolidine-2-carboxylic acid tert-butyl ester

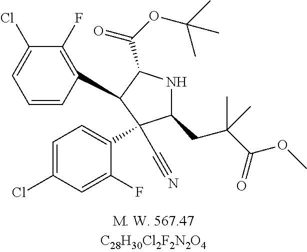

In a manner similar to the method described in Example 1c, 4-[(E)-tert-butoxy-carbonylmethylimino]-2,2-dimethyl-butyric acid methyl ester (2.1 g, 8.3 mmol) was reacted with (Z)-3-(3-chloro-2-fluoro-phenyl)-2-(4-chloro-2-fluoro-phenyl)-acrylonitrile (2.05 g, 6.7 mmol) prepared in Example 2b, AgF (Aldrich, 1.27 g, 10 mmol), and triethylamine (2.3 mL, 17 mmol) in dichloromethane (150 mL) at room temperature for 18 h, followed by the reaction with DBU (2 ml) in tert-butanol (10 mL) at 100° C. for 2 h to give rac-(2R,3S,4R,5S)-3-(3-chloro-2-fluoro-phenyl)-4-(4-chloro-2-fluoro-phenyl)-4-cyano-5-(2-methoxycarbonyl-2-methyl-propyl)-pyrrolidine-2-carboxylic acid tert-butyl ester as a white solid (0.75 g, 20%).

### Example 6c

### Preparation of Intermediate rac-(2R,3S,4R,5S)-3-(3-chloro-2-fluoro-phenyl)-4-(4-chloro-2-fluoro-phenyl)-4-cyano-5-(2-methoxycarbonyl-2-methyl-propyl)-pyrrolidine-2-carboxylic acid trifluoroacetic acid

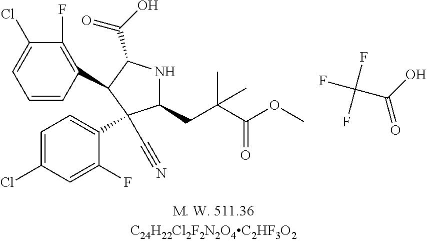

In a manner similar to the method described in Example 2d, rac-(2R,3S,4R,5S)-3-(3-chloro-2-fluoro-phenyl)-4-(4-chloro-2-fluoro-phenyl)-4-cyano-5-(2-methoxycarbonyl-2-methyl-propyl)-pyrrolidine-2-carboxylic acid tert-butyl ester (0.7 g, 1.23 mmol) was reacted with trifluoroacetic acid in dichloromethane at room temperature to give rac-(2R,3S,4R,5S)-3-(3-chloro-2-fluoro-phenyl)-4-(4-chloro-2-fluoro-phenyl)-4-cyano-5-(2-methoxycarbonyl-2-methyl-propyl)-pyrrolidine-2-carboxylic acid trifluoroacetic acid as a white solid (0.75 g, 97%).

### Example 6d

### Preparation of Intermediate rac-3-{(2S,3R,4S,5R)-4-(3-chloro-2-fluoro-phenyl)-3-(4-chloro-2-fluoro-phenyl)-3-cyano-5-[2-((S)-2,2-dimethyl-[1,3]dioxolan-4-yl)-ethylcarbamoyl]-pyrrolidin-2-yl}-2,2-dimethyl-propionic acid methyl ester

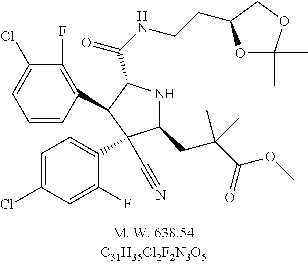

A mixture of rac-(2R,3S,4R,5S)-3-(3-chloro-2-fluoro-phenyl)-4-(4-chloro-2-fluoro-phenyl)-4-cyano-5-(2-methyl-2-phenoxycarbonyl-propyl)-pyrrolidine-2-carboxylic acid trifluoroacetic acid (0.75 g, 1.26 mmol), 2-((S)-2,2-dimethyl-[1,3]dioxolan-4-yl)-ethylamine (0.52 g, 3.6 mmol), HATU (0.82 g, 2.16 mmol) and iPr₂NEt (1.04 mL, 6 mmol) in CH₂Cl₂ (5 mL) was stirred at room temperature for 20 h. The mixture was then diluted with CH₂Cl₂ and washed with water, brine. The organic phase was separated, filtered and dried over Na₂SO₄. The mixture was then concentrated and the residue was purified by chromatography (50% EtOAc in hexanes) to give rac-3-{(2S,3R,4S,5R)-4-(3-chloro-2-fluoro-phenyl)-3-(4-chloro-2-fluoro-phenyl)-3-cyano-5-[2-((S)-2,2-dimethyl-[1,3]dioxolan-4-yl)-ethylcarbamoyl]-pyrrolidin-2-yl}-2,2-dimethyl-propionic acid methyl ester as a white solid (0.45 g, 59%).

### Example 6e

### Preparation of Intermediate rac-(1R,2S,3R,7aS)-1-(4-chloro-2-fluoro-phenyl)-2-(3-chloro-2-fluoro-phenyl)-1-cyano-6,6-dimethyl-5-oxo-hexahydro-pyrrolizine-3-carboxylic acid [2-((S)-2,2-dimethyl-[1,3]dioxolan-4-yl)-ethyl]-amide

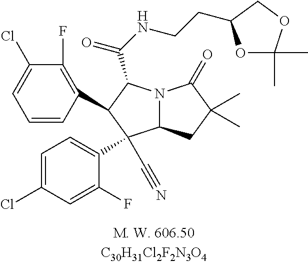

To a solution of rac-3-{(2S,3R,4S,5R)-4-(3-chloro-2-fluoro-phenyl)-3-(4-chloro-2-fluoro-phenyl)-3-cyano-5-[2-((S)-2,2-dimethyl-[1,3]dioxolan-4-yl)-ethylcarbamoyl]-pyrrolidin-2-yl}-2,2-dimethyl-propionic acid methyl ester (0.4 g, 0.63 mmol) in tetrahydrofuran (5 mL) was added an aqueous solution (5 mL) of LiOH.H₂O (0.26 g, 6.3 mmol). The reaction mixture was heated at 80° C. for 3 h. The solvent was removed, and the residue was partitioned between ethyl acetate and water. The organic layer was separated, and aqueous layer was extracted with ethyl acetate several times. The extracts were combined, washed with water, brine, dried over MgSO₄, and concentrated to give rac-(1R,2S,3R,7aS)-1-(4-chloro-2-fluoro-phenyl)-2-(3-chloro-2-fluoro-phenyl)-1-cyano-6,6-dimethyl-5-oxo-hexahydro-pyrrolizine-3-carboxylic acid [2-((S)-2,2-dimethyl-[1,3]dioxolan-4-yl)-ethyl]-amide as a colorless oil (0.35 g, 92%).

### Example 6f

### Preparation of rac-(1R,2S,3R,7aS)-1-(4-chloro-2-fluoro-phenyl)-2-(3-chloro-2-fluoro-phenyl)-1-cyano-6,6-dimethyl-5-oxo-hexahydro-pyrrolizine-3-carboxylic acid ((S)-3,4-dihydroxy-butyl)-amide

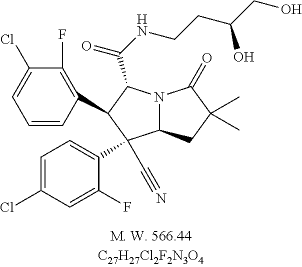

To a solution of rac-(1R,2S,3R,7aS)-1-(4-chloro-2-fluoro-phenyl)-2-(3-chloro-2-fluoro-phenyl)-1-cyano-6,6-dimethyl-5-oxo-hexahydro-pyrrolizine-3-carboxylic acid [2-((S)-2,2-dimethyl-[1,3]dioxolan-4-yl)-ethyl]-amide (0.35 g, 0.58 mol) in tetrahydrofuran (9 mL) was added aqueous HCl solution (1N, 1 mL). The reaction mixture was stirred at room temperature for 2 h, then concentrated. Then the residue was partitioned between ethyl acetate and water. The organic layer was separated, washed with water, aqueous saturated NaHCO₃, brine, dried over MgSO₄, concentrated, dried under reduced pressure to give rac-(1R,2S,3R,7aS)-1-(4-chloro-2-fluoro-phenyl)-2-(3-chloro-2-fluoro-phenyl)-1-cyano-6,6-dimethyl-5-oxo-hexahydro-pyrrolizine-3-carboxylic acid ((S)-3,4-dihydroxy-butyl)-amide as a white solid (0.29 g, 88%). HRMS (ES⁺) m/z Calcd for C₂₇H₂₇Cl₂F₂N₃O₄+H [(M+H)⁺]: 566.1420. found: 566.1419.

### Example 7a

### Preparation of Intermediate [3,3-dimethyl-but-(E)-ylideneamino]-acetic acid methyl ester

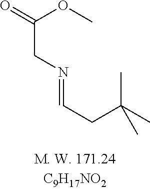

To a suspension of glycine methyl ester hydrochloride (Fluka) (1.6 g, 12.8 mmol) in dichloromethane (50 mL) was added triethylamine (1.3 g, 12.8 mmol). The mixture was stirred at room temperature for 1 h, and 3,3-dimethyl-butyraldehyde (Aldrich) (1.3 g, 12.8 mmol) was added. The reaction mixture was stirred at room temperature for 4 h. The mixture was washed with water, and the organic layer was separated, dried over MgSO₄, and concentrated to give [3,3-dimethyl-but-(E)-ylideneamino]-acetic acid methyl ester (2.2 g, 100%) as colorless oil which was used in the next step without further purification.

### Example 7b

### Preparation of Intermediate rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid methyl ester

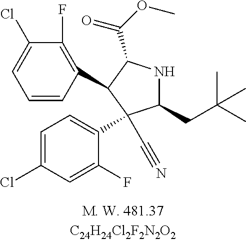

In a manner similar to the method described in Example 1c, [3-methyl-but-(E)-ylideneamino]-acetic acid methyl ester (2.2 g, 12.8 mmol) was reacted with (Z)-3-(3-chloro-2-fluoro-phenyl)-2-(4-chloro-2-fluoro-phenyl)-acrylonitrile (3.1 g, 10.0 mmol) prepared in Example 2b, AgF (1.4 g, 11.0 mmol), and triethylamine (10 g, 99 mmol) in dichloromethane (100 mL) at room temperature for 18 h to give rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid methyl ester as a white foam (3.4 g, 71%).

### Example 7c

### Preparation of Intermediate rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid methyl ester

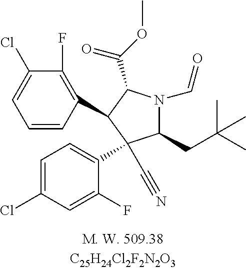

A mixture of rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid methyl ester (3.4 g, 7.1 mmol) in formic acid (Acros, 99%, 100 g) was heated at 90° C. for 24 h. The mixture was cooled to room temperature and concentrated. The residue was dissolved in ethyl acetate, washed with water, aqueous saturated NaHCO₃, and brine. The organic phase was separated, dried over Na₂SO₄, and concentrated. The residue was purified by chromatography (25% EtOAc in hexanes) to give rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid methyl ester as a white form (2.3 g, 64%).

### Example 7d

### Preparation of Intermediate rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid

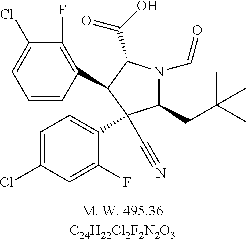

To a solution of rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid methyl ester (1 g, 2 mmol) in tetrahydrofuran (45 mL) was added an aqueous solution (15 mL) of NaOH (0.4 g, 10 mmol). The reaction mixture was stirred at room temperature for 3 h. The “pH” of the mixture was adjusted to 5-6 by diluted aqueous HCl solution. The mixture was concentrated, and the residue was partitioned between ethyl acetate and water. The organic layer was separated, and aqueous layer was extracted with ethyl acetate several times. The extracts were combined, washed with water, brine, dried over MgSO₄, and concentrated to give rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid as a white foam (0.9 g, 90%).

### Example 7e

### Preparation of Intermediate rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid [2-((S)-2,2-dimethyl-[1,3]dioxolan-4-yl)-ethyl]-amide

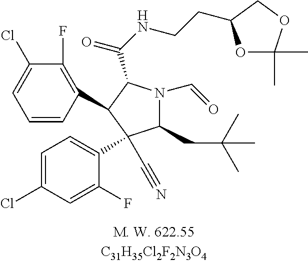

A mixture of rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid (0.33 g, 0.67 mmol), 2-((S)-2,2-dimethyl-[1,3]dioxolan-4-yl)-ethylamine (0.29 g, 2 mmol), HATU (0.46 g, 1.2 mmol) and iPr₂NEt (0.35 mL, 2 mmol) in CH₂Cl₂ (10 mL) was stirred at room temperature for 20 h. The mixture was then diluted with CH₂Cl₂ and washed with water, brine. The organic phase was separated, filtered and dried over Na₂SO₄. The mixture was then concentrated and the residue was purified by chromatography (50% EtOAc in hexanes) to give rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid [2-((S)-2,2-dimethyl-[1,3]dioxolan-4-yl)-ethyl]-amide as a white gum (0.28 g, 67%).

### Example 7f

### Preparation of rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid ((S)-3,4-dihydroxy-butyl)-amide

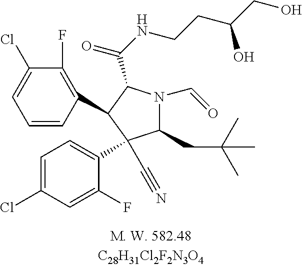

To a solution of rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid [2-((S)-2,2-dimethyl-[1,3]dioxolan-4-yl)-ethyl]-amide (0.28 g, 0.45 mol) in tetrahydrofuran (7 mL) was added aqueous HCl solution (1 N, 3 mL). The reaction mixture was stirred at room temperature for 2 h, then concentrated. Then the residue was partitioned between ethyl acetate and water. The organic layer was separated, washed with water, aqueous saturated NaHCO₃, brine, dried over MgSO₄, concentrated, dried under reduced pressure to give rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid ((S)-3,4-dihydroxy-butyl)-amide as a white solid (0.21 g, 81%). HRMS (ES⁺) m/z Calcd for C₂₈H₃₁Cl₂F₂N₃O₄+H [(M+H)⁺]: 582.1733. found: 582.1732.

### Example 8a

### Preparation of Intermediate [4-(tert-butyl-dimethyl-silanyloxy)-3,3-dimethyl-but-(E)-ylideneamino]-acetic acid tert-butyl ester

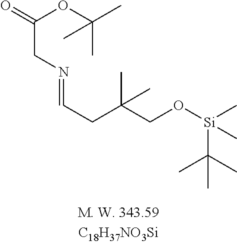

### Step A

A mixture of 2,2-dimethyl-propane-1,3-diol (Aldrich) (10 g, 96 mmol) and imidazole (9.8 g, 140 mmol) in dichloromethane (200 mL) was added tert-butyldimethylchlorosilane (15.9 g, 10.6 mmol). The reaction mixture was stirred at room temperature for 0.5 h. Water was added. The organic layer was separated, the aqueous layer was then extracted with dichloromethane. The combined organic layers were washed with brine, dried over MgSO₄, concentrated to give 3-(tert-butyl-dimethyl-silanyloxy)-2,2-dimethyl-propan-1-ol as a colorless oil (20.4 g, 97%).

### Step B

To the solution of 3-(tert-butyl-dimethyl-silanyloxy)-2,2-dimethyl-propan-1-ol (20.4 g, 93 mmol) and triethylamine (26 g, 186 mmol) in dichloromethane (200 mL) at 0° C. was added a dichlormethane solution (20 mL) of methanesulfonyl chloride (Aldrich) (8.69 mL, 112 mmol). The reaction mixture was stirred at 0° C. for 2 h. Water was added. Organic layer was separated, the aqueous layer was extracted with dichlormethane. The combined organic layers were washed with diluted aqueous HCl solution, saturated aqueous NaHCO₃ solution, brine, dried over MgSO₄, and concentrated to give methanesulfonic acid 3-(tert-butyl-dimethyl-silanyloxy)-2,2-dimethyl-propyl ester as a yellow oil (24 g, 87%).

### Step C

To the solution of methanesulfonic acid 3-(tert-butyl-dimethyl-silanyloxy)-2,2-dimethyl-propyl ester (5 g, 16.8 mmol) in anhydrous dimethyl sulfoxide (50 mL) was added KCN (2.85 g, 44 mmol). The reaction mixture was heated at 120° C. for 16 h. The mixture was cooled, and water was added. The mixture was extracted with ethyl acetate twice. The combined organic layers were washed with saturated aqueous NaHCO₃ solution, brine, dried over MgSO₄, and concentrated. The residue was purified by chromatography (EtOAc:hexanes=1; 4) to give 4-(tert-butyl-dimethyl-silanyloxy)-3,3-dimethyl-butyronitrile as a yellow oil (2.2 g, 57%).

### Step D

To a solution of 4-(tert-butyl-dimethyl-silanyloxy)-3,3-dimethyl-butyronitrile (2.2 g, 9.67 mmol) (Aldrich) in dichloromethane (20 mL) at −78° C. was added a toluene solution (1 M) of DIBAL (10.6 mL, 10.6 mmol) dropwise. The reaction mixture was stirred at 0° C. for 3 h. The mixture was poured into aqueous saturated NH₄Cl solution. The organic layer was separated, and the aqueous layer was extracted with ethyl acetate. The organic layers were combined, washed with brine, dried over MgSO₄, and concentrated. The residue was purified by chromatography (EtOAc:hexanes=1:4) to give 4-(tert-butyl-dimethyl-silanyloxy)-3,3-dimethyl-butyraldehyde as a colorless oil (Yield: 0.84 g, 38%).

### Step E

In a manner similar to the method described in Example 1a, glycine tert-butyl ester (0.52 g, 3.64 mmol) was reacted with 4-(tert-butyl-dimethyl-silanyloxy)-3,3-dimethyl-butyraldehyde (0.84 g, 3.64 mmol) in CH₂Cl₂ at room temperature for 18 h to give [4-(tert-butyl-dimethyl-silanyloxy)-3,3-dimethyl-but-(E)-ylideneamino]-acetic acid tert-butyl ester as a colorless oil (1.25 g, 100%).

### Example 8b

### Preparation of Intermediate rac-(2R,3S,4R,5S)-5-[3-(tert-butyl-dimethyl-silanyloxy)-2,2-dimethyl-propyl]-3-(3-chloro-2-fluoro-phenyl)-4-(4-chloro-2-fluoro-phenyl)-4-cyano-pyrrolidine-2-carboxylic acid tert-butyl ester

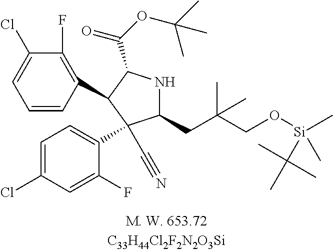

To a solution of [4-(tert-butyl-dimethyl-silanyloxy)-3,3-dimethyl-but-(E)-ylideneamino]-acetic acid tert-butyl ester (1.25 g, 3.64 mmol) and (Z)-3-(3-chloro-2-fluoro-phenyl)-2-(4-chloro-2-fluoro-phenyl)-acrylonitrile (0.93 g, 3 mmol) prepared in Example 2b in dichloromethane (100 mL) were added AgF (0.57 g, 4.5 mmol), and triethylamine (1.05 mL, 7.5 mmol). The mixture was stirred at room temperature for 18 h. The mixture was then quenched with sat. NH₄Cl and extracted with CH₂Cl₂. The organic phase was separated, filtered through celite and dried over Na₂SO₄, and concentrated. The residue was dissolved into tert-butanol (15 mL), and DBU (3.6 mL) was added. The mixture was heated at 100° C. for 2 h, then cooled to room temperature, and concentrated. The residue was partitioned between ethyl acetate and water. The organic layer were separated, dried over MgSO₄, and concentrated. The residue was purified by chromatography (EtOAc:hexanes=1; 10, 1:3) to give rac-(2R,3S,4R,5S)-5-[3-(tert-butyl-dimethyl-silanyloxy)-2,2-dimethyl-propyl]-3-(3-chloro-2-fluoro-phenyl)-4-(4-chloro-2-fluoro-phenyl)-4-cyano-pyrrolidine-2-carboxylic acid tert-butyl ester as a white solid (1.2 g, 61%).

### Example 8c

### Preparation of Intermediate rac-(2R,3S,4R,5S)-3-(3-chloro-2-fluoro-phenyl)-4-(4-chloro-2-fluoro-phenyl)-4-cyano-5-(3-hydroxy-2,2-dimethyl-propyl)-pyrrolidine-2-carboxylic acid trifluoroacetic acid

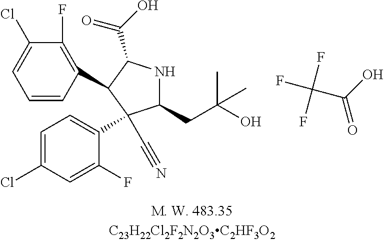

To a solution of rac-(2R,3S,4R,5S)-5-[3-(tert-butyl-dimethyl-silanyloxy)-2,2-dimethyl-propyl]-3-(3-chloro-2-fluoro-phenyl)-4-(4-chloro-2-fluoro-phenyl)-4-cyano-pyrrolidine-2-carboxylic acid tert-butyl ester (1.1 g, 1.68 mmol) in dichloromethane (30 mL) was added trifluoroacetic acid (10 mL). The reaction mixture was stirred at room temperature for 18 h, and concentrated. The residue was then triturated with ethyl ether hexanes, concentrated, dried under reduced pressure to give rac-(2R,3S,4R,5S)-3-(3-chloro-2-fluoro-phenyl)-4-(4-chloro-2-fluoro-phenyl)-4-cyano-5-(3-hydroxy-2,2-dimethyl-propyl)-pyrrolidine-2-carboxylic acid trifluoroacetic acid as a white solid (1 g, 100%).

### Example 8d

### Preparation of Intermediate rac-(2R,3S,4R,5S)-3-(3-chloro-2-fluoro-phenyl)-4-(4-chloro-2-fluoro-phenyl)-4-cyano-5-(3-hydroxy-2,2-dimethyl-propyl)-pyrrolidine-2-carboxylic acid [2-((S)-2,2-dimethyl-[1,3]dioxolan-4-yl)-ethyl]-amide

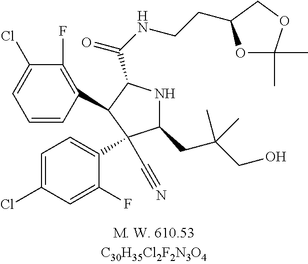

A mixture of rac-(2R,3S,4R,5S)-3-(3-chloro-2-fluoro-phenyl)-4-(4-chloro-2-fluoro-phenyl)-4-cyano-5-(3-hydroxy-2,2-dimethyl-propyl)-pyrrolidine-2-carboxylic acid trifluoroacetic acid (1 g, 1.67 mmol), 2-((S)-2,2-dimethyl-[1,3]dioxolan-4-yl)-ethylamine (0.73 g, mmol), HATU (1.14 g, 3 mmol), and iPr₂NEt (1.46 mL, 8.4 mmol) in CH₂Cl₂ (15 mL) was stirred at room temperature for 20 h. The mixture was then diluted with CH₂Cl₂ and washed with water, brine. The organic phase was separated, filtered and dried over Na₂SO₄. The mixture was then concentrated and the residue was purified by chromatography to give rac-(2R,3S,4R,5S)-3-(3-chloro-2-fluoro-phenyl)-4-(4-chloro-2-fluoro-phenyl)-4-cyano-5-(3-hydroxy-2,2-dimethyl-propyl)-pyrrolidine-2-carboxylic acid [2-((S)-2,2-dimethyl-[1,3]dioxolan-4-yl)-ethyl]-amide as a white gum (0.72 g, 71%).

### Example 8e

### Preparation of Intermediate methanesulfonic acid rac-3-{(2S,3R,4S,5R)-4-(3-chloro-2-fluoro-phenyl)-3-(4-chloro-2-fluoro-phenyl)-3-cyano-5-[2-((S)-2,2-dimethyl-[1,3]dioxolan-4-yl)-ethylcarbamoyl]-pyrrolidin-2-yl}-2,2-dimethyl-propyl ester

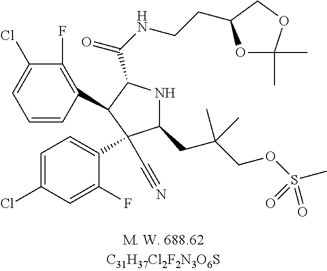

To the solution of rac-(2R,3S,4R,5S)-3-(3-chloro-2-fluoro-phenyl)-4-(4-chloro-2-fluoro-phenyl)-4-cyano-5-(3-hydroxy-2,2-dimethyl-propyl)-pyrrolidine-2-carboxylic acid [2-((S)-2,2-dimethyl-[1,3]dioxolan-4-yl)-ethyl]-amide (0.25 g, 0.41 mmol) and triethylamine (0.11 mL, 0.82 mmol) in dichloromethane (5 mL) at 0° C. was added methanesulfonyl chloride (Aldrich) (0.038 mL, 0.49 mmol). The reaction mixture was stirred at 0° C. for 0.5 h. Water was added. Organic layer was separated, the aqueous layer was extracted with dichlormethane. The combined organic layers were washed with diluted aqueous HCl solution, saturated aqueous NaHCO₃ solution, brine, dried over MgSO₄, and concentrated to give methanesulfonic acid rac-3-{(2S,3R,4S,5R)-4-(3-chloro-2-fluoro-phenyl)-3-(4-chloro-2-fluoro-phenyl)-3-cyano-5-[2-((S)-2,2-dimethyl-[1,3]dioxolan-4-yl)-ethylcarbamoyl]-pyrrolidin-2-yl}-2,2-dimethyl-propyl ester as a colorless oil (0.2 g, 71%).

### Example 8f

### Preparation of Intermediate rac-(1R,2S,3R,7aS)-1-(4-chloro-2-fluoro-phenyl)-2-(3-chloro-2-fluoro-phenyl)-1-cyano-6,6-dimethyl-hexahydro-pyrrolizine-3-carboxylic acid [2-((S)-2,2-dimethyl-[1,3]dioxolan-4-yl)-ethyl]-amide

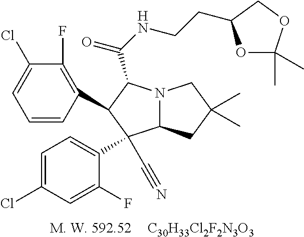

To the solution of methanesulfonic acid rac-3-{(2S,3R,4S,5R)-4-(3-chloro-2-fluoro-phenyl)-3-(4-chloro-2-fluoro-phenyl)-3-cyano-5-[2-((S)-2,2-dimethyl-[1,3]dioxolan-4-yl)-ethylcarbamoyl]-pyrrolidin-2-yl}-2,2-dimethyl-propyl ester (0.2 g, 0.29 mmol) in N,N-dimethylformamide (3 mL) was added Cs₂CO₃ (Aldrich) (0.24 g, 0.73 mmol). The reaction mixture was stirred at room temperature for 18 h. Water was added. The mixture was extracted with ethyl acetate twice. The combined organic layers were washed with water, brine, dried over MgSO₄, and concentrated. The residue was purified by chromatography to give rac-(1R,2S,3R,7aS)-1-(4-chloro-2-fluoro-phenyl)-2-(3-chloro-2-fluoro-phenyl)-1-cyano-6,6-dimethyl-hexahydro-pyrrolizine-3-carboxylic acid [2-((S)-2,2-dimethyl-[1,3]dioxolan-4-yl)-ethyl]-amide as a white solid (0.12 g, 71%).

### Example 8g

### Preparation of rac-(1R,2S,3R,7aS)-1-(4-chloro-2-fluoro-phenyl)-2-(3-chloro-2-fluoro-phenyl)-1-cyano-6,6-dimethyl-hexahydro-pyrrolizine-3-carboxylic acid ((S)-3,4-dihydroxy-butyl)-amide

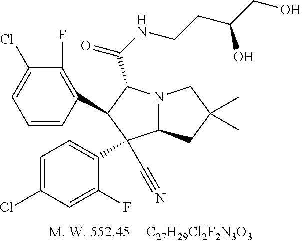

To a solution of rac-(1R,2S,3R,7aS)-1-(4-chloro-2-fluoro-phenyl)-2-(3-chloro-2-fluoro-phenyl)-1-cyano-6,6-dimethyl-hexahydro-pyrrolizine-3-carboxylic acid [2-((S)-2,2-dimethyl-[1,3]dioxolan-4-yl)-ethyl]-amide (0.12 g, 0.2 mol) in tetrahydrofuran (7 mL) was added aqueous HCl solution (1 N, 3 mL). The reaction mixture was stirred at room temperature for 2 h, then concentrated. Then the residue was partitioned between ethyl acetate and water. The organic layer was separated, washed with water, aqueous saturated NaHCO₃, brine, dried over MgSO₄, concentrated, dried under reduced pressure to give rac-(1R,2S,3R,7aS)-1-(4-chloro-2-fluoro-phenyl)-2-(3-chloro-2-fluoro-phenyl)-1-cyano-6,6-dimethyl-hexahydro-pyrrolizine-3-carboxylic acid ((S)-3,4-dihydroxy-butyl)-amide as a white solid (80 mg, 72%).

HRMS (ES⁺) m/z Calcd for C₂₇H₂₉Cl₂F₂N₃O₃+H [(M+H)⁺]: 552.1627. found: 552.1624.

### Example 9

### Preparation of rac-(2R,3S,4R,5S)-3-(3-chloro-2-fluoro-phenyl)-4-(4-chloro-2-fluoro-phenyl)-4-cyano-1-(2-methyl-benzoyl)-5-methyl-pyrrolidine-2-carboxylic acid (3,4-dihydroxy-butyl)-amide

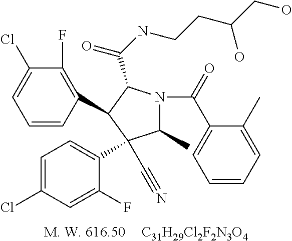

In a manner similar to the method described in Example 2g rac-(2R,3S,4R,5S)-3-(3-chloro-2-fluoro-phenyl)-4-(4-chloro-2-fluoro-phenyl)-4-cyano-5-methyl-pyrrolidine-2-carboxylic acid [2-(2,2-dimethyl-[1,3]dioxolan-4-yl)-ethyl]-amide in Example 2f (0.13 g, 0.24 mmol) was reacted with 2-methyl benzoyl chloride (Aldrich, 39 mg, 0.25 mmol) and triethyl amine (0.044 mL, 0.25 mmol) in CH₂Cl₂ (4 mL) to give a residue (42 mg) which was then reacted with 6N HCl (1.0 mL, 6 mmol) in THF (3 mL) to give rac-(2R,3S,4R,5S)-3-(3-chloro-2-fluoro-phenyl)-4-(4-chloro-2-fluoro-phenyl)-4-cyano-1-(2-methyl-benzoyl)-5-methyl-pyrrolidine-2-carboxylic acid (3,4-dihydroxy-butyl)-amide as a white solid (38 mg,). MS (ES⁺) m/z [(M+H)⁺]: 616.

### Example 10a

### Preparation of Intermediate rac-4-{[(2R,3S,4R,5R)-3-(3-chloro-2-fluoro-phenyl)-4-(4-chloro-2-fluoro-phenyl)-4-cyano-5-methyl-pyrrolidine-2-carbonyl]-amino}-piperidine-1-carboxylic acid tert-butyl ester

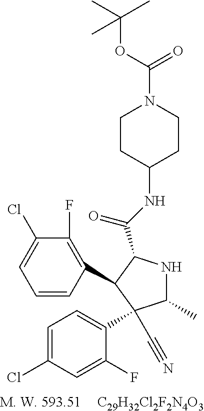

In a manner similar to the method described in Example 1f, rac-(2R,3S,4R,5R)-3-(3-chloro-2-fluoro-phenyl)-4-(4-chloro-2-fluorophenyl)-4-cyano-5-methyl-pyrrolidine-2-carboxylic acid; compound with trifluoro-acetic acid prepared in Example 2d (0.53 g, 1.0 mmol) was reacted with 4-amino-1-Boc-piperidine (0.30 g, 1.5 mmol), HATU (0.57 g, 1.5 mmol) and iPr₂NEt (0.26 mL, 1.5 mmol) in CH₂Cl₂ (5 mL) at room temperature to for 1.5 hours give rac-4-{[(2R,3S,4R,5R)-3-(3-Chloro-2-fluoro-phenyl)-4-(4-chloro-2-fluoro-phenyl)-4-cyano-5-methyl-pyrrolidine-2-carbonyl]-amino}-piperidine-1-carboxylic acid tert-butyl ester as a pink gum (0.60 g, 100% yield). MS (ES⁺) m/z [(M+H)⁺]: 593.

### Example 10b

### Preparation of rac-(2R,3S,4R,5R)-1-acetyl-3-(3-chloro-2-fluoro-phenyl)-4-(4-chloro-2-fluoro-phenyl)-4-cyano-5-methyl-pyrrolidine-2-carboxylic acid (1-acetyl-piperidin-4-yl)-amide

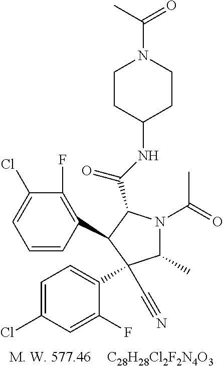

In a manner similar to the method described in Example 2d rac-4-{[(2R,3S,4R,5R)-3-(3-chloro-2-fluoro-phenyl)-4-(4-chloro-2-fluoro-phenyl)-4-cyano-5-methyl-pyrrolidine-2-carbonyl]-amino}-piperidine-1-carboxylic acid tert-butyl ester prepared in Example 10a (0.6 g, 1 mmol) in dichloromethane (10 mL) was reacted with trifluoroacetic acid (3 mL) at room temperature for 0.5 h to give a white solid. This was used without purification.

In a 10 mL round-bottomed flask, the above solid and acetic anhydride (0.06 mL, 0.62 mmol) were combined in CH₂Cl₂ (7 mL) to give a colorless solution. Triethylamine (Fluka, 0.29 mL, 2.0 mmol) was added and the reaction was stirred at room temperature for 0.5 h. Water (3 mL) was added and the reaction mixture was extracted with CH₂Cl₂ (3×8 mL). The organic layers were dried over Na₂SO₄ and concentrated in vacuo. The crude material was purified by flash chromatography (MeOH/CH₂Cl₂ 2% to 6%) to give rac-(2R,3S,4R,5R)-1-acetyl-3-(3-chloro-2-fluoro-phenyl)-4-(4-chloro-2-fluoro-phenyl)-4-cyano-5-methyl-pyrrolidine-2-carboxylic acid (1-acetyl-piperidin-4-yl)-amide as a white solid (98 mg, 40% yield). MS (ES⁺) m/z [(M+H)⁺] 577.

### Example 11a

### Preparation of Intermediate rac-4-{[(2R,3S,4R,5S)-3-(3-chloro-2-fluoro-phenyl)-4-(4-chloro-2-fluoro-phenyl)-4-cyano-5-methyl-pyrrolidine-2-carbonyl]-amino}-piperidine-1-carboxylic acid tert-butyl ester

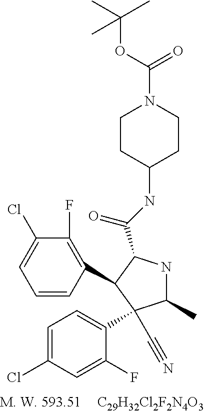

In a manner similar to the method described in Example 1f, rac-(2R,3S,4R,5S)-3-(3-chloro-2-fluoro-phenyl)-4-(4-chloro-2-fluorophenyl)-4-cyano-5-methyl-pyrrolidine-2-carboxylic acid; compound with trifluoro-acetic acid prepared in Example 2e (0.53 g, 1.0 mmol) was reacted with 4-amino-1-Boc-piperidine (0.30 g, 1.5 mmol), HATU (0.57 g, 1.5 mmol) and iPr₂NEt (0.26 mL, 1.5 mmol) in CH₂Cl₂ (5 mL) at room temperature for 1.5 hours to give rac-4-{[(2R,3S,4R,5S)-3-(3-chloro-2-fluoro-phenyl)-4-(4-chloro-2-fluoro-phenyl)-4-cyano-5-methyl-pyrrolidine-2-carbonyl]-amino}-piperidine-1-carboxylic acid tert-butyl ester as a pink gum (0.60 g, 100% yield). MS (ES⁺) m/z [(M+H)⁺]: 593.

### Example 11b

### Preparation of rac-(2R,3S,4R,5S)-1-acetyl-3-(3-chloro-2-fluoro-phenyl)-4-(4-chloro-2-fluoro-phenyl)-4-cyano-5-methyl-pyrrolidine-2-carboxylic acid (1-acetyl-piperidin-4-yl)-amide

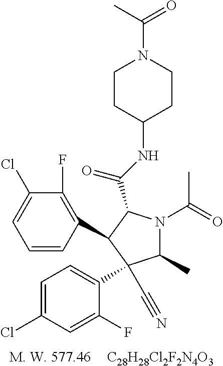

In a manner similar to the method described in Example 2d rac-4-{[(2R,3S,4R,5S)-3-(3-chloro-2-fluoro-phenyl)-4-(4-chloro-2-fluoro-phenyl)-4-cyano-5-methyl-pyrrolidine-2-carbonyl]-amino}-piperidine-1-carboxylic acid tert-butyl ester prepared in Example 11a (0.59 g, 1 mmol) in dichloromethane (10 mL) was reacted with trifluoroacetic acid (3 mL) at room temperature for 0.5 h to give a white solid. This was used without purification.

In a 10 mL round-bottomed flask, the solid (0.2 g, 0.28 mmol) and acetic anhydride (0.036 mL, 0.38 mmol) were combined in CH₂Cl₂ (3 mL) to give a colorless solution. Triethylamine (Fluka, 0.19 mL, 1.4 mmol) was added and the reaction was stirred at room temperature for 0.5 h. Water (3 mL) was added and the reaction mixture was extracted with CH₂Cl₂ (3×8 mL). The organic layers were dried over Na₂SO₄ and concentrated in vacuo. The crude material was purified by flash chromatography (MeOH/CH₂Cl₂ 3% to 6%) to give rac-(2R,3S,4R,5S)-1-acetyl-3-(3-chloro-2-fluoro-phenyl)-4-(4-chloro-2-fluoro-phenyl)-4-cyano-5-methyl-pyrrolidine-2-carboxylic acid (1-acetyl-piperidin-4-yl)-amide as a white solid (25 mg, 16% yield). MS (ES⁺) m/z [(M+H)⁺] 577.

### Example 12a

### Preparation of Intermediate rac-(2R,3S,4R)-3-(3-chloro-2-fluoro-phenyl)-4-(4-chloro2-fluoro-phenyl)-4-cyano-5-methyl-pyrrolidine-2-carboxylic acid tert-butyl ester

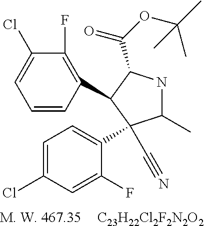

To a solution of eth-(E)-ylideneamino-acetic acid tert-butyl ester in CH₂Cl₂ (100 mL) prepared in Example 2a (34 mmol) was added (Z)-3-(3-chloro-2-fluoro-phenyl)-2-(4-chloro-2-fluoro-phenyl)-acrylonitrile (3.43 g, 11.5 mmol) prepared in Example 2b, AgF (1.12 g, 8.84 mmol), and triethylamine (3.85 mL, 27.63 mmol). The reaction was stirred at room temperature for 18 h. The reaction mixture was filtered through a silica gel pad eluting with CH₂Cl₂ then EtOAc. The filtrate was concentrated to give an oil, which was purified by chromatography (EtOAc/hexanes 10% to 25%) to give (2R,3S,4R,5R)-3-(3-chloro-2-fluoro-phenyl)-4-(4-chloro2-fluoro-phenyl)-4-cyano-5-methyl-pyrrolidine-2-carboxylic acid tert-butyl ester as a white foam (1.79 g, 35% yield). The material was directly used for the next step.

### Example 12b

### Preparation of Intermediate rac-(2R,3S,4R)-3-(3-chloro-2-fluoro-phenyl)-4-(4-chloro-2-fluoro-phenyl)-4-cyano-5-methyl-1-(3-methyl-benzyl)-pyrrolidine-2-carboxylic acid tert-butyl ester

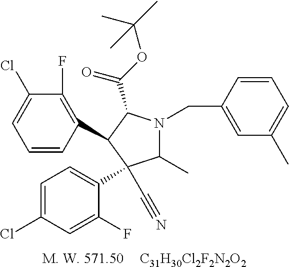

In a 20 mL round-bottomed flask, rac-(2R,3S,4R)-3-(3-chloro-2-fluoro-phenyl)-4-(4-chloro2-fluoro-phenyl)-4-cyano-5-methyl-pyrrolidine-2-carboxylic acid tert-butyl ester prepared in Example 12a (200 mg, 0.43 mmol) was dissolved in DMF (5 ml), 3-methylbenzyl bromide (Aldrich, 0.12 mg, 0.66 mmol) and cesium carbonate (Aldrich) (140 mg, 0.43 mmol) were added. The reaction mixture was stirred at room temperature overnight. The reaction mixture was quenched with water and extracted with EtOAc (3×25 mL). The organic layers were combined, washed with H₂O (1×10 mL), sat NaCl (1×10 mL), dried over Na₂SO₄ and concentrated to give rac-(2R,3S,4R)-3-(3-chloro-2-fluoro-phenyl)-4-(4-chloro-2-fluoro-phenyl)-4-cyano-5-methyl-1-(3-methyl-benzyl)-pyrrolidine-2-carboxylic acid tert-butyl ester as a yellow solid (198 mg, 80% yield). The material was directly used for the next step.

### Example 12c

### Preparation of Intermediate rac-4-{[(2R,3S,4R)-3-(3-chloro-2-fluoro-phenyl)-4-(4-chloro-2-fluoro-phenyl)-4-cyano-5-methyl-1-(3-methyl-benzyl)-pyrrolidine-2-carbonyl]-amino}-piperidine-1-carboxylic acid tert-butyl ester

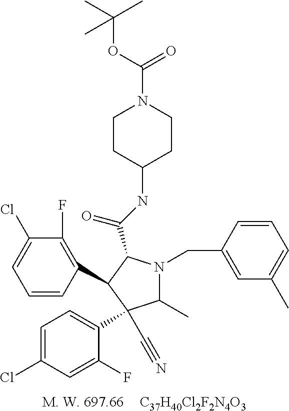

In a manner similar to the method described in Example 2d rac-(2R,3S,4R)-3-(3-chloro-2-fluoro-phenyl)-4-(4-chloro-2-fluoro-phenyl)-4-cyano-1-(3-methyl-benzyl)-5-methyl-pyrrolidine-2-carboxylic acid tert-butyl ester prepared in Example 12b (198 mg, 0.35 mmol) in dichloromethane (5 mL) was reacted with trifluoroacetic acid (2 mL) at room temperature for 24 h to give a residue, which was used directly for the next step.

In a manner similar to the method described in Examples 1f the residue was reacted with 4-amino-1-Boc-piperidine (0.13 g, 0.64 mmol), HATU (0.24 g, 0.64 mmol) and iPr₂NEt (0.18 mL, 0.96 mmol) in CH₂Cl₂ (6 mL) at room temperature for 2 hours to give rac-4-{[(2R,3S,4R)-3-(3-chloro-2-fluoro-phenyl)-4-(4-chloro-2-fluoro-phenyl)-4-cyano-1-(3-methyl-benzyl)-5-methyl-pyrrolidine-2-carbonyl]-amino}-piperidine-1-carboxylic acid tert-butyl ester as a gum (0.21 g, 86% yield). MS (ES⁺) m/z [(M+H)⁺]: 597 (loss of t-boc)

### Example 12d

### Preparation of rac-(2R,3S,4R)-3-(3-chloro-2-fluoro-phenyl)-4-(4-chloro-2-fluoro-phenyl)-4-cyano-1-(3-methyl-benzyl)-5-methyl-pyrrolidine-2-carboxylic acid (1-acetyl-piperidin-4-yl)-amide

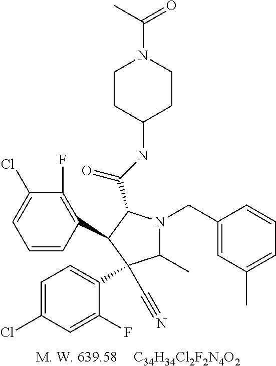

In a manner similar to the method described in Example 2d rac-4-{[(2R,3S,4R)-3-(3-chloro-2-fluoro-phenyl)-4-(4-chloro-2-fluoro-phenyl)-4-cyano-1-(3-methyl-benzyl)-5-methyl-pyrrolidine-2-carbonyl]-amino}-piperidine-1-carboxylic acid tert-butyl ester prepared in Example 12c (0.21 g, 0.30 mmol) in dichloromethane (3 mL) was reacted with trifluoroacetic acid (1 mL) at room temperature for 1 h to give a residue, which was used without purification.

In a manner similar to the method described in Example 10b the residue (100 mg, 0.17 mmol) was reacted with acetic anhydride (35 mg, 0.34 mmol) and triethylamine (Fluka, 0.070 mL, 0.5 mmol) in CH₂Cl₂ (5 mL) at room temperature for 1 hour to give rac-(2R,3S,4R)-3-(3-chloro-2-fluoro-phenyl)-4-(4-chloro-2-fluoro-phenyl)-4-cyano-1-(2-methyl-benzyl)-5-methyl-pyrrolidine-2-carboxylic acid (1-acetyl-piperidin-4-yl)-amide as a light yellow solid (101 mg, 93% yield). MS (ES⁺) m/z [(M+H)⁺] 639

### Example 13

### Preparation of rac-(2R,3S,4R)-3-(3-chloro-2-fluoro-phenyl)-4-(4-chloro-2-fluoro-phenyl)-4-cyano-5-methyl-1-(3-methyl-benzyl)-pyrrolidine-2-carboxylic acid (1-methanesulfonyl-piperidin-4-yl)-amide

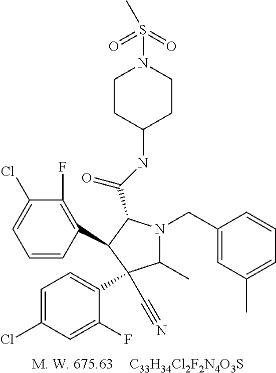

In a manner similar to the method described in Example 2d rac-4-{[(2R,3S,4R)-3-(3-chloro-2-fluoro-phenyl)-4-(4-chloro-2-fluoro-phenyl)-4-cyano-1-(3-methyl-benzyl)-5-methyl-pyrrolidine-2-carbonyl]-amino}-piperidine-1-carboxylic acid tert-butyl ester prepared in Example 12b (0.21 g, 0.30 mmol) in dichloromethane (3 mL) was reacted with trifluoroacetic acid (1 mL) at room temperature for 1 h to give a residue, which was used without purification.

In a manner similar to the method described in Example 10b the residue (100 mg, 0.17 mmol) was reacted with methanesulfonic anhydride (52.3 mg, 0.30 mmol) and triethylamine (Fluka, 0.041 mL, 0.3 mmol) in CH₂Cl₂ (7 mL) at 0° C. for 1 hour to give rac-(2R,3S,4R)-3-(3-chloro-2-fluoro-phenyl)-4-(4-chloro-2-fluoro-phenyl)-4-cyano-5-methyl-1-(3-methyl-benzyl)-pyrrolidine-2-carboxylic acid (1-methanesulfonyl-piperidin-4-yl)-amide as a light yellow solid (105 mg, 91% yield). MS (ES⁺) m/z [(M+H)⁺] 675.

### Example 14a

### Preparation of Intermediate rac-(2R,3S,4R)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid tert-butyl ester

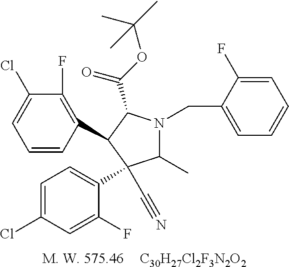

In a 20 mL round-bottomed flask, rac-(2R,3S,4R)-3-(3-chloro-2-fluoro-phenyl)-4-(4-chloro2-fluoro-phenyl)-4-cyano-5-methyl-pyrrolidine-2-carboxylic acid tert-butyl ester prepared in Example 12a (200 mg, 0.43 mmol) was dissolved in DMF (5 ml), 2-fluorobenzyl bromide (Aldrich, 97 mg, 0.51 mmol) and cesium carbonate (Aldrich) (150 mg, 0.47 mmol) were added. The reaction mixture was stirred at room temperature overnight. The reaction mixture was quenched with water and extracted with EtOAc (3×25 mL). The organic layers were combined, washed with H₂O (1×10 mL), sat NaCl (1×10 mL), dried over Na₂SO₄ and concentrated to give a yellow gum (250 mg, 100% yield). MS (ES⁺) m/z [(M+H−C₄H₈)⁺] 519.

### Example 14b

### Preparation of Intermediate rac-(2R,3S,4R)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid

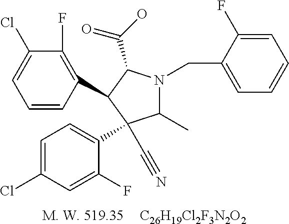

In a manner similar to the method described in Example 2d rac-(2R,3S,4R)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid tert-butyl ester prepared in Example 14a (246 mg, 4.28 mmol) in dichloromethane (6 mL) was reacted with trifluoroacetic acid (3 mL) at room temperature for 48 h to give rac-(2R,3S,4R)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid as a light yellow solid (220 mg g, 99%). MS (ES⁺) m/z [(M+H)⁺] 519

### Example 14c

### Preparation of Intermediate rac-4-{[(2R,3S,4R)-3-(3-chloro-2-fluoro-phenyl)-4-(4-chloro-2-fluoro-phenyl)-4-cyano-1-(2-fluoro-benzyl)-5-methyl-pyrrolidine-2-carbonyl]-amino}-piperidine-1-carboxylic acid tert-butyl ester

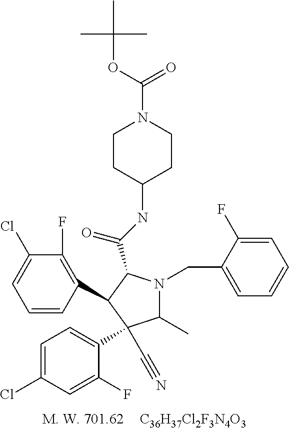

In a manner similar to the method described in Examples 1f rac-4-{[(2R,3S,4R)-3-(3-chloro-2-fluoro-phenyl)-4-(4-chloro-2-fluoro-phenyl)-4-cyano-1-(2-fluoro-benzyl)-5-methyl-pyrrolidine-2-carbonyl]-amino}-piperidine-1-carboxylic acid tert-butyl ester prepared in Example 14b (210 mg, 0.40 mmol) was reacted with 4-amino-1-Boc-piperidine (0.13 g, 0.64 mmol), HATU (0.24 g, 0.64 mmol) and iPr₂NEt (0.12 mL, 0.64 mmol) in CH₂Cl₂ (4 mL) at room temperature to give rac-4-{[(2R,3S,4R)-3-(3-chloro-2-fluoro-phenyl)-4-(4-chloro-2-fluoro-phenyl)-4-cyano-1-(2-fluoro-benzyl)-5-methyl-pyrrolidine-2-carbonyl]-amino}-piperidine-1-carboxylic acid tert-butyl ester as a light yellow foam (0.15 g, 53% yield). MS (ES⁺) m/z [(M+H)⁺]: 701.

### Example 14d

### Preparation of rac-(2R,3S,4R)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid (1-acetyl-piperidin-4-yl)-amide

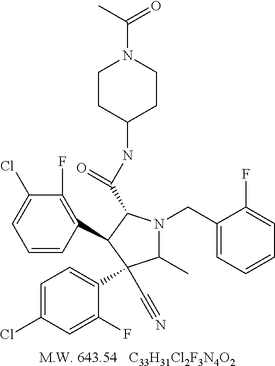

In a manner similar to the method described in Example 2d rac-4-{[(2R,3S,4R,5R)-3-(3-Chloro-2-fluoro-phenyl)-4-(4-chloro-2-fluoro-phenyl)-4-cyano-5-methyl-pyrrolidine-2-carbonyl]-amino}-piperidine-1-carboxylic acid tert-butyl ester prepared in Example 14c (0.158 g, 0.23 mmol) in dichloromethane (3 mL) was reacted with trifluoroacetic acid (1 mL) at room temperature for 0.5 h to give a yellow solid. This was used without purification.

In a manner similar to the method described in Example 10b the yellow solid was reacted with acetic anhydride (0.082 mL, 0.85 mmol) and triethylamine (Fluka, 0.30 mL, 2.14 mmol) in CH₂Cl₂ (3 mL) at room temperature for 20 minutes to give rac-(2R,3S,4R)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid (1-acetyl-piperidin-4-yl)-amide as a white solid (55 mg, 38% yield). MS (ES⁺) m/z [(M+H)⁺] 643.

### Example 15

### Preparation of rac-(2R,3S,4R,5S)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid (1-acetyl-piperidin-4-yl)-amide

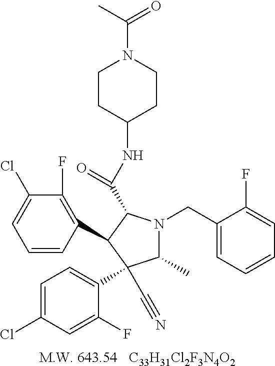

In a manner similar to the method described in Example 2d rac-4-{[(2R,3S,4R,5R)-3-(3-chloro-2-fluoro-phenyl)-4-(4-chloro-2-fluoro-phenyl)-4-cyano-5-methyl-pyrrolidine-2-carbonyl]-amino}-piperidine-1-carboxylic acid tert-butyl ester prepared in Example 14c (0.15 g, 0.21 mmol) in dichloromethane (3 mL) was reacted with trifluoroacetic acid (1 mL) at room temperature for 0.5 h to give a yellow solid. This was used without purification.

In a manner similar to the method described in Example 10b the yellow solid was reacted with acetic anhydride (0.082 mL, 0.85 mmol) and triethylamine (Fluka, 0.30 mL, 2.14 mmol) in CH₂Cl₂ (3 mL) and the products were separated by column chromatography to give rac-(2R,3S,4R,5S)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid (1-acetyl-piperidin-4-yl)-amide as a white solid (24 mg, 18% yield). MS (ES⁺) m/z [(M+H)⁺] 643.

### Example 16

### Preparation of rac-(2R,3S,4R,5R)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid (1-acetyl-piperidin-4-yl)-amide

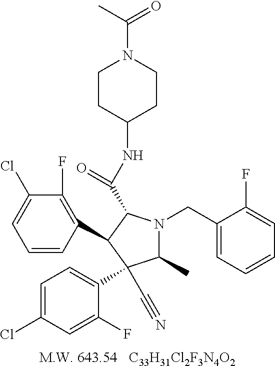

The crude products obtained in Example 14 were separated by column chromatography to give rac-(2R,3S,4R,5R)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid (1-acetyl-piperidin-4-yl)-amide as a white solid (78 mg, 59% yield). MS (ES⁺) m/z [(M+H)⁺] 643

### Example 17a

### Preparation of Intermediate [5-(tert-butyl-dimethyl-silanyloxy)-3,3-dimethyl-pent-(E)-ylideneamino]-acetic acid methyl ester

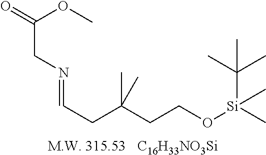

Step A

To the solution of 3,3-dimethylglutaric acid (Aldrich) (5.1 g, 32 mmol) in anhydrous tetrahydrofuran (100 mL) at 0° C. was added a solution of BH₃.THF (1 M, 100 mL, 100 mmol). The reaction mixture was stirred at room temperature for 18 h. Aqueous HCl solution was added, and the mixture was extracted with ethyl acetate. The organic layer was separated, washed with saturated aqueous NaHCO₃ solution, brine, dried over MgSO₄, and concentrated. The residue was purified by chromatography (EtOAc) to give 3,3-dimethyl-pentane-1,5-diol as a colorless oil (1.5 g, 34%).

### Step B

A mixture of 3,3-dimethyl-pentane-1,5-diol (1.5 g, 11 mmol) and imidazole (1.4 g, 20 mmol) in dichloromethane (50 mL) was added tert-butyldimethylchlorosilane (1.7 g, 11 mmol). The reaction mixture was stirred at room temperature for 2 h. Water was added. The organic layer was separated, the aqueous layer was then extracted with dichloromethane. The combined organic layers were washed with brine, dried over MgSO₄, concentrated to give 5-(tert-butyl-dimethyl-silanyloxy)-3,3-dimethyl-pentan-1-ol as a colorless oil (2.7 g, 100%).

### Step C

To a solution of oxalyl chloride (0.97 mL, 11 mmol) (Aldrich) in dichloromethane (20 mL) at −78° C. was added the solution of dimethyl sulfoxide (1.6 mL, 22 mmol) in dichloromethane (5 mL) dropwise. After 5 mins, the solution of 5-(tert-butyl-dimethyl-silanyloxy)-3,3-dimethyl-pentan-1-ol (2.5 g, 10 mmol) in dichloromethane (10 mL) was added dropwise. The reaction mixture was stirred at −78° C. for 15 min. Triethylamine (5 mL, 36 mmol) was added and the reaction mixture was slowly warmed up to room temperature and stirred at room temperature for 45 min. Then water was added. The organic layers were separated, and the aqueous layer was extracted with dichloromethane. The organic layers were combined, washed with 10% of HCl, saturated NaHCO₃, brine, dried over MgSO₄, and concentrated to give 5-(tert-butyl-dimethyl-silanyloxy)-3,3-dimethyl-pentanal as a light yellow oil (Yield: 1.75 g, 71%).

### Step D

In a manner similar to the method described in Example 1a, glycine methyl ester hydrochloride (0.9 g, 7.2 mmol) was reacted with 5-(tert-butyl-dimethyl-silanyloxy)-3,3-dimethyl-pentanal (1.75 g, 7.2 mmol) and triethylamine (1.49 mL, 11 mmol) in CH₂Cl₂ at room temperature for 18 h to give [4-(tert-butyl-dimethyl-silanyloxy)-3,3-dimethyl-but-(E)-ylideneamino]-acetic acid methyl ester as a colorless oil (2.3 g, 100%).

### Example 17b

### Preparation of Intermediate rac-(2R,3S,4R,5S)-5-[4-(tert-tutyl-dimethyl-silanyloxy)-2,2-dimethyl-butyl]-3-(3-chloro-2-fluoro-phenyl)-4-(4-chloro-2-fluoro-phenyl)-4-cyano-pyrrolidine-2-carboxylic acid methyl ester

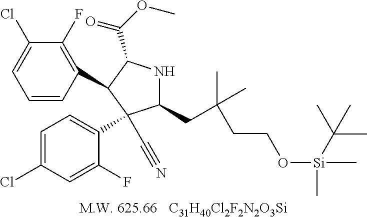

To a solution of [5-(tert-butyl-dimethyl-silanyloxy)-3,3-dimethyl-pent-(E)-ylideneamino]-acetic acid methyl ester (6.4 mmol) and (Z)-3-(3-chloro-2-fluoro-phenyl)-2-(4-chloro-2-fluoro-phenyl)-acrylonitrile (1.58 g, 5.1 mmol) prepared in Example 2b in dichloromethane (100 mL) were added triethylamine (1.8 mL, 13 mmol), and AgF (1 g, 7.8 mmol) sequentially. The mixture was stirred at room temperature for 48 h. The mixture was then quenched with sat. NH₄Cl and extracted with CH₂Cl₂. The organic layer was separated, and aqueous layer was extracted with ethyl acetate. The organic layers were combined, dried over Na₂SO₄, and concentrated. The residue was purified by chromatography (25% EtOAc in hexanes) to give rac-(2R,3S,4R,5S)-5-[4-(tert-tutyl-dimethyl-silanyloxy)-2,2-dimethyl-butyl]-3-(3-chloro-2-fluoro-phenyl)-4-(4-chloro-2-fluoro-phenyl)-4-cyano-pyrrolidine-2-carboxylic acid methyl ester as a yellow gum (1.6 g, 50%).

### Example 17c

### Preparation of Intermediate rac-(2R,3S,4R,5S)-3-(3-chloro-2-fluoro-phenyl)-4-(4-chloro-2-fluoro-phenyl)-4-cyano-5-(4-hydroxy-2,2-dimethyl-butyl)-pyrrolidine-2-carboxylic acid

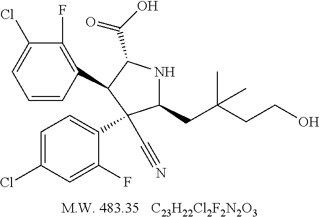

To rac-(2R,3S,4R,5S)-5-[4-(tert-tutyl-dimethyl-silanyloxy)-2,2-dimethyl-butyl]-3-(3-chloro-2-fluoro-phenyl)-4-(4-chloro-2-fluoro-phenyl)-4-cyano-pyrrolidine-2-carboxylic acid methyl ester (0.7 g, 1.1 mmol) in tetrahydrofuran (10 mL) was added tetrahydrofuran solution (1 M, Aldrich) of TBAF (1.34 mL, 1.3 mmol). The reaction mixture was stirred at room temperature for 18 h. The mixture was concentrated, the residue was partitioned between ethyl acetate and water. The organic layer was separated, dried over MgSO₄, and concentrated. The residue was dissolved into tetrahydrofuran (10 mL), and an aqueous solution (1 M) of LiOH (10 mL, 10 mmol) was added. The reaction mixture was stirred at room temperature for 1 h. The “pH” of the mixture was adjusted to ˜4-5 by aqueous HCl solution. The mixture was concentrated. The residue was partitioned between ethyl acetate and water. The organic layer was separated, washed with water, brine, dried over MgSO₄, concentrated to give intermediate rac-(2R,3S,4R,5S)-3-(3-chloro-2-fluoro-phenyl)-4-(4-chloro-2-fluoro-phenyl)-4-cyano-5-(4-hydroxy-2,2-dimethyl-butyl)-pyrrolidine-2-carboxylic acid as a light yellow solid (0.3 g, 54%)

### Example 17d

### Preparation of Intermediate rac-(2R,3S,4R,5S)-3-(3-chloro-2-fluoro-phenyl)-4-(4-chloro-2-fluoro-phenyl)-4-cyano-5-(4-hydroxy-2,2-dimethyl-butyl)-pyrrolidine-2-carboxylic acid [2-((S)-2,2-dimethyl-[1,3]dioxolan-4-yl)-ethyl]-amide

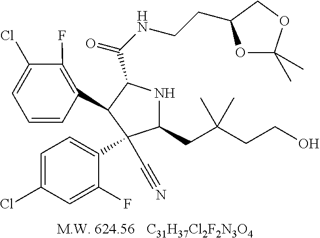

A mixture of rac-(2R,3S,4R,5S)-3-(3-chloro-2-fluoro-phenyl)-4-(4-chloro-2-fluoro-phenyl)-4-cyano-5-(4-hydroxy-2,2-dimethyl-butyl)-pyrrolidine-2-carboxylic acid (0.18 g, 0.36 mmol), 2-((S)-2,2-dimethyl-[1,3]dioxolan-4-yl)-ethylamine (0.16 g, 1 mmol), HATU (0.25 g, 0.65 mmol) and iPr₂NEt (0.07 mL, 0.43 mmol) in CH₂Cl₂ (15 mL) was stirred at room temperature for 20 h. The mixture was then diluted with CH₂Cl₂ and washed with water, brine. The organic phase was separated, filtered and dried over Na₂SO₄. The mixture was then concentrated and the residue was purified by chromatography to give rac-(2R,3S,4R,5S)-3-(3-chloro-2-fluoro-phenyl)-4-(4-chloro-2-fluoro-phenyl)-4-cyano-5-(4-hydroxy-2,2-dimethyl-butyl)-pyrrolidine-2-carboxylic acid [2-((S)-2,2-dimethyl-[1,3]dioxolan-4-yl)-ethyl]-amide as a light yellow gum (0.17 g, 77%).

### Example 17e

### Preparation of Intermediate methanesulfonic acid rac-4-{(2S,3R,4S,5R)-4-(3-chloro-2-fluoro-phenyl)-3-(4-chloro-2-fluoro-phenyl)-3-cyano-5-[2-((S)-2,2-dimethyl-[1,3]dioxolan-4-yl)-ethylcarbamoyl]-pyrrolidin-2-yl}-3,3-dimethyl-butyl ester

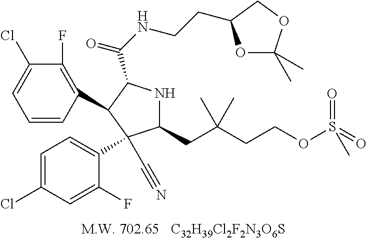

To the solution of rac-(2R,3S,4R,5S)-3-(3-chloro-2-fluoro-phenyl)-4-(4-chloro-2-fluoro-phenyl)-4-cyano-5-(4-hydroxy-2,2-dimethyl-butyl)-pyrrolidine-2-carboxylic acid [2-((S)-2,2-dimethyl-[1,3]dioxolan-4-yl)-ethyl]-amide (0.13 g, 0.21 mmol) and triethylamine (0.058 mL, 0.41 mmol) in dichloromethane (3 mL) at 0° C. was added methanesulfonyl chloride (Aldrich) (0.019 mL, 0.25 mmol). The reaction mixture was stirred at 0° C. for 0.5 h. Water was added. Organic layer was separated, the aqueous layer was extracted with dichlormethane. The combined organic layers were washed with diluted aqueous HCl solution, saturated aqueous NaHCO₃ solution, brine, dried over MgSO₄, and concentrated to give methanesulfonic acid rac-4-{(2S,3R,4S,5R)-4-(3-chloro-2-fluoro-phenyl)-3-(4-chloro-2-fluoro-phenyl)-3-cyano-5-[2-((S)-2,2-dimethyl-[1,3]dioxolan-4-yl)-ethylcarbamoyl]-pyrrolidin-2-yl}-3,3-dimethyl-butyl ester as a yellow gum (0.1 g, 67%).

### Example 17f

### Preparation of Intermediate rac-(1R,2S,3R,8aS)-2-(3-chloro-2-fluoro-phenyl)-1-(4-chloro-2-fluoro-phenyl)-1-cyano-7,7-dimethyl-octahydro-indolizine-3-carboxylic acid [2-((S)-2,2-dimethyl-[1,3]dioxolan-4-yl)-ethyl]-amide

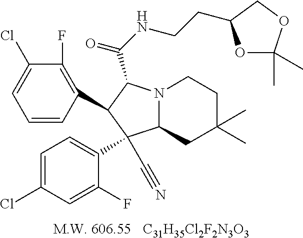

To the solution of methanesulfonic acid rac-4-{(2S,3R,4S,5R)-4-(3-chloro-2-fluoro-phenyl)-3-(4-chloro-2-fluoro-phenyl)-3-cyano-5-[2-((S)-2,2-dimethyl-[1,3]dioxolan-4-yl)-ethylcarbamoyl]-pyrrolidin-2-yl}-3,3-dimethyl-butyl ester (0.1 g, 0.14 mmol) in N,N-dimethylformamide (3 mL) was added Cs₂CO₃ (Aldrich) (0.11 g, 0.35 mmol). The reaction mixture was stirred at room temperature for 18 h. Water was added. The mixture was extracted with ethyl acetate twice. The combined organic layers were washed with water, brine, dried over MgSO₄, and concentrated. The residue was purified by chromatography to give rac-(1R,2S,3R,8aS)-2-(3-chloro-2-fluoro-phenyl)-1-(4-chloro-2-fluoro-phenyl)-1-cyano-7,7-dimethyl-octahydro-indolizine-3-carboxylic acid [2-((S)-2,2-dimethyl-[1,3]dioxolan-4-yl)-ethyl]-amide as a white gum (70 mg, 81%).

### Example 17g

### Preparation of rac-(1R,2S,3R,8aS)-2-(3-chloro-2-fluoro-phenyl)-1-(4-chloro-2-fluoro-phenyl)-1-cyano-7,7-dimethyl-octahydro-indolizine-3-carboxylic acid ((S)-3,4-dihydroxy-butyl)-amide

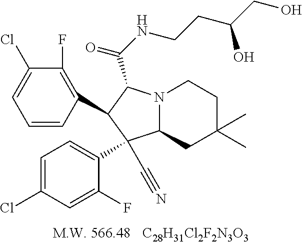

To a solution of rac-(1R,2S,3R,8aS)-2-(3-chloro-2-fluoro-phenyl)-1-(4-chloro-2-fluoro-phenyl)-1-cyano-7,7-dimethyl-octahydro-indolizine-3-carboxylic acid [2-((S)-2,2-dimethyl-[1,3]dioxolan-4-yl)-ethyl]-amide (0.07 g, 0.11 mol) in tetrahydrofuran (9 mL) was added aqueous HCl solution (1 N, 1 mL). The reaction mixture was stirred at room temperature for 2 h, then concentrated. Then the residue was partitioned between ethyl acetate and water. The organic layer was separated, washed with water, aqueous saturated NaHCO₃, brine, dried over MgSO₄, concentrated, dried under reduced pressure to give rac-(1R,2S,3R,8aS)-2-(3-chloro-2-fluoro-phenyl)-1-(4-chloro-2-fluoro-phenyl)-1-cyano-7,7-dimethyl-octahydro-indolizine-3-carboxylic acid ((S)-3,4-dihydroxy-butyl)-amide as a white solid (53 mg, 82%).

HRMS (ES⁺) m/z Calcd for C₂₈H₃₁Cl₂F₂N₃O₃+H [(M+H)⁺]: 566.1784. found: 566.1787.

### Example 18a

### Preparation of Intermediate rac-(2R,3S,4R)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid tert-butyl ester

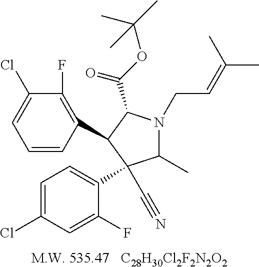

In a manner similar to the method described in Example 12b, rac-(2R,3S,4R)-3-(3-chloro-2-fluoro-phenyl)-4-(4-chloro2-fluoro-phenyl)-4-cyano-5-methyl-pyrrolidine-2-carboxylic acid tert-butyl ester prepared in Example 12a (300 mg, 0.64 mmol) was reacted with 1-bromo-3-methyl-2-butene (Aldrich) (0.143 mg, 0.96 mmol) and cesium carbonate (1.92 mmol) in DMF (5 ml) at room temperature for 4 hours to give a residue, which was used for next step without purification.

### Example 18b

### Preparation of rac-(2R,3S,4R,5R)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid (1-methanesulfonyl-piperidin-4-yl)-amide

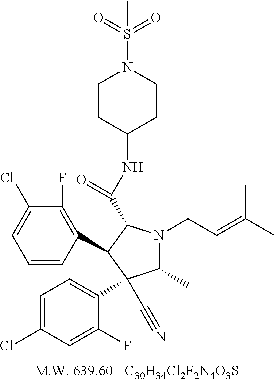

In a manner similar to the method described in Example 2d rac-(2R,3S,4R)-3-(3-chloro-2-fluoro-phenyl)-4-(4-chloro-2-fluoro-phenyl)-4-cyano-1-(3-methyl-but-2-enyl)-5-methyl-pyrrolidine-2-carboxylic acid tert-butyl ester prepared in Example 18a in dichloromethane (5 mL) was reacted with trifluoroacetic acid (2 mL) at room temperature for 24 h to give a foam, which was used directly for the next step.

In a manner similar to the method described in Example 1f the residue (0.21 mmol) was reacted with 1-(methylsulfonyl)piperidine-4-amine (Astginc) (75 mg, 0.42 mmol), HATU (0.1 g, 0.26 mmol) and iPr₂NEt (0.5 mL, 2.8 mmol) in CH₂Cl₂ (5 mL) at room temperature for 2 hours to give rac-(2R,3S,4R,5R)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid (1-methanesulfonyl-piperidin-4-yl)-amide as a solid (44 mg) MS (ES⁺) m/z [(M+H)⁺]: 639.

### Example 19

### Preparation of rac-(2R,3S,4R,5S)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid (1-methanesulfonyl-piperidin-4-yl)-amide

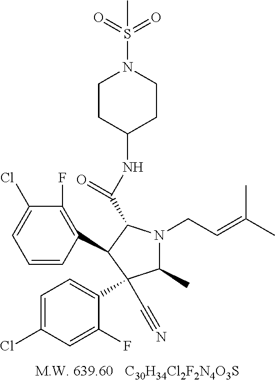

In a manner similar to the method described in Example 2d rac-(2R,3S,4R)-3-(3-chloro-2-fluoro-phenyl)-4-(4-chloro-2-fluoro-phenyl)-4-cyano-1-(3-methyl-but-2-enyl)-5-methyl-pyrrolidine-2-carboxylic acid tert-butyl ester prepared in Example 10a in dichloromethane (5 mL) was reacted with trifluoroacetic acid (2 mL) at room temperature for 24 h to give a foam, which was used directly for the next step.

In a manner similar to the method described in Example 1e the residue (0.21 mmol) was reacted with 1-(methylsulfonyl)piperidine-4-amine (Astginc) (75 mg, 0.42 mmol), HATU (0.1 g, 0.26 mmol) and iPr₂NEt (0.5 mL, 2.8 mmol) in CH₂Cl₂ (5 mL) at room temperature for 2 hours to give rac-(2R,3S,4R,5R)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid (1-methanesulfonyl-piperidin-4-yl)-amide as a solid (0.32 g). MS (ES⁺) m/z [(M+H)⁺]: 639.

### Example 20

### Preparation of (2R,3S,4R)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid (4-methanesulfonylamino-cyclohexyl)-amide

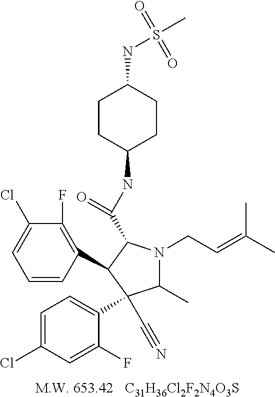

In a manner similar to the method described in Example 2d rac-(2R,3S,4R)-3-(3-chloro-2-fluoro-phenyl)-4-(4-chloro-2-fluoro-phenyl)-4-cyano-1-(3-methyl-but-2-enyl)-5-methyl-pyrrolidine-2-carboxylic acid tert-butyl ester prepared in Example 10a in dichloromethane (5 mL) was reacted with trifluoroacetic acid (2 mL) at room temperature for 24 h to give a foam, which was used directly for the next step.

In a manner similar to the method described in Example 1e the residue (0.21 mmol) was reacted with N-(4-Amino-cyclohexyl)-methanesulfonamide; compound with trifluoro-acetic acid (0.13 g, 0.42 mmol), HATU (0.16 g, 0.42 mmol) and iPr₂NEt (0.5 mL, 2.8 mmol) in CH₂Cl₂ (3 mL) at room temperature for 2 hours to give rac-(2R,3S,4R)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid (4-methanesulfonylamino-cyclohexyl)-amide (93 mg, 68% yield) MS (ES⁺) m/z [(M+H)⁺]: 653.

### Example 21a

### Preparation of Intermediate rac-(2R,3S,4R)-3-(3-chloro-2-fluoro-phenyl)-4-(4-chloro-2-fluoro-phenyl)-4-cyano-1-(3-methoxy-benzyl)-5-methyl-pyrrolidine-2-carboxylic acid tert-butyl ester

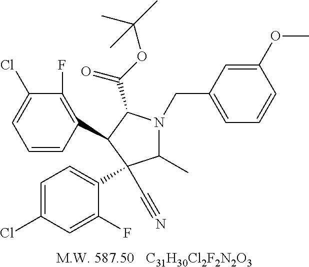

In a manner similar to the method described in Example 12b, rac-(2R,3S,4R)-3-(3-chloro-2-fluoro-phenyl)-4-(4-chloro2-fluoro-phenyl)-4-cyano-5-methyl-pyrrolidine-2-carboxylic acid tert-butyl ester prepared in Example 12a (200 mg, 0.43 mmol) was reacted with 3-methoxybenzyl bromide (Aldrich, 97 mg, 0.51 mmol) and cesium carbonate (140 mg, 0.43 mmol) in DMF (5 ml) at room temperature for 72 hours to give a yellow gum (250 mg, 100% yield).

MS (ES⁺) m/z [(M+H−C₄H₈)⁺] 531.

### Example 21b

### Preparation of Intermediate rac-(2R,3S,4R)-3-(3-chloro-2-fluoro-phenyl)-4-(4-chloro-2-fluoro-phenyl)-4-cyano-1-(3-methoxy-benzyl)-5-methyl-pyrrolidine-2-carboxylic acid

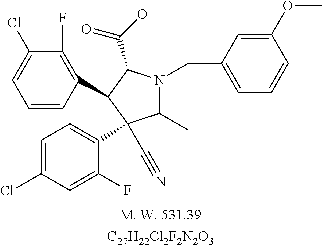

In a manner similar to the method described in Example 2d rac-(2R,3S,4R)-3-(3-chloro-2-fluoro-phenyl)-4-(4-chloro-2-fluoro-phenyl)-4-cyano-1-(3-methoxy-benzyl)-5-methyl-pyrrolidine-2-carboxylic acid tert-butyl ester prepared in Example 21a (246 mg, 4.28 mmol) in dichloromethane (6 mL) was reacted with trifluoroacetic acid (3 mL) at room temperature for 48 h to give rac-(2R,3S,4R)-3-(3-chloro-2-fluoro-phenyl)-4-(4-chloro-2-fluoro-phenyl)-4-cyano-1-(3-methoxy-benzyl)-5-methyl-pyrrolidine-2-carboxylic acid as a light yellow gum (221 mg g, 99%). MS (ES⁺) m/z [(M+H)⁺] 531.

### Example 21c

### Preparation of Intermediate rac-4-{[(2R,3S,4R)-3-(3-chloro-2-fluoro-phenyl)-4-(4-chloro-2-fluoro-phenyl)-4-cyano-1-(3-methoxy-benzyl)-5-methyl-pyrrolidine-2-carbonyl]-amino}-piperidine-1-carboxylic acid tert-butyl ester

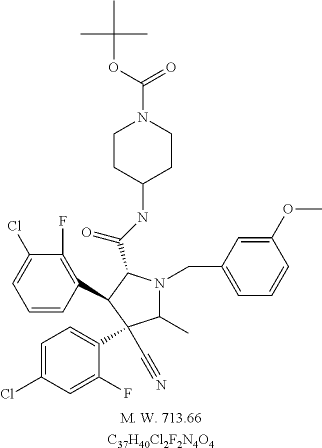

In a manner similar to the method described in Examples 1f rac-4-{[(2R,3S,4R)-3-(3-chloro-2-fluoro-phenyl)-4-(4-chloro-2-fluoro-phenyl)-4-cyano-1-(3-methoxy-benzyl)-5-methyl-pyrrolidine-2-carbonyl]-amino}-piperidine-1-carboxylic acid tert-butyl ester prepared in Example 21b (215 mg, 0.40 mmol) was reacted with 4-amino-1-Boc-piperidine (0.13 g, 0.64 mmol), HATU (0.24 g, 0.64 mmol) and iPr₂NEt (0.18 mL, 0.96 mmol) in CH₂Cl₂ (4 mL) at room temperature for 2 hours to give rac-4-{[(2R,3S,4R)-3-(3-chloro-2-fluoro-phenyl)-4-(4-chloro-2-fluoro-phenyl)-4-cyano-1-(3-methoxy-benzyl)-5-methyl-pyrrolidine-2-carbonyl]-amino}-piperidine-1-carboxylic acid tert-butyl ester as a brownish foam (0.25 g, 87% yield). MS (ES⁺) m/z [(M+H)⁺]: 713.

### Example 21d

### Preparation of rac-(2R,3S,4R)-3-(3-chloro-2-fluoro-phenyl)-4-(4-chloro-2-fluoro-phenyl)-4-cyano-1-(3-methoxy-benzyl)-5-methyl-pyrrolidine-2-carboxylic acid (1-acetyl-piperidin-4-yl)-amide

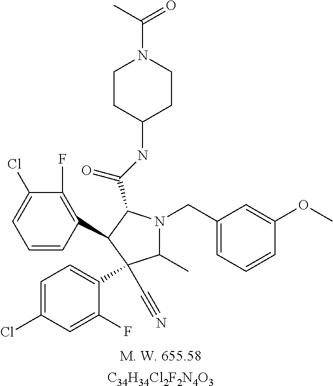

In a manner similar to the method described in Example 2d rac-4-{[(2R,3S,4R)-3-(3-chloro-2-fluoro-phenyl)-4-(4-chloro-2-fluoro-phenyl)-4-cyano-1-(3-methoxy-benzyl)-5-methyl-pyrrolidine-2-carbonyl]-amino}-piperidine-1-carboxylic acid tert-butyl ester prepared in Example 21c (0.25 g, 0.35 mmol) in dichloromethane (3 mL) was reacted with trifluoroacetic acid (1 mL) at room temperature for 0.5 h to give an off white solid. This was used without purification.

In a manner similar to the method described in Example 2g the off-white solid was reacted with acetic anhydride (0.082 mL, 0.85 mmol) and triethylamine (Fluka, 0.30 mL, 2.14 mmol) in CH₂Cl₂ (3 mL) to give rac-(2R,3S,4R)-3-(3-chloro-2-fluoro-phenyl)-4-(4-chloro-2-fluoro-phenyl)-4-cyano-1-(3-methoxy-benzyl)-5-methyl-pyrrolidine-2-carboxylic acid (1-acetyl-piperidin-4-yl)-amide as a white foam (98 mg, 43% yield). MS (ES⁺) m/z [(M+H)⁺] 655

### Example 22a

### Preparation of Intermediate rac-(2R,3S,4R)-3-(3-chloro-2-fluoro-phenyl)-4-(4-chloro-2-fluoro-phenyl)-4-cyano-1-(2-chloro-benzyl)-5-methyl-pyrrolidine-2-carboxylic acid tert-butyl ester

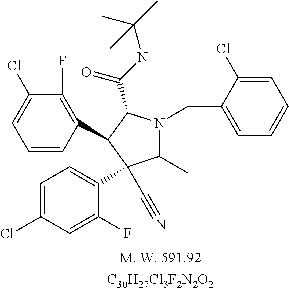

In a manner similar to the method described in Example 12b, rac-(2R,3S,4R)-3-(3-chloro-2-fluoro-phenyl)-4-(4-chloro2-fluoro-phenyl)-4-cyano-5-methyl-pyrrolidine-2-carboxylic acid tert-butyl ester prepared in Example 12a (200 mg, 0.43 mmol) was reacted with 2-chlorobenzyl bromide (Aldrich, 0.067 mL, 0.51 mmol) and cesium carbonate (140 mg, 0.43 mmol) in DMF (3 ml) at room temperature for 72 hours to give a yellow gum (246 mg, 97% yield). MS (ES⁺) m/z [(M+H−C₄H₈)⁺] 535.

### Example 22b

### Preparation of Intermediate rac-(2R,3S,4R)-3-(3-chloro-2-fluoro-phenyl)-4-(4-chloro-2-fluoro-phenyl)-4-cyano-1-(2-chloro-benzyl)-5-methyl-pyrrolidine-2-carboxylic acid

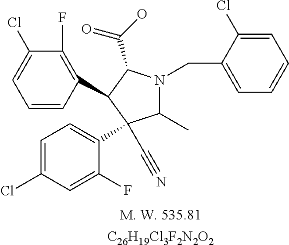

In a manner similar to the method described in Example 2c rac-(2R,3S,4R)-3-(3-chloro-2-fluoro-phenyl)-4-(4-chloro-2-fluoro-phenyl)-4-cyano-1-(2-chloro-benzyl)-5-methyl-pyrrolidine-2-carboxylic acid tert-butyl ester prepared in Example 22a (240 mg, 4.05 mmol) in dichloromethane (6 mL) was reacted with trifluoroacetic acid (3 mL) at room temperature for 48 h to give rac-(2R,3S,4R)-3-(3-Chloro-2-fluoro-phenyl)-4-(4-chloro-2-fluoro-phenyl)-4-cyano-1-(2-chloro-benzyl)-5-methyl-pyrrolidine-2-carboxylic acid as a brownish gum (220 mg, 100%). MS (ES⁺) m/z [(M+H)⁺] 535.

### Example 22c

### Preparation of Intermediate rac-4-{[(2R,3S,4R)-3-(3-chloro-2-fluoro-phenyl)-4-(4-chloro-2-fluoro-phenyl)-4-cyano-1-(2-chloro-benzyl)-5-methyl-pyrrolidine-2-carbonyl]-amino}-piperidine-1-carboxylic acid tert-butyl ester

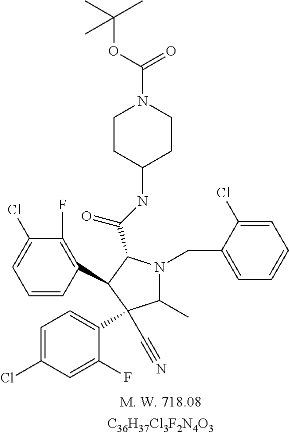

In a manner similar to the method described in Examples 1e rac-4-{[(2R,3S,4R)-3-(3-Chloro-2-fluoro-phenyl)-4-(4-chloro-2-fluoro-phenyl)-4-cyano-1-(2-chloro-benzyl)-5-methyl-pyrrolidine-2-carbonyl]-amino}-piperidine-1-carboxylic acid tert-butyl ester prepared in Example 22b (215 mg, 0.40 mmol) was reacted with 4-amino-1-Boc-piperidine (0.13 g, 0.64 mmol), HATU (0.24 g, 0.64 mmol) and iPr₂NEt (0.18 mL, 0.96 mmol) in CH₂Cl₂ (4 mL) at room temperature for 2 hours to give rac-4-{[(2R,3S,4R)-3-(3-chloro-2-fluoro-phenyl)-4-(4-chloro-2-fluoro-phenyl)-4-cyano-1-(2-chloro-benzyl)-5-methyl-pyrrolidine-2-carbonyl]-amino}-piperidine-1-carboxylic acid tert-butyl ester as a brownish foam (0.26 g, 90% yield). MS (ES⁺) m/z [(M+H)⁺]: 717.

### Example 22d

### Preparation of rac-(2R,3S,4R)-3-(3-chloro-2-fluoro-phenyl)-4-(4-chloro-2-fluoro-phenyl)-4-cyano-1-(2-chloro-benzyl)-5-methyl-pyrrolidine-2-carboxylic acid (1-acetyl-piperidin-4-yl)-amide

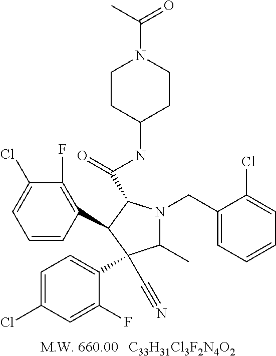

In a manner similar to the method described in Example 2d rac-4-{[(2R,3S,4R)-3-(3-Chloro-2-fluoro-phenyl)-4-(4-chloro-2-fluoro-phenyl)-4-cyano-1-(2-chloro-benzyl)-5-methyl-pyrrolidine-2-carbonyl]-amino}-piperidine-1-carboxylic acid tert-butyl ester prepared in Example 22c (0.26 g, 0.36 mmol) in dichloromethane (3 mL) was reacted with trifluoroacetic acid (1 mL) at room temperature for 0.5 h to give an off white solid. This was used without purification.

In a manner similar to the method described in Example 11b the off white solid was reacted with acetic anhydride (0.082 mL, 0.85 mmol) and triethylamine (Fluka, 0.30 mL, 2.14 mmol) in CH₂Cl₂ (3 mL) to give rac-(2R,3S,4R)-3-(3-Chloro-2-fluoro-phenyl)-4-(4-chloro-2-fluoro-phenyl)-4-cyano-1-(2-chloro-benzyl)-5-methyl-pyrrolidine-2-carboxylic acid (1-acetyl-piperidin-4-yl)-amide as a white foam (50 mg, 21% yield). MS (ES⁺) m/z [(M+H)⁺] 659.

### Example 23a

### Preparation of Intermediate rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid tert-butyl ester

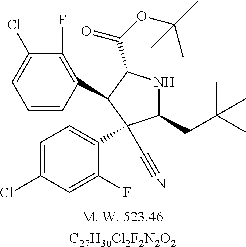

In a manner similar to the method described in Example 1c, [3-methyl-but-(E)-ylideneamino]-acetic acid tert-butyl ester prepared in Example 1a (2.3 g, 11 mmol) was reacted with (Z)-3-(3-chloro-2-fluoro-phenyl)-2-(4-chloro-2-fluoro-phenyl)-acrylonitrile (2.5 g, 8.0 mmol) prepared in Example 2b, AgF (0.7 g, 5.5 mmol), and triethylamine (2.9 g, 29.0 mmol) in dichloromethane (200 mL) at room temperature for 18 h to give rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid tert-butyl ester as a white foam (3.0 g, 71.8%).

### Example 23b

### Preparation of Intermediate rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid trifluoroacetic acid

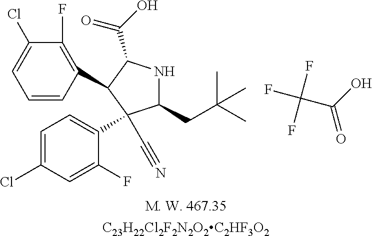

To a solution of rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid tert-butyl ester prepared in Example 22a (0.4 g, 0.8 mmol) in dichloromethane (10 mL) was added trifluoroacetic acid (3 mL). The reaction mixture was stirred at room temperature for 18 h, and concentrated. The residue was then triturated with ethyl ether hexanes, concentrated, dried under reduced pressure to give rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid trifluoroacetic acid as a white solid (0.5 g, 100%).

HRMS (ES⁺) m/z Calcd for C₂₃H₂₂Cl₂F₂N₂O₂+H [(M+H)⁺]: 467.1099. found: 467.1098. Chiral (2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid was obtained by chiral SFC.

### Example 23c

### Preparation rac-(2R,3S,4R,5S)-1-butyl-3-(3-chloro-2-fluoro-phenyl)-4-(4-chloro-2-fluoro-phenyl)-4-cyano-5-(2,2-dimethyl-propyl)-pyrrolidine-2-carboxylic acid [1-(1-butyl-4-hydroxy-piperidin-4-ylmethyl)-1H-pyrazol-3-yl]-amide

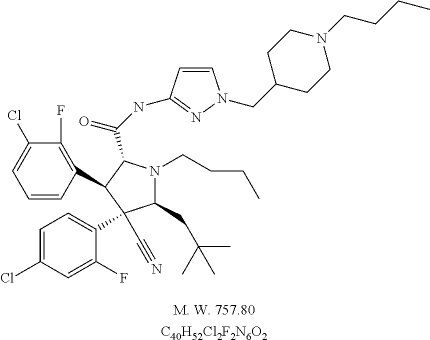

### Step 1. Preparation of 4-(3-amino-pyrazol-1-ylmethyl)-4-hydroxy-piperidine-1-carboxylic acid tert-butyl ester

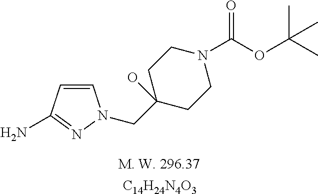

A mixture of 3-nitro-1H-pyrazole (226 mg, 2.0 mmol), 1-oxa-6-aza-spiro[2.5]octane-6-carboxylic acid tert-butyl ester (500 mg, 2.34 mmol), potassium carbonate (345 mg, 2.5 mmol) in DMF (3 mL) was stirred at 80° C. for 1 h. The mixture was then diluted with water and ethyl acetate. The organic phase was separated, dried over Na₂SO₄ and filtered. The mixture was then concentrated to afford 4-hydroxy-4-(3-nitro-pyrazol-1-ylmethyl)-piperidine-1-carboxylic acid tert-butyl ester (0.83 g), which was taken directly to the hydrogenation conditions.

The crude 4-hydroxy-4-(3-nitro-pyrazol-1-ylmethyl)-piperidine-1-carboxylic acid tert-butyl ester was dissolved in ethyl acetate and ethanol (1:1, 60 mL) and slowly (via glass funnel) added to a cooled (−78° C.) Parr bottle containing 10% Pd/C (0.11 g). The bottle was subjected to hydrogen gas (20 psi) and shaken under Parr Apparatus conditions for 1 h. The mixture was then filtered using a Buchner funnel with a glass membrane filter paper (Whatman GF/F) and filter flask, with care so the catalyst is kept moist with solvent rinses [water was added later to catalyst for safe disposal/recycling]. The product is concentrated under reduced pressure to afford 4-(3-amino-pyrazol-1-ylmethyl)-4-hydroxy-piperidine-1-carboxylic acid tert-butyl ester (0.75 g, >100%).

### Step 2. Preparation of rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid [1-(4-hydroxy-piperidin-4-ylmethyl)-1H-pyrazol-3-yl]-amide

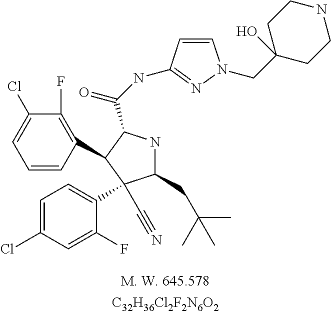

A mixture of rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (500 mg, 1.0 mmol), 4-(3-amino-pyrazol-1-ylmethyl)-4-hydroxy-piperidine-1-carboxylic acid tert-butyl ester (592 mg, 2.0 mmol), 2-(7-azabenzotriazol-1-yl)-n,n,n′,n′-tetramethyluronium hexafluorophosphate (HATU, 593 mg, 1.5 mmol) and iPr₂NEt (0.718 mL, 4 mmol) in CH₂Cl₂ (50 mL) was stirred at 25° C. overnight. The mixture was then diluted with CH₂Cl₂ and washed with water, brine. The organic phase was separated, dried over Na₂SO₄ and filtered. The mixture was then concentrated and purified flash column chromatography (1-100% ethyl acetate/heptane) to afford rac-4-(3-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-pyrazol-1-ylmethyl)-4-hydroxy-piperidine-1-carboxylic acid tert-butyl ester as a white powder (480 mg, 64.4%).

The compound was taken directly to the deprotection step by dissolving in a solution of 30% TFA in dichloromethane (3 mL) and stirring at room temperature for 3 hours. The mixture was diluted with NaHCO₃ (s) and dichloromethane and the organic phase was separated then concentrated under reduced pressure to afford an oil that was purified via triteration with ethyl acetate and heptane to afford a white foam rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid [1-(4-hydroxy-piperidin-4-ylmethyl)-1H-pyrazol-3-yl]-amide (370 mg, 64.3%). HRMS (ES⁺) m/z Calcd for C₃₂H₃₆Cl₂F₂N₆O₂+H [(M+H)⁺]: 645.2318. found: 645.2315.

Step 3. A mixture of rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid [1-(4-hydroxy-piperidin-4-ylmethyl)-1H-pyrazol-3-yl]-amide (40 mg, 0.062 mmol) and butyraldehyde (0.2 mL, 2.2 mmol) was dissolved in THF (2 mL) and dichloromethane (2 mL), stirred at room temperature for 15 minutes, then sodium triacetoxyborohydride (240 mg, 1.13 mmol) was added and stirred overnight at room temperature. The mixture was diluted with 0.1 N NaOH and extracted with ethyl acetate. The organic phase was separated then concentrated under reduced pressure to afford the crude oil that was purified by RP-HPLC (20%-95% acetonitrile/water) to afford an off-white powder rac (2R,3S,4R,5S)-1-butyl-3-(3-chloro-2-fluoro-phenyl)-4-(4-chloro-2-fluoro-phenyl)-4-cyano-5-(2,2-dimethyl-propyl)-pyrrolidine-2-carboxylic acid [1-(1-butyl-4-hydroxy-piperidin-4-ylmethyl)-1H-pyrazol-3-yl]-amide (26.8 mg, 57.0%). HRMS (ES⁺) m/z Calcd for C₄₀H₅₂Cl₂F₂N₆O₂+H [(M+H)⁺]: 757.3570. found: 757.3577.

### Example 24

### Preparation of rac-propionic acid 4-(3-{[(2R,3S,4R,5S)-3-(3-chloro-2-fluoro-phenyl)-4-(4-chloro-2-fluoro-phenyl)-4-cyano-5-(2,2-dimethyl-propyl)-1-propionyl-pyrrolidine-2-carbonyl]-amino}-pyrazol-1-ylmethyl)-1-propionyl-piperidin-4-yl ester

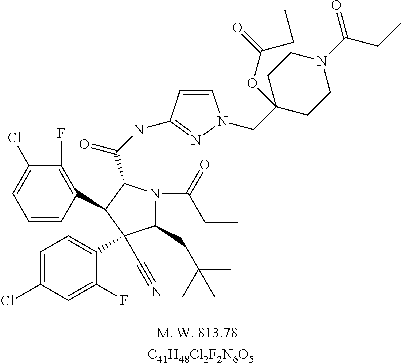

A mixture of rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid [1-(4-hydroxy-piperidin-4-ylmethyl)-1H-pyrazol-3-yl]-amide (40 mg, 0.062 mmol) and diisopropylethylamine (0.1 mL, 0.6 mmol) was dissolved in THF (2 mL) and dichloromethane (2 mL), then propionyl chloride (0.2 mL, 2.3 mmol) was stirred at room temperature overnight. The reaction mixture was concentrated under reduced pressure to afford the crude oil that was purified by RP-HPLC (20%-95% acetonitrile/water) to afford an off-white solid rac-propionic acid 4-(3-{[(2R,3S,4R,5S)-3-(3-chloro-2-fluoro-phenyl)-4-(4-chloro-2-fluoro-phenyl)-4-cyano-5-(2,2-dimethyl-propyl)-1-propionyl-pyrrolidine-2-carbonyl]-amino}-pyrazol-1-ylmethyl)-1-propionyl-piperidin-4-yl ester (18.6 mg, 37.0%). HRMS (ES⁺) m/z Calcd for C₄₁H₄₈Cl₂F₂N₆O₅+H [(M+H)⁺]: 813.3104. found: 813.3106.

### Example 25a

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester

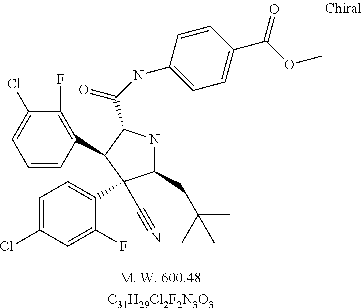

Reactants chiral (2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (300 mg, 0.642 mmol) and 2-(7-azabenzotriazol-1-yl)-n,n,n′,n′-tetramethyluronium hexafluorophosphate (HATU, 317 mg, 0.835 mmol) were added portion wise (3×, 30 min interval) to a solution of 4-amino-benzoic acid methyl ester (0.291 g, 1.93 mmol) and diisopropylethyl amine (0.785 mL, 0.581 g, 4.49 mmol) in CH₂Cl₂ (20 mL) and was stirred at 25° C. for 14 h. The mixture was then diluted with CH₂Cl₂ and washed with water. The organic phase was separated, then concentrated under reduced pressure and purified by column chromatography (23 g Supelco, 0-40% Ethyl acetate/heptane) to afford chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester

(200 mg, 51.9%) as an white solid which was used in the next step without further purification. LCMS [(M+H)⁺]: calcd for C₃₁H₂₉Cl₂F₂N₃O₃: 599.16. found: 600.1.

### Example 25b

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-benzoic acid

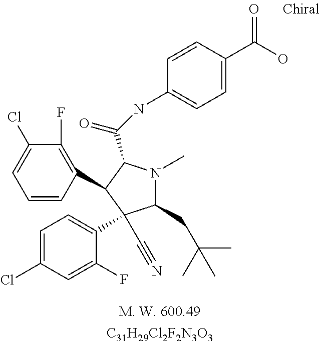

In a manner similar to Example 1d chiral 4-{[(2R,3S,4R,5S)-3-(3-chloro-2-fluoro-phenyl)-4-(4-chloro-2-fluoro-phenyl)-4-cyano-5-(2,2-dimethyl-propyl)pyrrolidine-2-carbonyl]-amino}-benzoic acid methyl ester (0.2 g, 0.333 mmol) was reacted with paraformaldehyde (excess) and sodium triacetoxyborohydride (excess) to afford crude chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-benzoic acid methyl ester as a colorless foam that was taken directly to hydrolysis conditions in THF (6 mL) and methanol (2 mL), followed by 2N LiOH (2 mL). The reaction mixture was stirred at room temperature for 5 hours. The mixture was diluted with water and extracted with ethyl acetate (2×). The organic phase was separated, then concentrated under reduced pressure and purified by RP-HPLC (30-95% acetonitrile/water) to afford the trifluoroacetate salt, which was neutralized with NaHCO₃ (s) and extracted with ethyl acetate/iPA to afford chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-benzoic acid as a white solid (98.9 mg, two steps 49.6% overall). HRMS (ES⁺) m/z Calcd for C₃₁H₂₉Cl₂F₂N₃O₃+H [(M+H)⁺]: 600.1627. found: 600.1622.

### Example 26a

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-fluoro-benzoic acid methyl ester

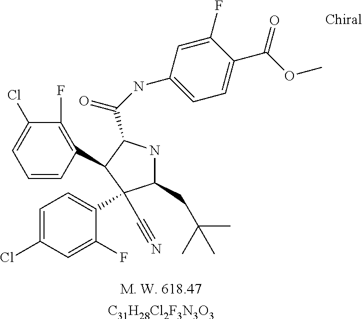

Reactants chiral (2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (300 mg, 0.642 mmol) and 2-(7-azabenzotriazol-1-yl)-n,n,n′,n′-tetramethyluronium hexafluorophosphate (HATU, 317 mg, 0.835 mmol) were added portion wise (3×, 30 min interval) to a solution of 4-amino-2-fluoro-benzoic acid methyl ester (0.500 g, 2.96 mmol) and diisopropylethyl amine (1.35 mL, 0.996 g, 7.7 mmol) in CH₂Cl₂ (20 mL) and was stirred @ 25° C. for 14 h. The mixture was then diluted with CH₂Cl₂ and washed with water. The organic phase was separated, then concentrated under reduced pressure and purified by RP-HPLC column chromatography (30-95% acetonitrile/water) to afford chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-fluoro-benzoic acid methyl ester (100.0 mg, 25.2%) as an off-white solid which was used in the next step without further purification.

### Example 26b

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-fluoro-benzoic acid trifluoroacetate salt

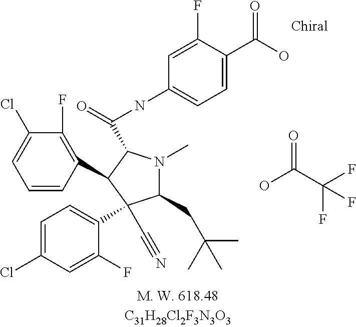

In a manner similar to Example 1d chiral 4-{[(2R,3S,4R,5S)-3-(3-chloro-2-fluoro-phenyl)-4-(4-chloro-2-fluoro-phenyl)-4-cyano-5-(2,2-dimethyl-propyl)pyrrolidine-2-carbonyl]-amino}-2-fluoro-benzoic acid methyl ester (0.1 g, 0.162 mmol) was reacted with paraformaldehyde (excess) and sodium triacetoxyborohydride (excess) to afford crude chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-fluoro-benzoic acid methyl ester that was taken directly to hydrolysis conditions in THF (9 mL) and methanol (3 mL), followed by 2N LiOH (3 mL). The reaction mixture was stirred at room temperature for 14 hours. The mixture was diluted with water and extracted with ethyl acetate (2×). The organic phase was separated, then concentrated under reduced pressure to afford the crude solid. RP-HPLC purification (40-95% acetonitrile/water) to afford chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-fluoro-benzoic acid trifluoroacetate salt as an off-white powder (22 mg, 18.5% over two steps). HRMS (ES⁺) m/z Calcd for C₃₁H₂₈Cl₂F₃N₃O₃+H [(M+H)⁺]: 618.1533. found: 618.1534.

### Example 27a

### Preparation of chiral 2-(4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-phenyl)-2-methyl-propionic acid methyl ester

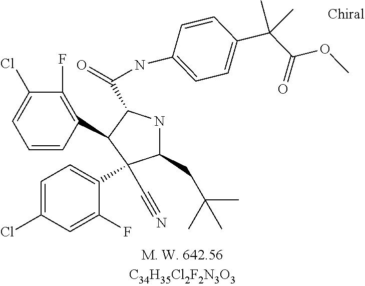

Reactants chiral (2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (300 mg, 0.642 mmol) and 2-(7-azabenzotriazol-1-yl)-n,n,n′,n′-tetramethyluronium hexafluorophosphate (HATU, 317 mg, 0.835 mmol) were added portion wise (3×, 30 min interval) to a solution of 2-(4-amino-phenyl)-2-methyl-propionic acid methyl ester (0.164 g, 0.849 mmol) and diisopropylethyl amine (0.317 g, 0.5 mL, 2.86 mmol) in CH₂Cl₂ (20 mL) and was stirred at 25° C. for 5 h. Additional methyl 2-(4-aminophenyl)-2-methylpropanoate (0.150 g, 0.776 mmol) was added and stirred overnight at rt. The mixture was then diluted with CH₂Cl₂ and washed with water. The organic phase was separated, then concentrated under reduced pressure and purified by column chromatography (80 g Analogix, 0-100% ethyl acetate/heptane) to afford chiral 2-(4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-phenyl)-2-methyl-propionic acid methyl ester (390.0 mg, 94.5%) as an off-white solid. LCMS [(M+H)⁺]: calcd for C₃₄H₃₅Cl₂F₂N₃O₃: 641.2. found: 642.1.

Taken directly on to reductive amination reactions.

### Example 27b

### Preparation of chiral 2-(4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]amino}-phenyl)-2-methyl-propionic acid

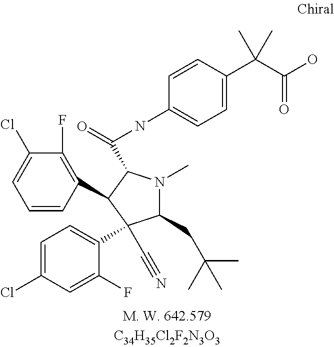

In a manner similar to Example 1d chiral 2-(4-{[(2R,3S,4R,5S)-3-(3-chloro-2-fluoro-phenyl)-4-(4-chloro-2-fluoro-phenyl)-4-cyano-5-(2,2-dimethyl-propyl)pyrrolidine-2-carbonyl]-amino}-phenyl)-2-methyl-propionic acid methyl ester (0.125 g, 0.195 mmol) was reacted with paraformaldehyde (excess) and sodium triacetoxyborohydride (excess) to afford crude chiral 2-(4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]amino}-phenyl)-2-methyl-propionic acid methyl ester that was taken directly to hydrolysis conditions in THF (9 mL) and methanol (3 mL), followed by 2N LiOH (3 mL). The reaction mixture was heated to 60° C. for 8 hours. The mixture was diluted with water and extracted with ethyl acetate (2×). The organic phase was separated, then concentrated under reduced pressure to afford a residue that was taken up in ethyl acetate with a little heptane and precipitated with nitrogen aspiration to afford an off-white solid chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-fluoro-benzoic acid (71.4 mg, 57.0% over two steps). HRMS (ES⁺) m/z Calcd for C₃₄H₃₅Cl₂F₂N₃O₃+H [(M+H)⁺]: 642.2097. found: 642.2094.

### Example 28a

### Preparation of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester

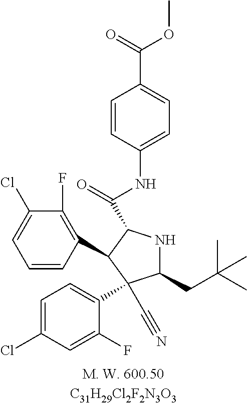

In a manner similar to the method described in Examples 1e, rac-(2R,3S,4R,5S)-3-(5-chloro-2-fluoro-phenyl)-4-(4-chloro-2-fluoro-phenyl)-4-cyano-5-(2,2-dimethyl-propyl)-pyrrolidine-2-carboxylic acid trifluoroacetic acid prepared in Example 26c (0.5 g, 0.89 mmol) was reacted with methyl 4-aminobenzoate (0.24 g, 1.6 mmol), HATU (0.61 g, 1.6 mmol) and iPr₂NEt (0.39 mL, 2.2 mmol) in CH₂Cl₂ at room temperature to give rac-4-{[(2R,3S,4R,5S)-3-(5-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester as a white solid (0.14 g, 27%). HRMS (ES⁺) m/z Calcd for C₃₁H₂₉Cl₂F₂N₃O₃+H [(M+H)⁺]: 600.1627. found: 600.1626.

### Example 28b

### Preparation of rac 4-({(2R,3S,4R,5S)-3-(3-chloro-2-fluoro-phenyl)-4-(4-chloro-2-fluoro-phenyl)-4-cyano-5-(2,2-dimethyl-propyl)-1-[2-(2-fluoro-phenyl)-ethyl]-pyrrolidine-2-carbonyl}-amino)-benzoic acid methyl ester

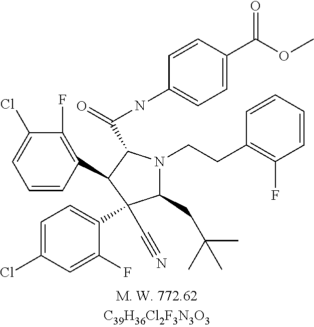

A mixture of racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester (85 mg, 0.142 mmol) was dissolved in acetic acid (2 mL) then 2-(2-fluorophenyl)acetaldehyde (ASDI Intermediates, 0.1 g, 0.724 mmol) was added and stirred 15 min at rt. Then sodium triacetoxyborohydride (Fluka, 0.2 g, 0.944 mmol) was added in two portions, 1 h interval. The reaction mixture was stirred an additional 3 h. The reaction was diluted with ethyl acetate and extracted with 0.1 N NaOH then washed with water. The organic layer was separated and concentrated under reduced pressure to afford crude product that was purified by RP-HPLC (40-95% acetonitrile/water) to afford 4-({(2R,3S,4R,5S)-3-(3-chloro-2-fluoro-phenyl)-4-(4-chloro-2-fluoro-phenyl)-4-cyano-5-(2,2-dimethyl-propyl)-1-[2-(2-fluoro-phenyl)-ethyl]-pyrrolidine-2-carbonyl}-amino)-benzoic acid methyl ester as an off-white solid (15.4 mg, 15.1%). HRMS (ES⁺) m/z Calcd for C₃₉H₃₆Cl₂F₃N₃O₃+H [(M+H)⁺]: 722.2159. found: 722.2164.

### Example 29

### Preparation of rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-benzoic acid methyl ester

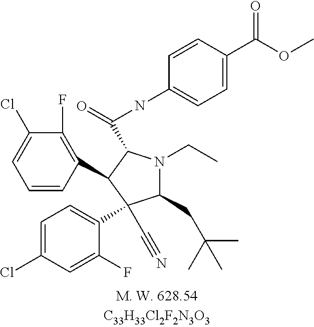

A mixture of racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester (85 mg, 0.142 mmol) was dissolved in acetic acid (2 mL) then acetaldehyde (78 mg, 0.1 mL, 1.7 mmol) was added and stirred at 25° C. After 15 minutes sodium triacetoxyborohydride (Fluka, 0.2 g, 0.944 mmol) was added in two portions, 1 h interval. The reaction mixture was stirred an additional 3 h. The reaction was diluted with ethyl acetate and extracted with 0.1 N NaOH, then washed with water. The organic layer was separated and concentrated under reduced pressure to afford crude product that was purified by RP-HPLC (40-95% acetonitrile/water) to afford racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-benzoic acid methyl ester as an off-white solid (50.3 mg, 56.5%). HRMS (ES⁺) m/z Calcd for C₃₃H₃₃Cl₂F₂N₃O₃+H [(M+H)⁺]: 628.1940. found: 628.1939.

### Example 30

### Preparation of rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-propyl-pyrrolidine-2-carbonyl]-amino}-benzoic acid

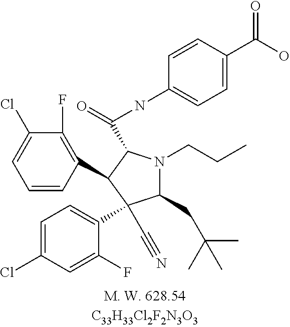

In a manner similar to the method described in Examples 1d, a mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester (75 mg, 0.125 mmol) and propionaldehyde (87 mg, 1.5 mmol) in AcOH (1.76 mL) was treated with sodium triacetoxyborohydride (Fluka, 176.0 mg, 0.83 mmol) followed by hydrolysis in a mixture of THF (3 mL) and methanol (1 mL), followed by 2N LiOH (1 mL). The reaction mixture was stirred at room temperature for 5 hours. The mixture was diluted with water and extracted with ethyl acetate (2×). The organic phase was separated, then concentrated under reduced pressure to afford the crude solid. RP-HPLC purification (40-95% acetonitrile/water) to afford racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-propyl-pyrrolidine-2-carbonyl]-amino}-benzoic acid as an off-white solid (18.3 mg, 23.3% over two steps). HRMS (ES⁺) m/z Calcd for C₃₃H₃₃Cl₂F₂N₃O₃+H [(M+H)⁺]: 628.1940. found: 628.1941.

### Example 31a

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-3-methoxy-benzoic acid methyl ester

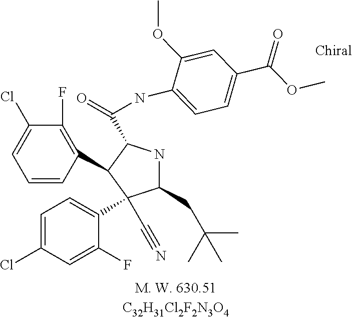

Reactants chiral (2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (300 mg, 0.642 mmol) and 2-(7-azabenzotriazol-1-yl)-n,n,n′,n′-tetramethyluronium hexafluorophosphate (HATU, 300 mg, 0.789 mmol) were added portionwise (3×, 30 min interval) to a solution of 4-amino-3-methoxy-benzoic acid methyl ester (0.310 g, 1.71 mmol) and diisopropylethyl amine (0.6 mL, 0.444 g, 3.4 mmol) in CH₂Cl₂ (20 mL) and was stirred at 25° C. for 14 h. The mixture was then diluted with CH₂Cl₂ and washed with water. The organic phase was separated, then concentrated under reduced pressure and purified by column chromatography (40 g Analgix, 0-100% ethyl acetate/heptane) to afford chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-3-methoxy-benzoic acid methyl ester (115.0 mg, 28.4%) as an off-white solid which was used in the next step without further purification. LCMS [(M+H)⁺]: calcd for C₃₂H₃₁Cl₂F₂N₃O₄: 629.17. found: 630.1.

### Example 31b

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid

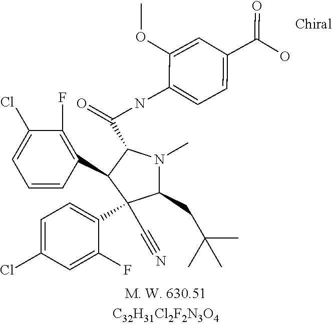

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-3-methoxy-benzoic acid methyl ester (26.0 mg, 0.0412 mmol) and paraformaldehyde (170 mg, 1.7 mmol) were combined with AcOH (4.00 mL to give a suspension. Sodium triacetoxyborohydride (Fluka, 100 mg, 0.472 mmol) was added in two portions 30 min apart. The reaction mixture was vigorously stirred for 14 h. The reaction mixture was diluted with 0.1 N NaOH and extracted with EtOAc. The organic layer was separated, filtered through a plug of MgSO₄/silica and concentrated in under reduced pressure to afford crude product chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl pyrrolidine-2-carbonyl]-amino}-3-methoxy-benzoic acid methyl ester (26 mg, 97.8%) that was taken directly to the hydrolysis in THF (3 mL) and methanol (1 mL), followed by 2N LiOH (1 mL). The reaction mixture was stirred at room temperature for 5 hours. The mixture was diluted with water and extracted with ethyl acetate (2×). The organic phase was separated, then concentrated under reduced pressure to afford the crude solid. RP-HPLC purification (40-95% acetonitrile/water) to afford chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid as an off-white solid (22.4 mg, 86.7%). HRMS (ES⁺) m/z Calcd for C₃₂H₃₁Cl₂F₂N₃O₄+H [(M+H)⁺]: 630.1733. found: 630.1735.

### Example 32

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-isobutyl-pyrrolidine-2-carbonyl]-amino}-benzoic acid

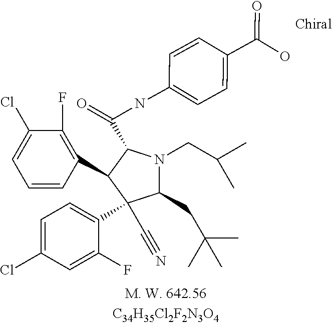

In a manner similar to the method described in Examples 1d, a mixture of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-benzoic acid methyl ester (75 mg, 0.125 mmol) and isobutraldehyde (45 mg, 0.624 mmol) in AcOH (3 mL) was treated with sodium triacetoxyborohydride (Fluka, 176.0 mg, 0.83 mmol) followed by hydrolysis in a mixture of THF (3 mL) and methanol (1 mL), followed by 2N LiOH (1 mL). The reaction mixture was stirred at room temperature for 5 hours. Then heated to 50° C. for 3 h. Cool mixture and dilute with water and extracted with ethyl acetate (2×). The organic phase was separated, then concentrated under reduced pressure to afford the crude solid. RP-HPLC purification (35-95% acetonitrile/water) to afford chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-isobutyl-pyrrolidine-2-carbonyl]-amino}-benzoic acid as an off-white solid (9.4 mg, 11.7% over two steps). HRMS (ES⁺) m/z Calcd for C₃₄H₃₅Cl₂F₂N₃O₃+H [(M+H)⁺]: 642.2097. found: 642.2097.

### Example 33

### Preparation of rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}benzoic acid

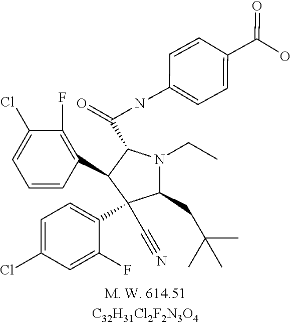

Racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-benzoic acid methyl ester (40 mg, 0.0636 mmol) was dissolved in a mixture of THF (3 mL) and methanol (1 mL), followed by 2N LiOH (1 mL). The reaction mixture was stirred at room temperature for 5 hours, then heated to 50° C. for 3 h. Cool mixture and dilute with water and extracted with ethyl acetate (2×). The organic phase was separated, then concentrated under reduced pressure to afford the crude solid. RP-HPLC purification (30-95% acetonitrile/water) to afford racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}benzoic acid as an off-white solid (12.1 mg, 30.9%). HRMS (ES⁺) m/z Calcd for C₃₄H₃₅Cl₂F₂N₃O₃+H [(M+H)⁺]: 614.1784. found: 614.1783.

### Example 34

### Preparation of rac-4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-benzoic acid

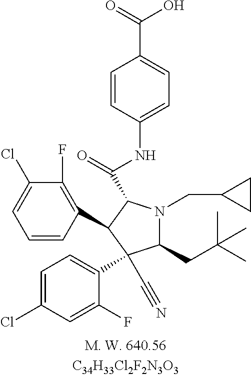

In a 10 mL round-bottomed flask, rac-methyl 4-((2R,3S,4R,5S)-3-(3-chloro-2-fluorophenyl)-4-(4-chloro-2-fluorophenyl)-4-cyano-5-neopentylpyrrolidine-2-carboxamido)benzoate (25.0 mg, 0.042 mmol) and cyclopropanecarbaldehyde (29.2 mg, 0.42 mmol) were combined with AcOH (1.00 mL) and CH₂Cl₂ (1 mL) to give a colorless solution. Sodium triacetoxyborohydride (Fluka, 176 mg, 0.83 mmol) was added in two portions 30 min apart. The reaction mixture was vigorously stirred for 5 h. The reaction mixture was quenched with 2.0 N NaOH and extracted with EtOAc (3×20 mL). The organic layers were dried over Na₂SO₄ and concentrated in vacuo. The crude material was used in the next step without further purification.

In a 10 mL round-bottomed flask, the crude methyl 4-((2R,3S,4R,5S)-3-(3-chloro-2-fluorophenyl)-4-(4-chloro-2-fluorophenyl)-4-cyano-1-(cyclopropylmethyl)-5-neopentylpyrrolidine-2-carboxamido)benzoate (30 mg, 0.046 mmol) was dissolved in a mixture of THF (1.8 mL)-MeOH (0.6 mL)-2N KOH (0.6 mL) to give a colorless solution. The reaction mixture was stirred at room temperature overnight. The reaction mixture was quenched with 2 N HCl and extracted with EtOAc (3×25 mL). The organic layers were combined, washed with H₂O (1×10 mL), sat NaCl (1×10 mL), and dried over Na₂SO₄ and concentrated in vacuo. The crude material was purified by preparative RP-HPLC to give rac-4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-benzoic acid as a white amorphous (21.3 mg, 72.6%). HRMS (ES⁺) m/z Calcd for C₃₄H₃₃Cl₂F₂N₃O₃+H [(M+H)⁺]: 640.1940. found: 640.1940.

### Example 35

### Preparation of rac-4-{[(2R,3S,4R,5S)-3-(3-chloro-2-fluoro-phenyl)-4-(4-chloro-2-fluoro-phenyl)-4-cyano-1-cyclobutylmethyl-5-(2,2-dimethyl-propyl)-pyrrolidine-2-carbonyl]-amino}-benzoic acid

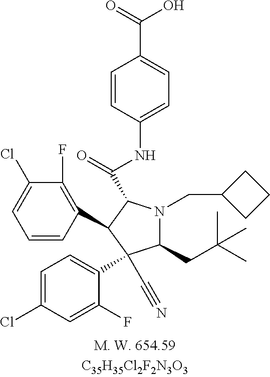

In a manner similar to the method described in Examples 1d, a mixture of rac-methyl 4-((2R,3S,4R,5S)-3-(3-chloro-2-fluorophenyl)-4-(4-chloro-2-fluorophenyl)-4-cyano-5-neopentylpyrrolidine-2-carboxamido)benzoate (30.0 mg, 0.05 mmol) and cyclobutanecarbaldehyde (42.0 mg, 0.50 mmol) in AcOH (1.00 mL) and CH₂Cl₂ (1 mL) was treated with sodium triacetoxyborohydride (Fluka, 212.0 mg, 1.0 mmol) followed by hydrolysis in a mixture of THF (1.8 mL)-MeOH (0.6 mL)-2N KOH (0.6 mL) to give, after purification by preparative RP-HPLC, rac-4-{[(2R,3S,4R,5S)-3-(3-chloro-2-fluoro-phenyl)-4-(4-chloro-2-fluoro-phenyl)-4-cyano-1-cyclobutylmethyl-5-(2,2-dimethyl-propyl)-pyrrolidine-2-carbonyl]-amino}-benzoic acid as a white amorphous 17.5 mg, 53.5% in 2 steps).

HRMS (ES⁺) m/z Calcd for C₃₅H₃₅Cl₂F₂N₃O₃+H [(M+H)⁺]: 654.2097. found: 654.2100.

### Example 36

### Preparation of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-benzoic acid

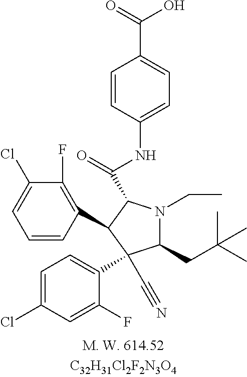

In a manner similar to the method described in Examples 1d, a mixture of rac-methyl 4-((2R,3S,4R,5S)-3-(3-chloro-2-fluorophenyl)-4-(4-chloro-2-fluorophenyl)-4-cyano-5-neopentylpyrrolidine-2-carboxamido)benzoate (30.0 mg, 50.0 μmol) and acetaldehyde (44.0 mg, 500 μmol) in AcOH (1.00 mL) and CH₂Cl₂ (1 mL) was treated with sodium triacetoxyborohydride (Fluka, 212.0 mg, 1.0 mmol) followed by hydrolysis in a mixture of THF (1.8 mL)-MeOH (0.6 mL)-2N KOH (0.6 mL) to give, after purification by preparative RP-HPLC, rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-benzoic acid as a white amorphous (21.5 mg, 70.5% in 2 steps).

HRMS (ES⁺) m/z Calcd for C₃₂H₃₁Cl₂F₂N₃O₃+H [(M+H)⁺]: 614.1784. found: 614.1784.

### Example 37

### Preparation of rac-4-{[(2R,3S,4R,5S)-3-(3-chloro-2-fluoro-phenyl)-4-(4-chloro-2-fluoro-phenyl)-4-cyano-1-(2-cyclohexyl-ethyl)-5-(2,2-dimethyl-propyl)-pyrrolidine-2-carbonyl]-amino}-benzoic acid

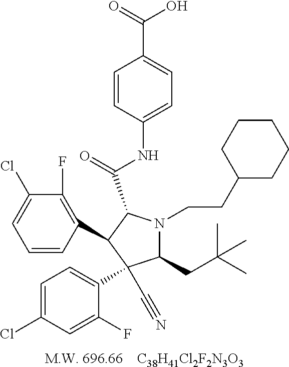

In a manner similar to the method described in Examples 1d, a mixture of rac-methyl 4-((2R,3S,4R,5S)-3-(3-chloro-2-fluorophenyl)-4-(4-chloro-2-fluorophenyl)-4-cyano-5-neopentylpyrrolidine-2-carboxamido)benzoate (30.0 mg, 500 μmol) and cyclohexyl-acetaldehyde (63.0 mg, 500 μmol) in AcOH (1.00 mL) and CH₂Cl₂ (1 mL) was treated with sodium triacetoxyborohydride (Fluka, 212.0 mg, 1.0 mmol) followed by hydrolysis in a mixture of THF (1.8 mL)-MeOH (0.6 mL)-2N KOH (0.6 mL) to give, after purification by preparative RP-HPLC, rac-4-{[(2R,3S,4R,5S)-3-(3-chloro-2-fluoro-phenyl)-4-(4-chloro-2-fluoro-phenyl)-4-cyano-1-(2-cyclohexyl-ethyl)-5-(2,2-dimethyl-propyl)-pyrrolidine-2-carbonyl]-amino}-benzoic acid as a white amorphous (5.9 mg, 17.0% in 2 steps).

HRMS (ES⁺) m/z Calcd for C₃₈H₄₁Cl₂F₂N₃O₃+H [(M+H)⁺]: 694.2410. found: 694.2409.

### Example 38a

### Preparation of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid methyl ester

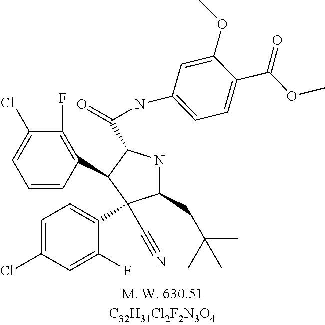

Reactants rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (1.0 g, 2.14 mmol) and 2-(7-azabenzotriazol-1-yl)-n,n,n′,n′-tetramethyluronium hexafluorophosphate (HATU, 895 mg, 2.35 mmol) were added portionwise (3×, 30 min interval) to a solution of 4-amino-2-methoxy-benzoic acid methyl ester (1.85 g, 10.2 mmol) and diisopropylethyl amine (0.561 mL, 0.415 g, 3.21 mmol) in CH₂Cl₂ (30 mL) and was stirred at 25° C. for 14 h. The mixture was then diluted with CH₂Cl₂ and washed with water. The organic phase was separated, then concentrated under reduced pressure and purified by column chromatography (40 g Analgix, 0-30% ethyl acetate/heptane, then ramp to 100% ethyl acetate) to afford rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid methyl ester (1.24 g, 91.9%) as an off-white solid which was used in the next step without further purification. LCMS [(M+H)⁺]: calcd for C₃₂H₃₁Cl₂F₂N₃O₄: 629.17. found: 630.0.

### Example 38b

### Preparation of chiral 4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-2-methoxy-benzoic acid

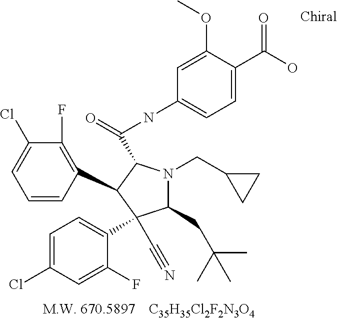

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid methyl ester (75.0 mg, 0.119 mmol) and cyclopropanecarboxaldehyde (0.1 mL, 93 mg, 1.33 mmol) were combined with AcOH (3.00 mL) to give a colorless solution. Sodium triacetoxyborohydride (Fluka, 176 mg, 0.83 mmol) was added in two portions, 30 min apart. The reaction mixture was vigorously stirred for 3 h. The reaction mixture was diluted with 0.1 N NaOH and extracted with EtOAc. The organic layer was separated and concentrated under reduced pressure to afford crude product chiral 4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-2-methoxy-benzoic acid methyl ester mix that was carried directly to the next step.

The crude product chiral 4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-2-methoxy-benzoic acid methyl ester mix (40 mg, 0.058.4 mmol) was dissolved in THF (3 mL) and methanol (1 mL), followed by 2N LiOH (1 mL). The reaction mixture was stirred at room temperature for 12 hours. The reaction mixture was diluted with water and extracted with ethyl acetate (2×). The organic phase was separated, then concentrated under reduced pressure to afford the crude solid. RP-HPLC purification (40-95% acetonitrile/water) to afford chiral 4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-2-methoxy-benzoic acid as an off-white solid (17 mg, 43.3%). HRMS (ES⁺) m/z Calcd for C₃₅H₃₅Cl₂F₂N₃O₄+H [(M+H)⁺]: 670.2046. found: 670.2046.

### Example 39a

### Preparation of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino-3-methoxy-benzoic acid methyl ester

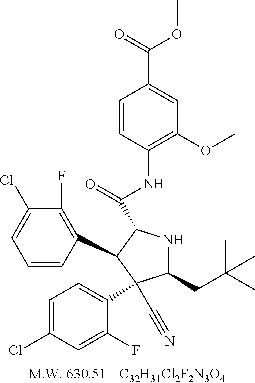

In a 10 mL round-bottomed flask, rac-(2R,3S,4R,5S)-3-(3-chloro-2-fluorophenyl)-4-(4-chloro-2-fluorophenyl)-4-cyano-5-neopentylpyrrolidine-2-carboxamide (233.2 mg, 500 μmol), DIPEA (Aldrich, 517 mg, 699 μl, 4.00 mmol) and diphenylphosphinic chloride (Aldrich, 473 mg, 2.00 mmol) were combined with CH₂Cl₂ (2.00 mL) to give a light brown solution. The reaction mixture was stirred at rt for 5 min. and methyl 4-amino-3-methoxybenzoate (90.6 mg, 500 μmol) was added. The reaction mixture was stirred at rt overnight to give a yellowish suspension. The reaction mixture was quenched with H₂O and extracted with CH₂Cl₂ (3×25 mL). The organic layers were dried over Na₂SO₄ and concentrated in vacuo. The crude material was purified by flash chromatography (silica gel, 12 g, 1% to 25% EtOAc in hexanes) to give rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino-3-methoxy-benzoic acid methyl ester (214.0 mg, 76.4%).

### Example 39b

### Preparation of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid

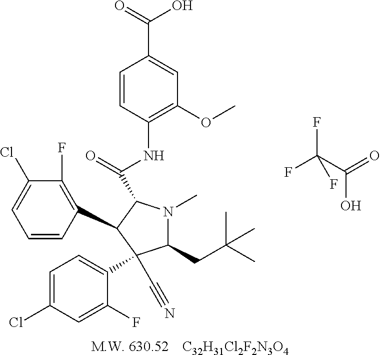

In a manner similar to the method described in Examples 1d, a mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino-3-methoxy-benzoic acid methyl ester (31.5 mg, 50.0 μmol) and paraformaldehyde (30.0 mg, 1.00 mmol) in AcOH (1.00 mL) and DCM (1 mL) was treated with sodium triacetoxyborohydride (Fluka, 212.0 mg, 1.0 mmol) followed by hydrolysis in a mixture of THF (1.8 mL)-MeOH (0.6 mL)-2N KOH (0.6 mL) to give, after purification by preparative RP-HPLC, rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid trifluoroacitic acid salt as a white amorphous (16.5 mg, 44.4% in 2 steps). HRMS (ES⁺) m/z Calcd for C₃₂H₃₁Cl₂F₂N₃O₄+H [(M+H)⁺]: 630.1733. found: 630.1737.

### Example 40

### Preparation of rac-4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-3-methoxy-benzoic acid

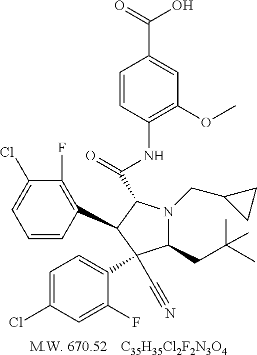

In a manner similar to the method described in Examples 1d, a mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino-3-methoxy-benzoic acid methyl ester (31.5 mg, 50.0 μmol) and cyclopropanecarbaldehyde (Aldrich, 35.0 mg, 0.50 mmol) in AcOH (1.00 mL) and DCM (1 mL) was treated with sodium triacetoxyborohydride (Fluka, 212.0 mg, 1.0 mmol) followed by hydrolysis in a mixture of THF (1.8 mL)-MeOH (0.6 mL)-2N KOH (0.6 mL) to give, after purification by preparative RP-HPLC, rac-4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-3-methoxy-benzoic acid as a white amorphous (8.8 mg, 26.3% in 2 steps). HRMS (ES⁺) m/z Calcd for C₃₅H₃₅Cl₂F₂N₃O₄+H [(M+H)⁺]: 670.2046. found: 670.2047.

### Example 41

### Preparation of racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-2-methoxy-benzoic acid

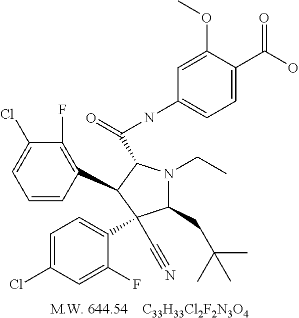

In a manner similar to the method described in Examples 1d, a mixture of racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid methyl ester (75 mg, 0.142 mmol) and acetaldehyde (0.1 mL, 78 mg, 1.77 mmol) in AcOH (3 mL) was treated with sodium triacetoxyborohydride (Fluka, 200.0 mg, 0.83 mmol) followed by hydrolysis in a mixture of THF (3 mL) and methanol (1 mL), followed by 2N LiOH (1 mL). The reaction mixture was stirred at room temperature for 14 hours. Dilute with water and extracted with ethyl acetate (2×). The organic phase was separated, then concentrated under reduced pressure to afford the crude solid. RP-HPLC purification (40-95% acetonitrile/water) to afford racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-2-methoxy-benzoic acid as an off-white solid (44.1 mg, 75.1%). HRMS (ES⁺) m/z Calcd for C₃₃H₃₃Cl₂F₂N₃O₄+H [(M+H)⁺]: 644.1889. found: 644.1889.

### Example 42

### Preparation of racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-methoxy-benzoic acid

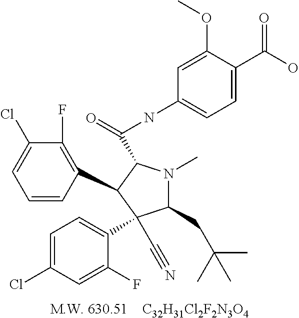

In a round-bottomed flask, rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid methyl ester (75.0 mg, 0.119 mmol) and paraformaldehyde (30 mg, 0.999 mmol) were combined with AcOH (3.00 mL) and CH₂Cl₂ (1 mL) to give a colorless suspension. Sodium triacetoxyborohydride (Fluka, 176 mg, 0.83 mmol) was added in two portions 30 min apart. The reaction mixture was vigorously stirred for 14 h. The reaction mixture was diluted with 0.1 N NaOH and extracted with EtOAc. The organic layer was separated, filtered through celite and concentrated in under reduced pressure to afford crude product racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-methoxy-benzoic acid methyl ester (52 mg, 67.8%).

In a round-bottomed flask, the crude racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-methoxy-benzoic acid methyl (50 mg, 0.0776 mmol) was dissolved in THF (3 mL) and methanol (1 mL), followed by 2N LiOH (1 mL). The reaction mixture was stirred at room temperature for 14 hours. The reaction mixture was diluted with water and extracted with ethyl acetate (2×). The organic phase was separated, then concentrated under reduced pressure to afford the crude solid. RP-HPLC purification (40-95% acetonitrile/water) to afford 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-2-methoxy-benzoic acid as an off-white solid (18.6 mg, 38%). HRMS (ES⁺) m/z Calcd for C₃₂H₃₁Cl₂F₂N₃O₄+H [(M+H)⁺]: 630.1733. found: 630.1733.

### Example 43

### Preparation of racemic 4-{[(2R,3S,4R,5S)-1-butyl-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid

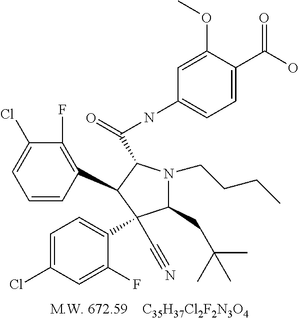

In a manner similar to the method described in Examples 1d, a mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid methyl ester (75 mg, 0.142 mmol) and butanal (0.1 mL, 80 mg, 1.11 mmol) in AcOH (3 mL) was treated with sodium triacetoxyborohydride (Fluka, 176.0 mg, 0.83 mmol) followed by hydrolysis in a mixture of THF (6 mL) and methanol (2 mL), followed by 2N LiOH (2 mL). The reaction mixture was stirred at room temperature for 14 hours. Dilute with water and extracted with ethyl acetate (2×). The organic phase was separated, then concentrated under reduced pressure to afford the crude solid. RP-HPLC purification (40-95% acetonitrile/water) to afford racemic 4-{[(2R,3S,4R,5S)-1-butyl-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid as an off-white solid (48.5 mg, 30.3% over two steps). HRMS (ES⁺) m/z Calcd for C₃₅H₃₇Cl₂F₂N₃O₄+H [(M+H)⁺]: 672.2202. found: 672.2202.

### Example 44

### Preparation of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-3-methoxy-benzoic acid

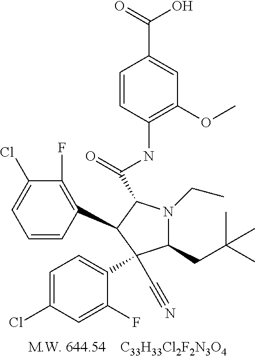

In a manner similar to the method described in Examples 1d, a mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino-3-methoxy-benzoic acid methyl ester (63.1 mg, 0.1 mmol) and acetaldehyde (Aldrich, 44.1 mg, 1.00 mmol) in AcOH (1.00 mL) and DCM (1 mL) was treated with sodium triacetoxyborohydride (Fluka, 414.0 mg, 2.00 mmol) followed by hydrolysis in a mixture of THF (1.8 mL)-MeOH (0.6 mL)-2N KOH (0.6 mL) to give, after purification by preparative RP-HPLC, rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-3-methoxy-benzoic acid as a white amorphous (26.5 mg, 41.1% in 2 steps).

HRMS (ES⁺) m/z Calcd for C₃₃H₃₃Cl₂F₂N₃O₄+H [(M+H)⁺]: 644.1889. found: 644.1891.

### Example 45

### Preparation of racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(3-methyl-butyl)-pyrrolidine-2-carbonyl]-amino}-2-methoxy-benzoic acid

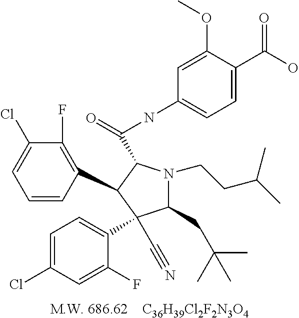

In a manner similar to the method described in Examples 1d, a mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid methyl ester (75 mg, 0.142 mmol) and isovaleraldehyde (0.1 mL, 80.3 mg, 0.932 mmol) in AcOH (3 mL) was treated with sodium triacetoxyborohydride (Fluka, 176.0 mg, 0.83 mmol) followed by hydrolysis in a mixture of THF (3 mL) and methanol (1 mL), followed by 2N LiOH (1 mL). The reaction mixture was stirred at room temperature for 14 hours. Dilute with water and extracted with ethyl acetate (2×). The organic phase was separated, then concentrated under reduced pressure to afford the crude solid. RP-HPLC purification (40-95% acetonitrile/water) to afford racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(3-methyl-butyl)-pyrrolidine-2-carbonyl]-amino}-2-methoxy-benzoic acid as an off-white solid (33 mg, 33.8% for two steps). HRMS (ES⁺) m/z Calcd for C₃₆H₃₉Cl₂F₂N₃O₄+H [(M+H)⁺]: 686.2359. found: 686.2361.

### Example 46

### Preparation of racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-pentyl-pyrrolidine-2-carbonyl]-amino}-2-methoxy-benzoic acid

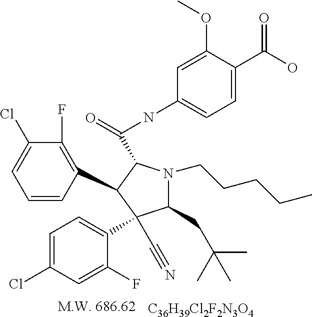

In a manner similar to the method described in Examples 2e, a mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid methyl ester (250 mg, 0.397 mmol) and valeraldehyde (0.1 mL, 80.3 mg, 0.932 mmol) in AcOH (3 mL) was treated with sodium triacetoxyborohydride (Fluka, 176.0 mg, 0.83 mmol) followed by hydrolysis in a mixture of THF (3 mL) and methanol (1 mL), followed by 2N LiOH (1 mL). The reaction mixture was stirred at room temperature for 14 hours. Dilute with water and extracted with ethyl acetate (2×). The organic phase was separated, then concentrated under reduced pressure to afford the crude solid. RP-HPLC purification (40-95% acetonitrile/water) to afford racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-pentyl-pyrrolidine-2-carbonyl]-amino}-2-methoxy-benzoic acid as an off-white solid (28.5 mg, 29.2% for two steps). HRMS (ES⁺) m/z Calcd for C₃₆H₃₉Cl₂F₂N₃O₄+H [(M+H)⁺]: 686.2359. found: 686.2363.

### Example 47

### Preparation of racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-pent-4-enyl-pyrrolidine-2-carbonyl]-amino}-2-methoxy-benzoic acid

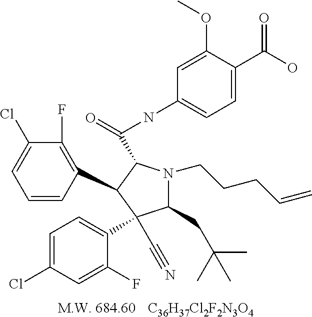

In a manner similar to the method described in Examples 1d, a mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid methyl ester (110 mg, 0.174 mmol) and 4-pentenal (0.1 mL, 85.2 mg, 0.932 mmol) in AcOH (3 mL) was treated with sodium triacetoxyborohydride (Fluka, 176.0 mg, 0.83 mmol) followed by hydrolysis in a mixture of THF (3 mL) and methanol (1 mL), followed by 2N LiOH (1 mL). The reaction mixture was stirred at room temperature for 14 hours. Dilute with water and extracted with ethyl acetate (2×). The organic phase was separated, then concentrated under reduced pressure to afford the crude solid. RP-HPLC purification (40-95% acetonitrile/water) to afford 4-{[(2R,3S,4R,5S)-3-(3-chloro-2-fluoro-phenyl)-4-(4-chloro-2-fluoro-phenyl)-4-cyano-5-(2,2-dimethyl-propyl)-1-pent-4-enyl-pyrrolidine-2-carbonyl]-amino}-2-methoxy-benzoic acid as an off-white solid (28.5 mg, 23.9% for two steps). HRMS (ES⁺) m/z Calcd for C₃₆H₃₇Cl₂F₂N₃O₄+H [(M+H)⁺]: 684.2202. found: 684.2201.

### Example 48

### Preparation of racemic 4-{[(2R,3S,4R,5S)-3-(3-chloro-2-fluoro-phenyl)-4-(4-chloro-2-fluoro-phenyl)-4-cyano-1-(2-cyclohexyl-ethyl)-5-(2,2-dimethyl-propyl)-pyrrolidine-2-carbonyl]-amino}-2-methoxy-benzoic acid trifluoroacetate salt

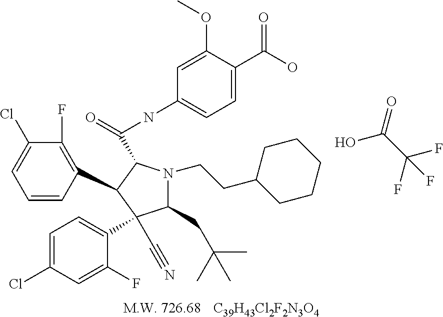

In a manner similar to the method described in Examples 1d, a mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid methyl ester (84.5 mg, 0.134 mmol) and 2-cyclohexylacetaldehyde (0.1 mL, 0.792 mmol) in AcOH (3 mL) and dichloromethane (1 mL) was treated with sodium triacetoxyborohydride (Fluka, 240.0 mg, 1.13 mmol) [followed reaction by LCMS (C8 column, 70-100% acetonitrile/water)] followed by hydrolysis in a mixture of THF (3 mL) and methanol (1 mL), followed by 2N LiOH (1 mL). The reaction mixture was stirred at room temperature for 14 hours. Dilute with water and extracted with ethyl acetate (2×). The organic phase was separated, then concentrated under reduced pressure to afford the racemic 4-{[(2R,3S,4R,5S)-3-(3-chloro-2-fluoro-phenyl)-4-(4-chloro-2-fluoro-phenyl)-4-cyano-1-(2-cyclohexyl-ethyl)-5-(2,2-dimethyl-propyl)-pyrrolidine-2-carbonyl]-amino}-2-methoxy-benzoic acid trifluoroacetate salt as an off-white solid (4.9 mg, 5% over two steps). HRMS (ES⁺) m/z Calcd for C₃₉H₄₃Cl₂F₂N₃O₄+H [(M+H)⁺]: 726.2672. found: 726.2672.

### Example 49

### Preparation of racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-propyl-pyrrolidine-2-carbonyl]-amino}-2-methoxy-benzoic acid

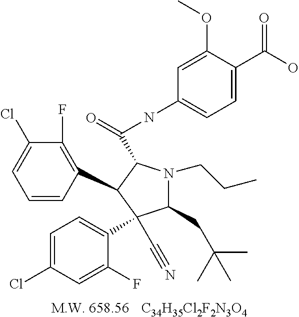

In a manner similar to the method described in Examples 1d, a mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid methyl ester (100 mg, 0.159 mmol) and propionaldehyde (0.1 mL, 1.39 mmol) in AcOH (3 mL) was treated with sodium triacetoxyborohydride (Fluka, 176.0 mg, 0.83 mmol) followed by hydrolysis in a mixture of THF (3 mL) and methanol (1 mL), followed by 2N LiOH (1 mL). The reaction mixture was stirred at room temperature for 4 hours. Dilute with water and extracted with ethyl acetate (2×). The organic phase was separated, then concentrated under reduced pressure to afford the crude solid. RP-HPLC purification (40-95% acetonitrile/water) to afford racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-propyl-pyrrolidine-2-carbonyl]-amino}-2-methoxy-benzoic acid as an off-white solid (44 mg, 42.1%). HRMS (ES⁺) m/z Calcd for C₃₄H₃₅Cl₂F₂N₃O₄+H [(M+H)⁺]: 658.2046. found: 658.2046.

### Example 50

### Preparation of rac-(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (4-carbamoyl-2-methoxy-phenyl)-amide

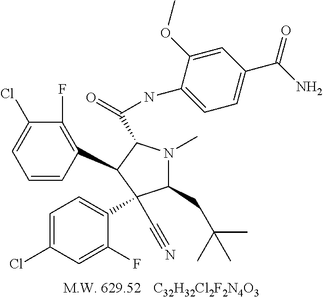

A mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid (100 mg, 0.159 mmol), 2-(7-azabenzotriazol-1-yl)-n,n,n′,n′-tetramethyluronium hexafluorophosphate (HATU, 90.5 mg, 0.238 mmol) and ammonia in 1,4-dioxane (0.5 M, 3.17 mL, 1.59 mmol) in CH₂Cl₂ (10 mL) was stirred at rt 3 h. LCMS of reaction mixture, showed reaction partially complete, additional ammonia in 1,4 dioxane (0.5 M, 3 mL, 1.5 mmol) was added to the reaction mixture and stirred overnight at RT. The mixture was then diluted with CH₂Cl₂ and washed with water, brine. The organic phase was separated, then concentrated under reduced pressure and purified by reverse phase chromatography (35-95% of ACN/water) to afford rac-(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (4-carbamoyl-2-methoxy-phenyl)-amide (12.8 mg, 12.8%) as an white solid. HRMS (ES⁺) m/z Calcd for C₃₂H₃₂Cl₂F₂N₄O₃+H [(M+H)⁺]: 629.1893. found: 629.1894.

### Example 51

### Preparation of rac-(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (2-methoxy-4-methylcarbamoyl-phenyl)-amide

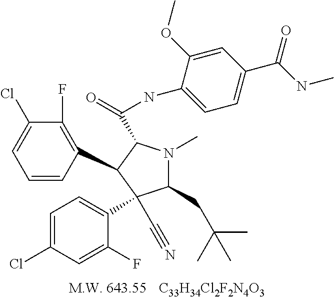

A mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid (50 mg, 0.0793 mmol), 2-(7-azabenzotriazol-1-yl)-n,n,n′,n′-tetramethyluronium hexafluorophosphate (HATU, 45.2 mg, 0.119 mmol) and methyl amine in THF (2 M, 0.4 mL, 0.8 mmol) in CH₂Cl₂ (10 mL) was stirred at rt 3 h. LCMS of reaction mixture, showed reaction partially complete, additional methyl amine in THF (2 M, 0.4 mL, 0.8 mmol) was added to the reaction mixture and stirred overnight at RT. The mixture was then diluted with CH₂Cl₂ and washed with water, brine. The organic phase was separated, then concentrated under reduced pressure and purified by reverse phase chromatography (35-95% of ACN/water) to afford rac-(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (2-methoxy-4-methylcarbamoyl-phenyl)-amide (16.1 mg, 31.5%) as an off-white solid. HRMS (ES⁺) m/z Calcd for C₃₃H₃₄Cl₂F₂N₄O₃+H [(M+H)⁺]: 643.2049. found: 643.2049.

### Example 52a

### Preparation of 1-(3-amino-pyrazol-1-yl)-2-methyl-propan-2-ol

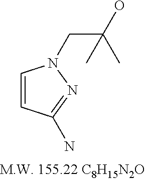

A mixture of 2-methyl-1-(3-nitro-pyrazol-1-yl)-propan-2-ol (0.3 g, 1.6 mmol), Zn dust (0.42 g, 6.5 mmol), ammonium chloride (0.85 g, 16 mmol) and methanol (2 mL) was microwaved at 120° C. for 10 min. The resulting suspension was filtered through celite with methanol and THF washes. Solvent was removed under reduced pressure to yield crude product that was triterated with ethyl acetate and filtered again to get rid of ammonium chloride salt. Product 1-(3-amino-pyrazol-1-yl)-2-methyl-propan-2-ol was a yellow solid (233 mg, 94%).

### Example 52b

### Preparation of 2-methyl-1-(3-nitro-pyrazol-1-yl)-propan-2-ol

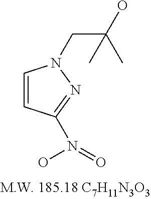

A mixture of 3-nitro-1H-pyrazole (10.0 g, 88.43 mmol), 2,2-dimethyl-oxirane (15.7 mL, 176.9 mmol), potassium carbonate (18.2 g, 132 mmol) and DMF (100 mL) was stirred at 100° C. for 1 h, then stirred overnight at rt. The mixture was then diluted with ethyl acetate and water, the organic layer was separated, dried over Na₂SO₄, and filtered. The resulting mixture was concentrated under reduced pressure to yield the crude product that was purified (50% EtOAc/heptane) to yield the product 2-methyl-1-(3-nitro-pyrazol-1-yl)-propan-2-ol as a waxy solid (4.88 g, 30%).

### Example 52c

### Preparation of 1-[2-methyl-2-((R)-1-oxiranylmethoxy)-propyl]-3-nitro-1H-pyrazole

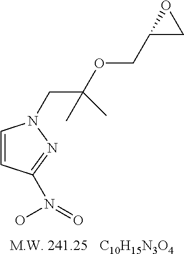

A mixture of 2-methyl-1-(3-nitro-pyrazol-1-yl)-propan-2-ol (1.31 g, 7.07 mmol), and DMF (60 mL) was stirred at 0° C. for 5 min, then NaH (60% dispersion in oil, 0.85 g, 21.2 mmol) was added and stirred 20 min at 0° C. R-(−)-glycidyl-3-nitrobenzenesulfonate (2.75 g, 10.6 mmol) was added and stirred at 0° C. for 1 h then warmed to rt for 14 h. The mixture was then diluted with NH₄Cl (s), ethyl acetate, the organic phase was separated, washed with NaHCO₃ (satd) dried with Na₂SO₄, and filtered. The mixture was then concentrated and purified by column chromatography (40-240 g Analogix column, 70% EtoAc/heptane to yield the product 1-[2-methyl-2-((R)-1-oxiranylmethoxy)-propyl]-3-nitro-1H-pyrazole as a white solid (0.7 g, 41%).

### Example 52d

### Preparation of 1-[2-methyl-2-((R)-1-oxiranylmethoxy)-propyl]-1H-pyrazol-3yl amine

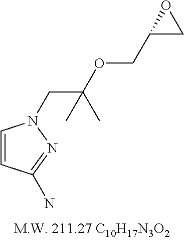

A mixture of 1-[2-methyl-2-((R)-1-oxiranylmethoxy)-propyl]-3-nitro-1H-pyrazole (0.3 g, 1.24 mmol), and ethyl acetate (15 mL), ethanol (15 mL) was subjected to the H-Cube (Thales Nano) at 1 mL/min at 10° C., 10 psi hydrogen. The first time through not completely reduced. Resubjected and complete reduction of nitro group by NMR. Solvent was removed under reduced pressure to afford 1-[2-methyl-2-((R)-1-oxiranylmethoxy)-propyl]-1H-pyrazol-3-ylamine as an oil (0.27 g, 100%).

### Example 52e

### Preparation of rac-(2R,3S,4R,5S)-3-(3-chloro-2-fluoro-phenyl)-4-(4-chloro-2-fluoro-phenyl)-4-cyano-5-(2,2-dimethylpropyl)-pyrrolidine-2-carboxylic acid {1-[2-methyl-2-((R)-1-oxiranylmethoxy)-propyl]-1H-pyrazol-3-yl}-amide

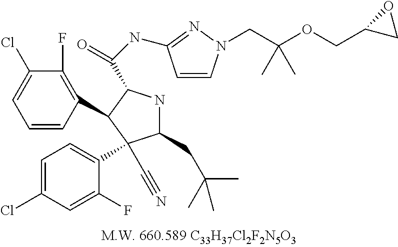

A mixture of rac-(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (0.56 g, 1.2 mmol), 1-[2-methyl-2-((R)-1-oxiranylmethoxy)-propyl]-1H-pyrazol-3-ylamine (0.27 g, 1.12 mmol), 2-(7-azabenzotriazol-1-yl)-n,n,n′,n′-tetramethyluronium hexafluorophosphate (HATU, 0.912 g, 2.4 mmol) and iPr₂NEt (1.5 mL, 8.4 mmol) in CH₂Cl₂ (50 mL) was stirred at rt overnight. The mixture was then diluted with CH₂Cl₂ and washed with water, brine. The organic phase was separated, filtered and dried over Na₂SO₄. The mixture was then concentrated and purified by column chromatography (25-80 g Analgix column, 1-100% ethyl acetate/heptane) to give rac-(2R,3S,4R,5S)-3-(3-chloro-2-fluoro-phenyl)-4-(4-chloro-2-fluoro-phenyl)-4-cyano-5-(2,2-dimethylpropyl)-pyrrolidine-2-carboxylic acid {1-[2-methyl-2-((R)-1-oxiranylmethoxy)-propyl]-1H-pyrazol-3-yl}-amide (222 mg, 28%) as an off-white solid. HRMS (ES⁺) m/z Calcd for C₃₃H₃₇Cl₂F₂N₅O₃+H [(M+H)⁺]: 660.2315. found: 660.2312.

### Example 52f

### Preparation of rac-(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carboxylic acid {1-[2-((R)-3-dimethylamino-2-hydroxy-propoxy)-2-methyl-propyl]-1H-pyrazol-3-yl}-amide

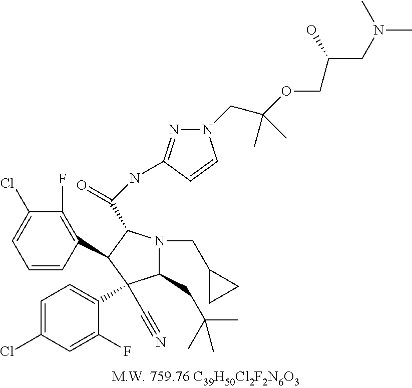

A mixture of rac-(2R,3S,4R,5S)-3-(3-chloro-2-fluoro-phenyl)-4-(4-chloro-2-fluoro-phenyl)-4-cyano-5-(2,2-dimethylpropyl)-pyrrolidine-2-carboxylic acid {1-[2-methyl-2-((R)-1-oxiranylmethoxy)-propyl]-1H-pyrazol-3-yl}-amide (100 mg, 0.151 mmol), acetic acid (0.1 mL) and cyclopropanecarboxaldehyde (Acros organics, 63.7 mg, 0.908 mmol) were dissolved in dichloroethane (1 mL) then sodium triacetoxyborohydride (96.3 mg, 0.454 mmol) was added and stirred at 25° C. for 1 h. Incomplete by LCMS, additional sodium triacetoxyborohydride (0.1 g, 0.455 mmol) was added and stirred 4 h at 25° C. Monitored reaction by LCMS (1:1, SM:PDT) also showed epoxide starting to open, work up with water an ethyl acetate. Carried crude mix to next step (epoxide ring opening).

A mixture of rac-(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carboxylic acid {1-[2-methyl-2-((R)-1-oxiranylmethoxy)-propyl]-1H-pyrazol-3-yl}-amide mix (108 mg, 0.151 mmol) and dimethyl amine were dissolved with 2-propanol (1 mL) and THF (1 mL) and subjected to microwaved conditions for 10.5 min at 130° C. Reaction worked up by concentration under reduced pressure and purified by reverse phase chromatography (20-95% of ACN/water) to afford rac-(2R,3S,4R,5S)-3-(3-chloro-2-fluoro-phenyl)-4-(4-chloro-2-fluorophenyl)-4-cyano-1-cyclopropylmethyl-5-(2,2-dimethyl-propyl)-pyrrolidine-2-carboxylic acid {1-[2-((R)-3-dimethylamino-2-hydroxy-propoxy)-2-methyl-propyl]-1H-pyrazol-3-yl}-amide (26 mg, 22.7%) as an off-white solid. HRMS (ES⁺) m/z Calcd for C₃₉H₅₀Cl₂F₂N₆O₃+H [(M+H)⁺]: 759.3363. found: 759.3367.

### Example 53

### Preparation of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-(5-hydroxy-pentyl)-pyrrolidine-2-carbonyl]-amino}-benzoic acid

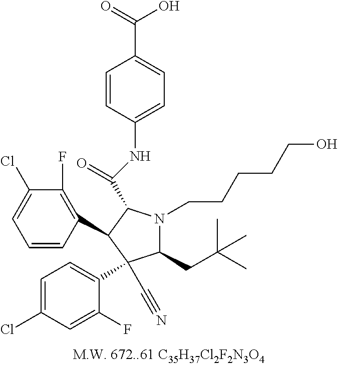

In a manner similar to the method described in Examples 1d, a mixture of rac-methyl 4-((2R,3S,4R,5S)-3-(3-chloro-2-fluorophenyl)-4-(4-chloro-2-fluorophenyl)-4-cyano-5-neopentylpyrrolidine-2-carboxamido)benzoate (30.0 mg, 0.05 mmol) and 5-hydroxypentanal (Aldrich, 51.0 mg, 0.50 mmol) in AcOH (2.00 mL) and CH₂Cl₂ (2 mL) was treated with sodium triacetoxyborohydride (Fluka, 212.0 mg, 1.0 mmol) followed by hydrolysis in a mixture of THF (1.8 mL)-MeOH (0.6 mL)-2N KOH (0.6 mL) to give, after purification by preparative RP-HPLC, rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-(5-hydroxy-pentyl)-pyrrolidine-2-carbonyl]-amino}-benzoic acid as a white amorphous (7.3 mg, 18.6% in 2 steps).

HRMS (ES⁺) m/z Calcd for C₃₅H₃₇Cl₂F₂N₃O₄+H [(M+H)⁺]: 672.2202. found: 672.2204.

### Example 54

### Preparation of chiral 5-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2H-[1,2,4]triazole-3-carboxylic acid methyl ester

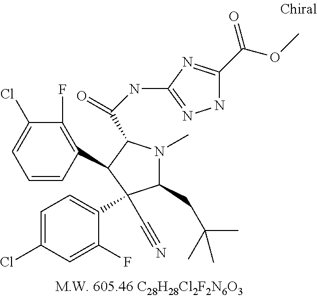

Reactant methyl 3-amino-1H-1,2,4-triazole-5-carboxylate (88.6 mg, 623 μmol) was dissolved in CH₂Cl₂ (12.5 mL) was stirred at 25° C., then iPr₂NEt (0.1 mL, 74 mg, 573 μmol) was added followed by addition of chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (50.0 mg, 104 μmol) and 2-(7-azabenzotriazol-1-yl)-n,n,n′,n′-tetramethyluronium hexafluorophosphate (HATU, 59.2 mg, 156 μmol) portionwise and stirred 14 h at rt. The mixture was diluted with CH₂Cl₂ and washed with water. The organic phase was separated, and concentrated under reduced pressure and purified by RP-HPLC (40-95% acetonitrile/water) to afford chiral 5-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2H-[1,2,4]triazole-3-carboxylic acid methyl ester as an off-white solid (12 mg, 19.1%). HRMS (ES⁺) m/z Calcd for C₂₈H₂₈Cl₂F₂N₆O₃+H [(M+H)⁺]: 605.1641. found 605.1639

### Example 55

### Preparation of chiral 2-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3H-imidazole-4-carboxylic acid trifluoracetate salt

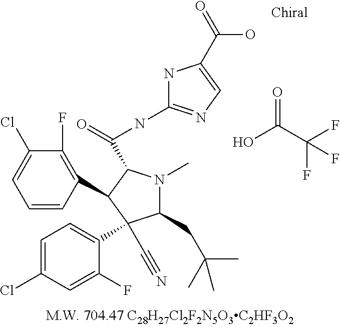

In a flask chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (50.0 mg, 104 mmol) and CDI (40.4 mg, 249 μmol) were dissolved in dichloromethane (2.14 mL) to give a colorless solution. The reaction mixture was stirred at 25° C. for 0.5 h, then ethyl 2-amino-1H-imidazole-5-carboxylate (64.5 mg, 0.415 μmol) was added and stirred overnight at rt. Checked by LCMS only 50% complete; worked up, by diluting with CH₂Cl₂ and water, the organic layer was separated and concentrated under reduced pressure to afford the crude material that was taken directly to the hydrolysis step.

The chiral 2-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3H-imidazole-4-carboxylic acid ethyl ester mix (129 mg, 208 mmol) was dissolved in ethanol (5 mL) followed by 2M KOH (2 mL). The reaction mixture was stirred at room temperature for 15 hours. The reaction mixture was concentrated under reduced pressure to afford the crude solid. The mixture was dissolved in DMSO for purification RP-HPLC purification (40-85% acetonitrile/water) upon concentration of fractions noticed compound readily hydrolyzes to scaffold acid with imidazole as LG. Repurified RP-HPLC (40-85% acetonitrile/water) with lyophilizing pure fractions to afford white lyophilized solid chiral 2-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3H-imidazole-4-carboxylic acid trifluoracetate salt as a white lyophilized solid (8 mg, 5.46%). HRMS (ES⁺) m/z Calcd for C₂₈H₂₇Cl₂F₂N₅O₃+H [(M+H)⁺]: 590.1532 found: 590.1534.

### Example 56

### Preparation of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-(5-hydroxy-pentyl)-pyrrolidine-2-carbonyl]-amino}-3-methoxy-benzoic acid

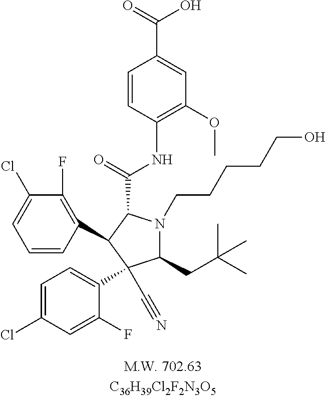

In a manner similar to the method described in Examples 1d, a mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino-3-methoxy-benzoic acid methyl ester (31.5 mg, 0.05 mmol) and 5-hydroxypentanal (Aldrich, 51.0 mg, 0.50 mmol) in AcOH (2.00 mL) and CH₂Cl₂ (2 mL) was treated with sodium triacetoxyborohydride (Fluka, 212.0 mg, 1.0 mmol) followed by hydrolysis in a mixture of THF (1.8 mL)-MeOH (0.6 mL)-2N KOH (0.6 mL) to give, after purification by preparative RP-HPLC, (7.3 mg, 18.6% in 2 steps).

HRMS (ES⁺) m/z Calcd for C₃₆H₃₉Cl₂F₂N₃O₅+H [(M+H)⁺]: 702.2308. found: 702.2304.

### Example 57a

### Preparation of chiral (2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid ethylamide

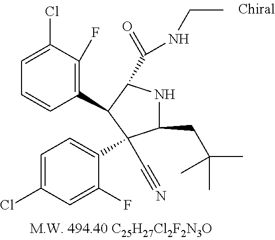

Reactant ethylamine in methanol (12 mL, 24 mmol, 2 M) was dissolved in CH₂Cl₂ (12.5 mL) was stirred at 25° C., followed by addition of chiral (2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid (400.0 mg, 856 mmol) and 2-(7-azabenzotriazol-1-yl)-n,n,n′,n′-tetramethyluronium hexafluorophosphate (HATU, 488 mg, 1.28 mol) portionwise and then stirred overnight at rt. The reaction not complete, methanol may be degrading HATU? The mixture was diluted with CH₂Cl₂ and washed with water. The organic phase was separated, and concentrated under reduced pressure and purified by RP-HPLC (30-95% acetonitrile/water) to afford chiral (2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid ethylamide as an off-white foam (0.3 g, 70.9%). HRMS (ES⁺) m/z Calcd for C₂₅H₂₇Cl₂F₂N₃O+H [(M+H)⁺]: 494.1572 found 494.1574

### Example 57b

### Preparation of (2R,3S,4R,5S)-4-(4-chloro-2-fluoro-phenyl)-3-(3-chloro-2-fluoro-phenyl)-4-cyano-1-cyclopropylmethyl-5-(2,2-dimethyl-propyl)-pyrrolidine-2-carboxylic acid ethylamide

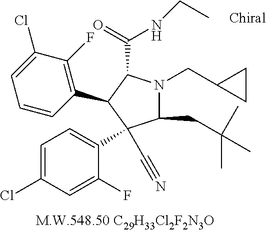

In a manner similar to the method described in Examples 1d, a mixture of (2R,3S,4R,5S)-3-(3-chloro-2-fluorophenyl)-4-(4-chloro-2-fluorophenyl)-4-cyano-N-ethyl-5-neopentylpyrrolidine-2-carboxamide (49.5 mg, 0.1 mmol) and cyclopropanecarboxaldehyde (Acros organics, 140.0 mg, 2.0 mmol) in AcOH (1.0 mL) and CH₂Cl₂ (1.0 mL) was treated with sodium triacetoxyborohydride (Fluka, 424.0 mg, 2.0 mmol) and the reaction mixture was quenched with 2.0 N NaOH and extracted with EtOAc (3×20 mL). The organic layers were dried over Na2SO4 and concentrated in vacuo. The crude material was purified by flash chromatography (silica gel, 4 g, 1% to 20% EtOAc in hexanes) to give (2R,3S,4R,5S)-4-(4-chloro-2-fluoro-phenyl)-3-(3-chloro-2-fluoro-phenyl)-4-cyano-1-cyclopropylmethyl-5-(2,2-dimethyl-propyl)-pyrrolidine-2-carboxylic acid ethylamide (48.6 mg, 88.5% in 2 steps). HRMS (ES⁺) m/z Calcd for C₂₉H₃₃Cl₂F₂N₃O+H [(M+H)⁺]: 548.2042. found: 548.2042.

### Example 58a

### Preparation of chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid

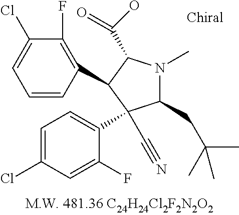

In a round-bottomed flask, chiral (2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid methyl ester (300 mg, 623 mmol) was dissolved in acetic acid (2 mL) and cooled to 0° C., then paraformaldehyde (200 mg, 6.66 mmol) and sodium triacetoxyborohydride (Fluka, 396 mg, 1.87 mmol) were added and stirred at 10° C. The reaction mixture was vigorously stirred for 16 h at rt. The reaction was monitored by LCMS, more sodium triacetoxyborohydride (0.396 mg, 1.87 mmol) and paraformaldehyde (200 mg, 6.66 mmol) with stirring at rt, until complete by LCMS. The reaction mixture was diluted with water and extracted with EtOAc. The mixture was filtered through glass filter membrane (GF/F) then the organic was separated then concentrated under reduced pressure to afford the crude product that was purified with silica chromatography (12 g Analogix, 0-100% Ethyl acetate/heptane) to afford chiral (2R,3S,4R,5S)-3-(3-chloro-2-fluoro-phenyl)-4-(4-chloro-2-fluoro-phenyl)-4-cyano-5-(2,2-dimethyl-propyl)-1-methylpyrrolidine-2-carboxylic acid methyl ester (230 mg, 74.5%). The product was taken directly to the hydrolysis step.

In a round-bottomed flask, the chiral (2R,3S,4R,5S)-3-(3-chloro-2-fluoro-phenyl)-4-(4-chloro-2-fluoro-phenyl)-4-cyano-5-(2,2-dimethyl-propyl)-1-methylpyrrolidine-2-carboxylic acid methyl ester (220 mg, 0.0776 mmol) was dissolved in THF (6 mL) and methanol (2 mL), followed by 2N LiOH (2 mL). The reaction mixture was stirred at room temperature for 5 hours. The reaction mixture was diluted with water and extracted with ethyl acetate (2×). The organic phase was separated, then concentrated under reduced pressure to afford the crude solid. RP-HPLC purification (40-95% acetonitrile/water) to afford chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid as an off-white solid (199.6 mg, 93.4%). HRMS (ES⁺) m/z Calcd for C₂₄H₂₄Cl₂F₂N₂O₂+H [(M+H)⁺]: 481.1256. found: 481.1255.

### Example 58b

### Preparation of chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid [1-(2-hydroxy-2-methyl-propyl)-1H-pyrazol-3-yl]-amide

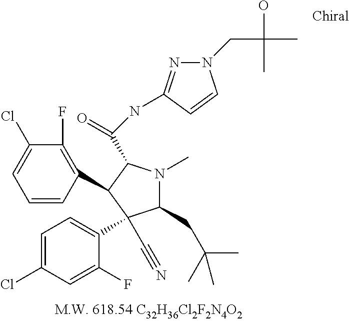

Reactant 1-(3-amino-pyrrol-1-yl)-2-methyl-propan-2-ol (96.7 mg, 0.623 μmol) was dissolved in CH₂Cl₂ (12.5 mL) was stirred at 25° C. then iPr₂NEt (27.2 μL, 4 μmol) was added followed by addition of chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (50.0 mg, 104 mmol) and 2-(7-azabenzotriazol-1-yl)-n,n,n′,n′-tetramethyluronium hexafluorophosphate (HATU, 59.2 mg, 156 mmol) portionwise and stirred 14 h at rt. The mixture was diluted with CH₂Cl₂ and washed with water. The organic phase was separated, and concentrated under reduced pressure and purified by RP-HPLC (40-95% acetonitrile/water) to afford chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid [1-(2-hydroxy-2-methyl-propyl)-1H-pyrazol-3-yl]-amide as an off-white solid (12 mg, 18.7%). HRMS (ES⁺) m/z Calcd for C₃₂H₃₆Cl₂F₂N₄O₂+H [(M+H)⁺]: 618.2209. found: 618.2210.

### Example 59a

### Preparation of (R)-Amino-cyclopropyl-acetic acid methyl ester

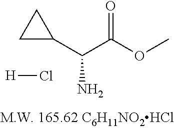

A mixture of BOC-D-cyclopropylglycine (Chem-Impex, Inc., 0.25 g, 1.16 mmol), and methanol (4 mL) was stirred at 25° C., thionyl chloride (0.1 mL, 1.39 mmol) was CAREFULLY added dropwise (cool to 0° C. if larger scale) and stirred an additional 30 minutes until complete by tlc. The mixture was concentrated under reduced pressure to afford a white crystalline solid (180 mg, 93.6%).

### Example 59b

### Preparation of (R)-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-cyclopropyl-acetic acid methyl ester

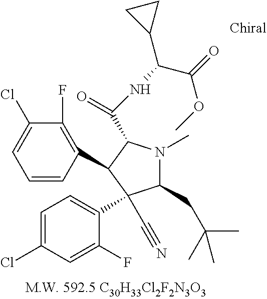

Reactant chiral R-methyl-2-amino-2-cyclopropylacetate hydrochloride (168 mg, 1.01 mmol) was dissolved in CH₂Cl₂ (25 mL) with iPr₂NEt (0.181 mL, 1.04 mmol) was stirred at 25° C., followed by addition of chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (100.0 mg, 0.208 mmol) and 2-(7-azabenzotriazol-1-yl)-n,n,n′,n′-tetramethyluronium hexafluorophosphate (HATU, 118 mg, 0.312 mmol) portionwise and then stirred 14 h at rt. The mixture was diluted with CH₂Cl₂ and washed with water. The organic phase was separated and concentrated under reduced pressure to afford the crude material. Purification by column chromatography (8 g Anologix column, 1-100% Ethyl acetate/heptane) to afford (R)-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-cyclopropyl-acetic acid methyl ester as a waxy off-white solid (101 mg, 82.1%) which was used in the next step without further purification (Example 60).

### Example 60

### Preparation of (R)-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-cyclopropyl-acetic acid

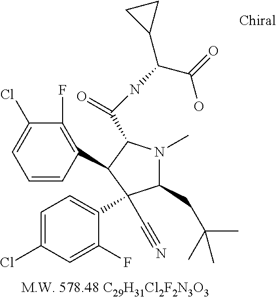

In a round-bottomed flask, the (R)-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-cyclopropyl-acetic acid methyl ester (Example 59b, 101 mg, 0.101 mmol) was dissolved in THF (6 mL) and methanol (2 mL), followed by 2N LiOH (2 mL). The reaction mixture was stirred at room temperature for 3 hours. The reaction mixture was diluted with water and extracted with ethyl acetate (2×). The organic phase was separated, then concentrated under reduced pressure to afford the product (R)-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-cyclopropyl-acetic acid as an off-white solid (50.3 mg, 85.9%). HRMS (ES⁺) m/z Calcd for C₂₉H₃₁Cl₂F₂N₃O₃+H [(M+H)⁺]: 578.1784. found: 578.1779.

### Example 61

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(2-ethoxycarbonyl-cyclopropylmethyl)-pyrrolidine-2-carbonyl]-amino}-3-methoxy-benzoic acid methyl ester

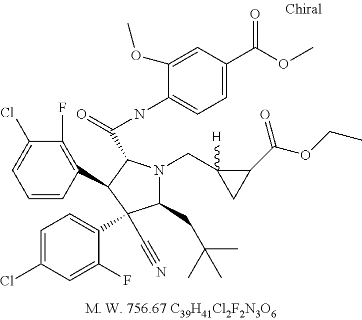

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-3-methoxy-benzoic acid methyl ester (100.0 mg, 0.159 mmol) and ethyl 2-formyl-1-cyclopropanecarboxyalate (0.1 mL, 107 mg, 0.756 mmol, predominately trans) were combined with AcOH (1.2 mL) to give a colorless solution. Sodium triacetoxyborohydride (Fluka, 300 mg, 1.42 mmol) was added in two portions, 30 min apart. The reaction mixture was vigorously stirred for 3 h. The reaction mixture was diluted with 0.1 N NaOH and extracted with EtOAc. The organic layer was separated and concentrated under reduced pressure to afford crude product that was purified via RP-HPLC (50-95% acetonitrile/water) to afford chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(2-ethoxycarbonyl-cyclopropylmethyl)-pyrrolidine-2-carbonyl]-amino}-3-methoxy-benzoic acid methyl ester as an off-white solid (6 mg, 5%). HRMS (ES⁺) m/z Calcd for C₃₉H₄₁Cl₂F₂N₃O₆+H [(M+H)⁺]: Calcd 756.2413. found 756.2413.

### Example 62

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-prop-2-ynyl-pyrrolidine-2-carbonyl]-amino}-3-methoxy-benzoic acid

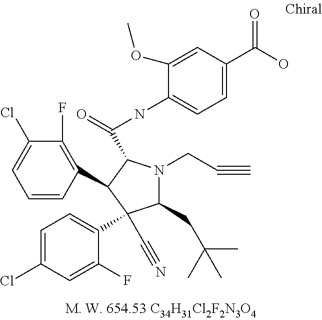

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-3-methoxy-benzoic acid methyl ester (50.0 mg, 79.3 μmol) and 3-trimethylsilylpropynal (0.1 mL, 0.792 mmol) were combined with AcOH (0.60 mL) and stirred at rt. Sodium triacetoxyborohydride (Fluka, 150 mg, 0.708 mmol) was added in two portions, 30 min apart and vigorously stirred for 3 h. The reaction mixture was diluted with 0.1 N NaOH and extracted with EtOAc. The organic layer was separated and concentrated under reduced pressure to afford crude product chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(3-trimethylsilanyl-prop-2-ynyl)-pyrrolidine-2-carbonyl]-amino}-3-methoxy-benzoic acid (52 mg, 88.5%) that was carried directly to the next step.

Compound 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(3-trimethylsilanyl-prop-2-ynyl)-pyrrolidine-2-carbonyl]-amino}-3-methoxy-benzoic acid (50 mg, 0.0675 mmol) was dissolved in THF (1.5 mL) and methanol (0.5 mL), followed by 2N LiOH (0.5 mL) addition. The reaction mixture was stirred at room temperature for 14 hours. The reaction mixture was diluted with water and extracted with ethyl acetate (2×). The organic phase was separated, then concentrated under reduced pressure to afford chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-prop-2-ynyl-pyrrolidine-2-carbonyl]-amino}-3-methoxy-benzoic acid as an off-white solid (41 mg, 92.8%). HRMS (ES⁺) m/z Calcd for C₃₄H₃₁Cl₂F₂N₃O₄+H [(M+H)⁺]: 654.1733 found: 654.1730.

### Example 63

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(ethoxycarbonylmethyl-carbamoyl)-pyrrolidine-2-carbonyl]-amino}-3-methoxy-benzoic acid

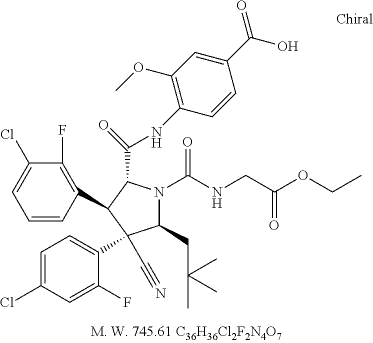

To a lo mL microwave vial was added chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-3-methoxy-benzoic acid (31.0 mg, 0.050 mmol) and ethyl 2-isocyanatoacetate (6.49 mg, 0.050 mmol) in CH₂Cl₂ (3 ml) to give a suspension. The vial was capped and heated in the microwave at 120° C. for 15 min. The reaction mixture was poured into water (5 mL) and extracted with EtOA (3×20 mL). The organic layer was separated and concentrated under reduced pressure to afford crude product which was purified by RP-HPLC to give chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(ethoxycarbonylmethyl-carbamoyl)-pyrrolidine-2-carbonyl]-amino}-3-methoxy-benzoic acid (15.3 mg, 40.8%) as white powder.

HRMS (ES⁺) m/z Calcd for C₃₆H₃₆Cl₂F₂N₄O₇+H [(M+H)⁺]: calc. found. LCMS calc=745.61 found: 745.2.

### Example 64

### Preparation of chiral (2S,3R,4S,5R)-4-(3-chloro-2-fluoro-phenyl)-3-(4-chloro-2-fluoro-phenyl)-2-(2,2-dimethyl-propyl)-5-((R)-2-hydroxymethyl-pyrrolidine-1-carbonyl)-1-methyl-pyrrolidine-3-carbonitrile

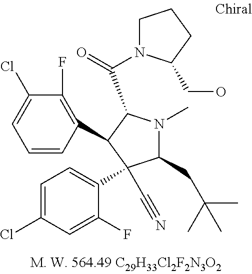

Reactant chiral R-pyrrolidin-2-ylmethanol (105 mg, 623 mmol) was dissolved in CH₂Cl₂ (25 mL) was stirred at 25° C., followed by addition of chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (100.0 mg, 208 mmol) and 2-(7-azabenzotriazol-1-yl)-n,n,n′,n′-tetramethyluronium hexafluorophosphate (HATU, 118 mg, 312 μmol) portionwise and then stirred 14 h at rt. The mixture was then diluted with CH₂Cl₂ and washed with water. The organic phase was separated, and concentrated under reduced pressure. Purification by RP-HPLC (40-85% acetonitrile/water) to afford chiral (2S,3R,4S,5R)-4-(3-chloro-2-fluoro-phenyl)-3-(4-chloro-2-fluoro-phenyl)-2-(2,2-dimethyl-propyl)-5-((R)-2-hydroxymethyl-pyrrolidine-1-carbonyl)-1-methyl-pyrrolidine-3-carbonitrile as an off-white solid (67.5 mg, 57.6%). HRMS (ES⁺) m/z Calcd for C₂₉H₃₃Cl₂F₂N₃O₂+H [(M+H)⁺]: 564.1991. found: 564.1987.

### Example 65

### Preparation of chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid [(S)-2-hydroxy-1-(1H-imidazol-4-ylmethyl)-ethyl]-amide trifluoroacetate salt

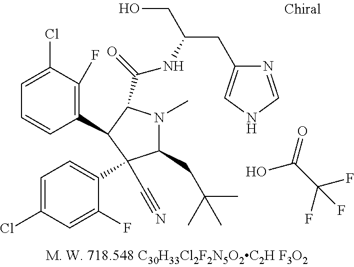

Reactant (S)-2-amino-3-(1H-imidaxol-4-yl)propan-1-ol dihydrochloride (133 mg, 623 μmol) was dissolved in CH₂Cl₂ (25 mL) was stirred at 25° C., then iPr₂NEt (0.218 mL, 161 mg, 1.25 mmol) was added followed by addition of chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (100.0 mg, 208 μmol) and 2-(7-azabenzotriazol-1-yl)-n,n,n′,n′-tetramethyluronium hexafluorophosphate (HATU, 118 mg, 312 μmol) portionwise and then stirred 14 h at rt. The mixture was then diluted with CH₂Cl₂ and washed with water. The organic phase was separated, and concentrated under reduced pressure and purified by RP-HPLC (10-65% acetonitrile/water) to afford chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid [(S)-2-hydroxy-1-(1H-imidazol-4-ylmethyl)-ethyl]-amide trifluoroacetate salt as an off-white solid (42 mg, 28.1%). HRMS (ES⁺) m/z Calcd for C₃₀H₃₃Cl₂F₂N₅O₂+H [(M+H)⁺]: 604.2052. found: 604.2050.

### Example 66

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-1-ethyl-1H-pyrrole-2-carboxylic acid

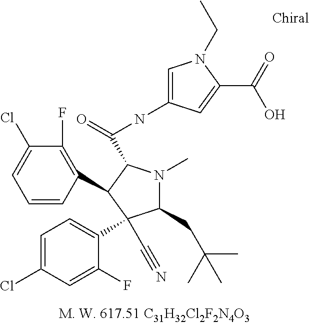

In a round-bottomed flask, methyl 4-amino-1-ethyl-1H-pyrrole-2-carboxylate (349 mg, 0.792 mmol) was combined with CH₂Cl₂ (25 mL) with iPr₂NEt (80.5 mg, 0.109 mL, 0.623 mmol) and stirred at 25° C., followed by addition of chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (200.0 mg, 415 mmol) and 2-(7-azabenzotriazol-1-yl)-n,n,n′,n′-tetramethyluronium hexafluorophosphate (HATU, 237 mg, 623 mmol) in two portions. The reaction mixture was stirred 14 h at rt. The mixture was diluted with CH₂Cl₂ and washed with water. The organic phase was separated and concentrated under reduced pressure to afford the crude material. Purification by column chromatography (1-100% Ethyl Acetate/heptane) to afford product chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-1-ethyl-1H-pyrrole-2-carboxylic acid methyl ester (140 mg, 53.4%) that was carried directly to the next step.

Compound chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-1-ethyl-1H-pyrrole-2-carboxylic acid methyl ester (125 mg, 0.198 mmol) was dissolved in THF (9 mL) and methanol (3 mL), followed by 2N LiOH (3 mL) addition. The reaction mixture was stirred at room temperature for 4 hours. The reaction mixture was diluted with water and extracted with ethyl acetate (2×). The organic phase was separated, then concentrated under reduced pressure to afford chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-1-ethyl-1H-pyrrole-2-carboxylic acid as a white powder (25.2 mg, 20.6%). HRMS (ES⁺) m/z Calcd for C₃₁H₃₂Cl₂F₂N₄O₃+H [(M+H)⁺]: 617.1893. Found: 617.1890.

### Example 67 & 68

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1S,2S)-2-methoxycarbonyl-cyclopropylmethyl)-pyrrolidine-2-carbonyl]-amino}-3-methoxy-benzoic acid methyl ester and chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1R,2R)-2-methoxycarbonyl-cyclopropylmethyl)-pyrrolidine-2-carbonyl]-amino}-3-methoxy-benzoic acid methyl ester

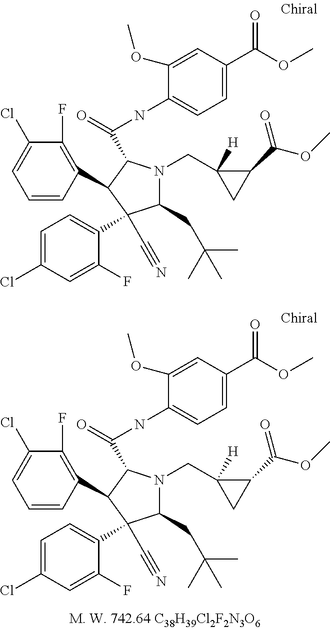

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(2-ethoxycarbonyl-cyclopropylmethyl)-pyrrolidine-2-carbonyl]-amino}-3-methoxy-benzoic acid methyl ester (180 mg, 0.238 mmol) was dissolved in THF (10 mL) and methanol (5 mL), followed by 2N LiOH (5 mL) addition. The reaction mixture was stirred at room temperature for 4 hours, transesterification complete. The reaction mixture was concentrated under reduced pressure and purified with RP-HPLC (50-95% acetonitrile/water) to afford chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1S,2S)-2-methoxycarbonyl-cyclopropylmethyl)-pyrrolidine-2-carbonyl]-amino}-3-methoxy-benzoic acid methyl ester as a white powder (8 mg, 4.53%). HRMS (ES⁺) m/z Calcd for C₃₈H₃₉Cl₂F₂N₃O₆+H [(M+H)⁺]: 742.2257. Found: 742.2258, and chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1R,2R)-2-methoxycarbonyl-cyclopropylmethyl)-pyrrolidine-2-carbonyl]-amino}-3-methoxy-benzoic acid methyl ester as a white powder (7 mg, 3.96%). HRMS (ES⁺) m/z Calcd for C₃₈H₃₉Cl₂F₂N₃O₆+H [(M+H)⁺]: 742.2257. Found: 742.2256.

### Example 69 & 70

### Preparation of chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1S,2S)-2-ethoxycarbonyl-cyclopropylmethyl)-pyrrolidine-2-carbonyl]-amino}-3-methoxy-benzoic acid and chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1R,2R)-2-ethoxycarbonyl-cyclopropylmethyl)-pyrrolidine-2-carbonyl]-amino}-3-methoxy-benzoic acid

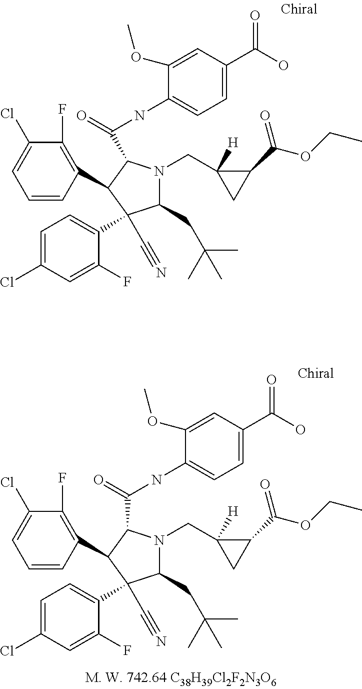

In a round-bottomed flask, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-3-methoxy-benzoic acid methyl ester (500.0 mg, 0.793 mmol) was combined with AcOH (13.3 mL) then sodium triacetoxyborohydride (Fluka, 500 mg, 2.36 mmol) was added and stirred 15 min. Make sure flask is cool to touch then add ethyl 2-formyl-1-cyclopropanecarboxyalate (1.0 mL, 1.07 g, 7.56 mmol, predominately trans). The reaction mixture was vigorously stirred overnight at room temperature. The reaction mixture was diluted with 0.1 N NaOH and extracted with EtOAc. The organic layer was separated and concentrated under reduced pressure to afford crude products that was taken directly to next step by dissolving in THF (30 mL) followed by addition of 2N LiOH (15 mL) addition. The reaction mixture was stirred at room temperature for 24 hours, then heated to 50° C. for 48 h. Careful work up by separation of aqueous layer, wash with brine, then organic layer separated and concentrated under reduced pressure and purified with RP-HPLC (50-95% acetonitrile/water, 0.05% TFA using JSPHERE ODS-H80, 100×30 mm, s=4 micron; YMCJH08S04-1030WT) to afford chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1S,2S)-2-ethoxycarbonyl-cyclopropylmethyl)-pyrrolidine-2-carbonyl]-amino}-3-methoxy-benzoic acid as a white powder (12 mg, 4.08%). HRMS (ES⁺) m/z Calcd for C₃₈H₃₉Cl₂F₂N₃O₆+H [(M+H)⁺]: 742.2257. Found: 742.2253, and chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1R,2R)-2-ethoxycarbonyl-cyclopropylmethyl)-pyrrolidine-2-carbonyl]-amino}-3-methoxy-benzoic acid as a white powder (10 mg, 3.4%). HRMS (ES⁺) m/z Calcd for C₃₈H₃₉Cl₂F₂N₃O₆+H [(M+H)⁺]: 742.2257. Found: 742.2254.

### Example 71

### Preparation of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-(5-hydroxy-pentyl)-pyrrolidine-2-carbonyl]-amino}-2-methoxy-benzoic acid

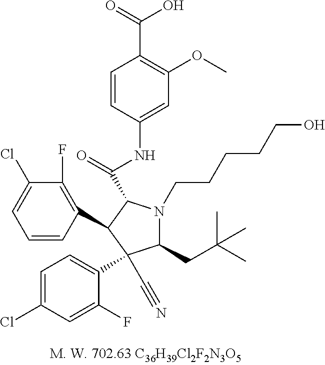

In a manner similar to the method described in Examples 1d, a mixture of rac-4-{[(2R,3S,4R,5S)-3-(3-chloro-2-fluoro-phenyl)-4-(4-chloro-2-fluoro-phenyl)-4-cyano-5-(2,2-dimethyl-propyl)-pyrrolidine-2-carbonyl]-amino-2-methoxy-benzoic acid methyl ester (63.0 mg, 0.10 mmol) and 5-hydroxypentanal (Aldrich, 102.0 mg, 1.00 mmol) in AcOH (2.00 mL) and CH₂Cl₂ (2 mL) was treated with sodium triacetoxyborohydride (Fluka, 414.0 mg, 2.0 mmol) followed by hydrolysis in a mixture of THF (1.8 mL)-MeOH (0.6 mL)-2N KOH (0.6 mL) to give, after purification by preparative RP-HPLC, (33.6 mg, 47.8% in 2 steps).

HRMS (ES⁺) m/z Calcd for C₃₆H₃₉Cl₂F₂N₃O₅+H [(M+H)⁺]: 702.2308. found: 702.2306.

### Example 72

The pharmacological properties of the compounds of this invention may be confirmed by a number of pharmacological assays. The exemplified antiproliferative activity assay which follows has been carried out with the compounds according to the invention. The compounds of the invention inhibited cancer cell proliferation with IC₅₀ values of less than 20 μM.

Cell proliferation was evaluated by the tetrazolium dye assay according to the procedure of Denizot and Lang (Denizot, F. and Lang, R. (1986) Rapid colorimetric assay for cell growth and survival. Modification to the terrazolium dye procedure giving improved sensitivity and reliability. J. Immunol. Methods 89, 271-277). The cell line used was SJSA, an osteocarcinoma cell line obtained from the American Type Cell Culture Collection (ATCC; Rockville, Md.). The cells were grown in PRMI with 10% FBS (Fetal Bovine Serum, Invitrogen).

Cells were plated at the appropriate seeding density to give logarithmic growth over the course of the assay in a 96-well tissue culture plate. Plates were incubated overnight at 37° C. in a humidified incubator with 5% CO₂. The next day, test compounds were serially diluted 1:3 in the appropriate medium containing 3% DMSO. One-tenth final volume of each dilution was added in duplicate to the plates containing cells. The same volume of 3% DMSO in medium was added to a row of control wells. Thus, the final concentration of DMSO in all wells was 0.3%. The plates were returned to the incubator, and at set time points (determined by their growth curves) plates were analyzed as follows: 3-(4,5-dimethylthiazole-2-yl)-2,5-diphenyl-2H-tetrazolium bromide (thiazolyl blue; MTT; Sigma) was added to each well to yield a final concentration of 1 mg/ml. Plates were returned to the incubator for 2.5 hours. The MTT-containing medium was removed and the resulting formazan metabolite was solubilized in 100% ethanol with shaking for 15 minutes at room temperature. Absorbances were read in a microtiter plate reader at a wavelength of 570 nm with a 650 nm reference. Percent inhibition (% INH) was calculated by subtracting the blank from all wells, then subtracting the ratio of the average absorbance of each triplicate (S_{AVE}) by the average of the controls (C_{AVE}) from 1.00. The final product was then multiplied by 100 (% INH=(1.00−S_{AVE}/C_{AVE})×100). The concentration at which 50% inhibition (IC₅₀) of cell proliferation is obtained was determined from the linear regression of a plot of the logarithm of the concentration versus percent inhibition. The IC₅₀ values are shown in Table 1 below.

TABLE I
This table shows the IC₅₀s of representative compounds of the instant
invention in the cell-based assay (“MTT”) assay

| Example Number | TT IC₅₀(μM) |
|---|---|
| Example 25 | 0.39 |
| Example 26 | 0.32 |
| Example 27 | 1.95 |
| Example 31 | 0.19 |
| Example 32 | 0.66 |
| Example 33 | 1.31 |
| Example 34 | 0.30 |
| Example 37 | 0.11 |
| Example 38 | 0.09 |
| Example 39 | 0.04 |
| Example 40 | 0.13 |
| Example 41 | 1.23 |
| Example 42 | 1.36 |
| Example 43 | 1.14 |
| Example 44 | 0.22 |
| Example 45 | 0.42 |
| Example 47 | 0.62 |
| Example 48 | 0.43 |
| Example 49 | 1.49 |
| Example 50 | 0.37 |
| Example 51 | 0.46 |
| Example 54 | 0.75 |
| Example 55 | 4.00 |
| Example 56 | 0.03 |
| Example 58 | 3.59 |
| Example 61 | 0.68 |
| Example 62 | 0.08 |

What is claimed:

1. A compound of formula I 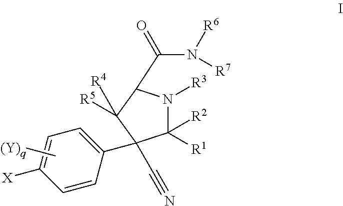
wherein
X is selected from the group consisting of H, F, Cl, Br, I, cyano, nitro, lower alkynyl, cycloalkyl, lower alkyl, lower alkenyl and lower alkoxy;
Each Y is independently selected from the group consisting of H, F, Cl, Br, I, CN, OH, nitro, lower alkyl, cycloalkyl, lower alkoxy, lower alkenyl, cycloalkenyl, lower alkynyl, aryl, heteroaryl, heterocycle, COOR′, OCOR′, CONR′R″, NR′COR″, NR″SO₂R′, SO₂NR′R″ and NR′R″, wherein R′ and R″ are independently selected from
H,
lower alkyl optionally substituted with halogen, hydroxy, NH₂, NH-lower alkyl, N(lower-alkyl)₂, oxo, carboxy, carboxy lower alkyl, cycloalkyl and heteroaryl,
lower cycloalkyl optionally substituted with halogen, lower alkoxy, lower alkyl, carboxy, carboxy lower alkoxy, carboxy lower alkyl, oxo, CN, and NHSO₂-lower alkyl,
lower alkenyl optionally substituted with halogen, hydroxy, NH₂ and NH-lower alkyl and N(lower-alkyl)₂,
lower alkynyl optionally substituted with halogen, hydroxy, NH₂ and NH-lower alkyl and N(lower-alkyl)₂,
lower cycloalkenyl optionally substituted with halogen, lower alkoxy, lower alkyl, carboxy, carboxy lower alkoxy, oxo and CN,
aryl optionally substituted with halogen, lower alkoxy, hydroxy, lower alkyl, lower alkenyl, cycloalkyl, carboxy, carboxy lower alkoxy, carboxy lower alkyl, oxo, R¹ and CN, CONR¹R²
hetereoaryl optionally substituted with halogen, lower alkoxy, lower alkyl, carbonyl, carboxy, carboxy lower alkyl, carboxy lower alkoxy, oxo, R¹ and CN, and
hetereocycle optionally substituted with halogen, lower alkoxy, carboxy, carboxy lower alkoxy, oxo CN, R¹, SO₂-lower alkyl and lower alkyl that is optionally substituted with oxo,
or alternatively, when Y is CONR′R″ or SO₂NR′R″, R′ and R″ together with the N to which they are attached can form a ring having 3 to 7 atoms, said ring being selected from or cycloalkyl, cycloalkenyl heteroaryl and or heterocycle, said ring optionally being substituted with halogen, lower alkoxy, lower alkyl, carboxy, carboxy lower alkoxy, oxo and CN;
one of R¹ and R² is H and the other is selected from the group consisting of
lower alkyl optionally substituted with halogen, hydroxy, NH₂, NH-lower alkyl and N(lower-alkyl)₂,
lower alkenyl optionally substituted with halogen, hydroxy, NH₂, NH-lower alkyl and N(lower-alkyl)₂,
aryl optionally substituted with halogen, lower alkoxy, lower alkyl, carboxy, carboxy lower alkoxy, oxo and CN,
heteroaryl optionally substituted with halogen, lower alkoxy, lower alkyl, carboxy, carboxy lower alkoxy, oxo and CN,
heterocycle optionally substituted with R′, halogen, lower alkoxy, carboxy, carboxy lower alkoxy, oxo, CN, SO₂—R′,
cycloalkyl and cycloalkyl substituted with halogen, lower alkoxy, lower alkyl, hydroxycarbony, carboxy, carboxy lower alkoxy, oxo and CN, and
cycloalkenyl and cycloalkenyl substituted with halogen, lower alkoxy, lower alkyl, hydroxycarbony, carboxy, carboxy lower alkoxy, oxo and CN;
R³ is selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, wherein R′ and R″ are as defined above for Y,
or alternatively, R³ together with R¹ or R² can form a ring having 3 to 7 atoms, said ring being selected from heteroaryl and heterocycle, said ring optionally being substituted with halogen, lower alkoxy, lower alkyl, hydroxycarbony, carboxy, carboxy lower alkoxy, oxo and CN;
one of R⁴ and R⁵ is selected from the group consisting of hydrogen, lower alkyl and CF₃ and the other is selected from the group consisting of
lower alkyl optionally substituted with halogen, hydroxy, NH₂, NH-lower alkyl and N(lower-alkyl)₂,
lower alkenyl optionally substituted with halogen, hydroxy, NH₂, NH-lower alkyl and N(lower-alkyl)₂,
aryl optionally substituted with halogen, lower alkoxy, lower alkyl, hydroxycarbony, carboxy, carboxy lower alkoxy, oxo and CN,
heteroaryl optionally substituted with halogen, lower alkoxy, lower alkyl, carboxy, hydroxyl, carboxy lower alkoxy, oxo, nitro and CN,
heterocycle optionally substituted with halogen, lower alkoxy, lower alkyl, hydroxycarbony, carboxy, carboxy lower alkoxy, oxo and CN,
cycloalkyl optionally substituted with halogen, lower alkoxy, lower alkyl, hydroxycarbony, carboxy, carboxy lower alkoxy, oxo and CN, and
cycloalkenyl optionally substituted with halogen, lower alkoxy, lower alkyl, hydroxycarbony, carboxy, carboxy lower alkoxy, oxo and CN;
R⁶ and R⁷ are each independently selected from the group consisting of H, (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ(CH₂)ₙ—COOH, (CH₂CH₂O)ₘ(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, —COR′, —SOR′, SO₂R′, Aryl-(CH₂)ₙ—COOH, heteroaryl-lower alkyl-CO-lower alkyl-NR′R″ wherein the lower alkyl may be substituted with OR′, and heteroaryl-(CH₂)ₙ-heterocycle wherein the heterocycle may optionally be substituted with lower alkyl, hydroxyl, COOR′ and COR′;
wherein R′ and R″ are as defined above for Y;
m, n and p are independently 0 to 6; and
q is 1 to 4;
or a pharmaceutically acceptable salt thereof.

2. The compound of claim 1 having the following stereochemistry

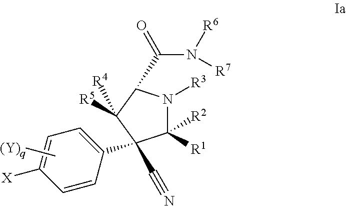

3. The compound of claim 2 wherein X is selected from the group consisting of H, F, Cl, Br, I, cyano, nitro, ethynyl, cyclopropyl, methyl, ethyl, isopropyl, vinyl and methoxy.

4. The compound of claim 2 wherein each Y is independently selected from the group consisting of H, F, Cl, Br, I, CN, OH, nitro, lower alkyl, cycloalkyl, lower alkoxy, lower alkenyl, lower cycloalkenyl and lower alkynyl, and q is 1 or 2.

5. The compound of claim 2 wherein one of R¹ and R² is H and the other is selected from the group consisting of
lower alkyl optionally substituted with OH or halogen, and
cycloalkyl optionally substituted with halogen, lower alkoxy, lower alkyl and carboxy.

6. The compound of claim 2 wherein R³ is selected from the group consisting of (CH₂)ₙ—R′, (CH₂)ₙ—COOR′, (CH₂)ₙ—CONR′R″, (CH₂)ₙ—OR′, (CH₂)ₙ—COR′, and R′ and R″ are each independently selected from
H,
lower alkyl, optionally substituted by hydroxy and amino,
aryl optionally substituted with lower alkyl, lower alkoxy, halogen and carboxy lower alkyl.

7. the compound of claim 2 wherein R⁴ is H and R⁵ is selected from the group consisting of aryl, aryl substituted with Cl or F, and heteroaryl optionally substituted with H, F, Cl, Br, I, CN, OH, nitro and lower alkyl.

8. The compound of claim 2 wherein R⁶ and R⁷ are independently selected from the group consisting of H, (CH₂)ₙ—R′, (CH₂)ₙ—NR′R″, (CH₂)ₙ—NR′COR″, (CH₂)ₙ—COOH, (CH₂)ₙ—COOR′, (CH₂)ₙ—OR′, (CH₂)ₙ—COR′, —COR′, —SOR′, SO₂R′, Aryl-COOR′, heteroaryl-lower alkyl-CO-lower alkyl-NR′R″ wherein the lower alkyl optionally is substituted with OR′, and R⁶ or R⁷ may be heteroaryl-(CH₂)ₙ-heterocycle wherein the heterocycle optionally may be substituted with lower alkyl, hydroxy, COOR′ and COR′.

9. The compound of claim 2 wherein m, n and p are 1 or 0 and q is 1.

10. A compound of formula Ia 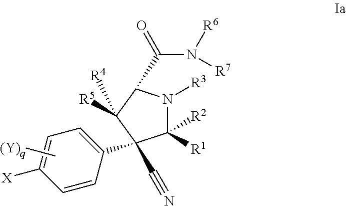
wherein
X is selected from the group consisting of H, F, Cl, Br, I, cyano, nitro, ethynyl, cyclopropyl, methyl, ethyl, isopropyl, vinyl and methoxy;
each Y is independently selected from the group consisting of H, F, Cl, Br, I, CN, OH, nitro, lower alkyl, cycloalkyl, lower alkoxy, lower alkenyl, lower cycloalkenyl and lower alkynyl;
one of R¹ and R² is H and the other is selected from the group consisting of
lower alkyl optionally substituted with OH or halogen, and
cycloalkyl optionally substituted with halogen, lower alkoxy, lower alkyl and carboxy;
R³ is selected from the group consisting of (CH₂)ₙ—R′, (CH₂)ₙ—COOR′, (CH₂)ₙ—CONR′R″, (CH₂)ₙ—OR′, (CH₂)ₙ—COR′,
or alternatively, R³ together with R¹ form a heterocycle that is optionally substituted with the group consisting of oxo and lower alkyl;
one of R⁴ and R⁵ is H and the other is selected from the group consisting of aryl, aryl substituted with Cl or F, and heteroaryl optionally substituted with H, F, Cl, Br, I, CN, OH, nitro and lower alkyl;
R⁶ and R⁷ are independently selected from the group consisting of H, (CH₂)ₙ—R′, (CH₂)ₙ—NR′R″, (CH₂)ₙ—NR′COR″, (CH₂)ₙ—COOH, (CH₂)ₙ—COOR′, (CH₂)ₙ—OR′, (CH₂)ₙ—COR′, —COR′, —SOR′ and SO₂R′, Aryl-COOR′—, heteroaryl-lower alkyl-CO-lower alkyl-NR′R″ wherein the lower alkyl optionally is substituted with OR′, and R⁶ or R⁷ may be heteroaryl-(CH₂)ₙ-heterocycle wherein the heterocycle optionally may be substituted with lower alkyl, hydroxy, COOR′ and COR′;
R′ and R″ are independently selected from
H,
lower alkyl optionally substituted with halogen, hydroxy, NH₂, NH-lower alkyl, N(lower-alkyl)₂, oxo, carboxy, carboxy lower alkyl, cycloalkyl and heteroaryl,
lower cycloalkyl optionally substituted with halogen, lower alkoxy, lower alkyl, carboxy, carboxy lower alkoxy, carboxy lower alkyl, oxo, CN, and NHSO₂-lower alkyl,
lower alkenyl optionally substituted with halogen, hydroxy, NH₂ and NH-lower alkyl and N(lower-alkyl)₂,
lower alkynyl optionally substituted with halogen, hydroxy, NH₂ and NH-lower alkyl and N(lower-alkyl)₂,
lower cycloalkenyl optionally substituted with halogen, lower alkoxy, lower alkyl, carboxy, carboxy lower alkoxy, oxo and CN,
aryl optionally substituted with halogen, lower alkoxy, hydroxy, lower alkyl, lower alkenyl, cycloalkyl, carboxy, carboxy lower alkoxy, carboxy lower alkyl, oxo, R¹ and CN, CONR′R²
hetereoaryl optionally substituted with halogen, lower alkoxy, lower alkyl, carbonyl, carboxy, carboxy lower alkyl, carboxy lower alkoxy, oxo, R¹ and CN, and
hetereocycle optionally substituted with halogen, lower alkoxy, carboxy, carboxy lower alkoxy, oxo CN, R′, SO₂-lower alkyl and lower alkyl that is optionally substituted with oxo; and
m, n and p are 0 or 1 and q is 1;
or a pharmaceutically acceptable salt thereof.

11. The compound of claim 10 wherein R′ and R″ are independently selected from
H,
lower alkyl optionally substituted by the group consisting of hydroxyl and amino, and
aryl optionally substituted with lower alkyl, lower alkoyl, halogen and carboxy-lower alkyl.

12. The compound of formula Ia 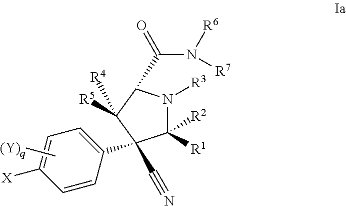
wherein
X is selected from the group consisting of F, Cl and Br;
Y is selected from the group consisting of H and F;
R¹ is lower alkyl;
R² is H;
R³ is selected from the group consisting of (CH₂)ₙ—R′, (CH₂)ₙ—COR′, (CH₂)ₙ—OR′ and (CH₂)ₙCONR′R″,
or alternatively, R³ together with R¹ form a heterocycle that is optionally substituted with the group consisting of oxo and lower alkyl;
R⁴ is H;
R⁵ is aryl optionally substituted with Cl or F;
R⁶ is H;
R⁷ is selected from the group
lower alkyl optionally substituted with cycloalkyl, heteroaryl, COOR′ and OR′,
heterocycle optionally substituted with R′, COR′ SO₂R′,
heteroaryl-lower alkyl-CO-lower alkyl-NR′R″, wherein the lower alkyl optionally is substituted with OR′,
aryl optionally substituted with R′, COOR′, OR′, halogen and CONR′R″, and
heteroaryl optionally substituted with OR′, COR′, R′, CO₂R′ and (CH₂)ₙ-heterocycle wherein the heterocycle optionally is substituted with OR′, COOR′ or COR′;
R′ is selected from the group consisting of
H,
lower alkyl optionally substituted with hydroxyl, carboxy, carboxy lower alkyl, cycloalkyl and heteroaryl,
lower cycloalkyl optionally substituted with carboxy-lower alkyl and NHSO₂-lower alkyl,
lower alkenyl,
lower alkynyl optionally substituted with halogen, hydroxyl, NH₂, NH-lower alkyl and N(lower alkyl)₂,
aryl optionally substituted with lower alkyl, lower alkoxy, F, Cl, lower alkyenyl, cycloalkyl, carboxy, carboxy lower alkyl and CONR¹R²,
heteroaryl optionally substituted with carboxy, carboxy-lower alkyl and R¹,
heterocycle optionally substituted with lower alkoxy, R¹, SO₂-lower alkyl and lower alkyl wherein the lower alkyl may be substituted with oxo;
m and n are independently 0 or 1; and
q is 1;
or a pharmaceutically acceptable salt thereof.

13. The compound of claim 2 wherein R¹ is 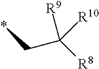
where R⁸ and R⁹ are both methyl, or alternatively, R⁸ and R⁹ together with the carbon to which they are attached form a ring selected from cyclopropyl, cyclobutyl, cyclopentyl and acyclohexyl;
R¹⁰ is (CH₂)ₘ—R¹¹, where m is 0, 1 or 2;
R^{H} is selected from the group consisting of hydrogen, hydroxyl, lower alkyl, lower alkoxy, aryl, hetereoaryl, and hetereocycle;
R² is H;
R³ is selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, wherein R′ and R″ are as defined above;
R⁴ is H;
R⁵ is a substituted phenyl selected from 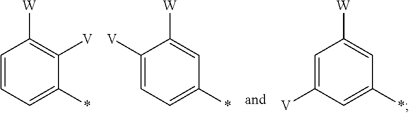
W is F, Cl or Br;
V is H or F;
one of R₆ and R₇ is hydrogen and the other is (CH₂)ₙ—R′;
n is 0 or 1; and
R′ is selected from the group consisting of
H,
lower alkyl optionally substituted with hydroxyl, carboxy, carboxy lower alkyl, cycloalkyl and heteroaryl,
lower cycloalkyl optionally substituted with carboxy-lower alkyl and NHSO₂-lower alkyl,
lower alkenyl,
lower alkynyl optionally substituted with halogen, hydroxyl, NH₂, NH-lower alkyl and N(lower alkyl)₂,
aryl optionally substituted with lower alkyl, lower alkoxy, F, Cl, lower alkyenyl, cycloalkyl, carboxy, carboxy lower alkyl and CONR¹R²,
heteroaryl optionally substituted with carboxy, carboxy-lower alkyl and R¹, and
heterocycle optionally substituted with lower alkoxy, R¹, SO₂-lower alkyl and lower alkyl wherein the lower alkyl may be substituted with oxo;
or a pharmaceutically acceptable salt thereof.

14. The compound of claim 2 selected from the group consisting of
rac-(2R,3R,4R,5S)-3-(3-chloro-phenyl)-4-(4-chloro-phenyl)-4-cyano-5-(2,2-dimethyl-propyl)-1-ethyl-pyrrolidine-2-carboxylic acid,
rac-(2R,3S,4R,5S)-3-(3-chloro-2-fluoro-phenyl)-4-(4-chloro-2-fluoro-phenyl)-4-cyano-5-methyl-1-propionyl-pyrrolidine-2-carboxylic acid (3,4-dihydroxy-butyl)-amide,
rac-(2R,3S,4R,5S)-3-(3-chloro-2-fluoro-phenyl)-4-(4-chloro-2-fluoro-phenyl)-4-cyano-1-(2-ethoxy-benzoyl)-5-methyl-pyrrolidine-2-carboxylic acid (3,4-dihydroxy-butyl)-amide,
rac-(2R,3S,4R,5S)-3-(3-chloro-2-fluoro-phenyl)-4-(4-chloro-2-fluoro-phenyl)-4-cyano-1-(3-ethoxy-benzoyl)-5-methyl-pyrrolidine-2-carboxylic acid (3,4-dihydroxy-butyl)-amide,
rac-(2R,3S,4R,5S)-3-(3-chloro-2-fluoro-phenyl)-4-(4-chloro-2-fluoro-phenyl)-4-cyano-1-(3-methyl-benzoyl)-5-methyl-pyrrolidine-2-carboxylic acid (3,4-dihydroxy-butyl)-amide
rac-(1R,2S,3R,7aS)-1-(4-chloro-2-fluoro-phenyl)-2-(3-chloro-2-fluoro-phenyl)-1-cyano-6,6-dimethyl-5-oxo-hexahydro-pyrrolizine-3-carboxylic acid ((S)-3,4-dihydroxy-butyl)-amide,
rac-(2R,3S,4R,5S)-3-(3-chloro-2-fluoro-phenyl)-4-(4-chloro-2-fluoro-phenyl)-4-cyano-5-(2,2-dimethyl-propyl)-1-formyl-pyrrolidine-2-carboxylic acid ((S)-3,4-dihydroxy-butyl)-amide,
rac-(1R,2S,3R,7aS)-1-(4-chloro-2-fluoro-phenyl)-2-(3-chloro-2-fluoro-phenyl)-1-cyano-6,6-dimethyl-hexahydro-pyrrolizine-3-carboxylic acid ((S)-3,4-dihydroxy-butyl)-amide, and
rac-(2R,3S,4R,5S)-3-(3-chloro-2-fluoro-phenyl)-4-(4-chloro-2-fluoro-phenyl)-4-cyano-1-(2-methyl-benzoyl)-5-methyl-pyrrolidine-2-carboxylic acid (3,4-dihydroxy-butyl)-amide,
and the pharmaceutically acceptable salts of the foregoing compounds.

15. The compound of claim 2 selected from the group consisting of
rac-(2R,3S,4R,5R)-1-acetyl-3-(3-chloro-2-fluoro-phenyl)-4-(4-chloro-2-fluoro-phenyl)-4-cyano-5-methyl-pyrrolidine-2-carboxylic acid (1-acetyl-piperidin-4-yl)-amide,
rac-(2R,3S,4R,5S)-1-acetyl-3-(3-chloro-2-fluoro-phenyl)-4-(4-chloro-2-fluoro-phenyl)-4-cyano-5-methyl-pyrrolidine-2-carboxylic acid (1-acetyl-piperidin-4-yl)-amide,
rac-(2R,3S,4R)-3-(3-chloro-2-fluoro-phenyl)-4-(4-chloro-2-fluoro-phenyl)-4-cyano-1-(3-methyl-benzyl)-5-methyl-pyrrolidine-2-carboxylic acid (1-acetyl-piperidin-4-yl)-amide,
rac-(2R,3S,4R)-3-(3-chloro-2-fluoro-phenyl)-4-(4-chloro-2-fluoro-phenyl)-4-cyano-5-methyl-1-(3-methyl-benzyl)-pyrrolidine-2-carboxylic acid (1-methanesulfonyl-piperidin-4-yl)-amide,
rac-(2R,3S,4R)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid (1-acetyl-piperidin-4-yl)-amide
rac-(2R,3S,4R,5S)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid (1-acetyl-piperidin-4-yl)-amide,
rac-(2R,3S,4R,5R)-3-(3-chloro-2-fluoro-phenyl)-4-(4-chloro-2-fluoro-phenyl)-4-cyano-1-(2-fluoro-benzyl)-5-methyl-pyrrolidine-2-carboxylic acid (1-acetyl-piperidin-4-yl)-amide,
rac-(1R,2S,3R,8aS)-2-(3-chloro-2-fluoro-phenyl)-1-(4-chloro-2-fluoro-phenyl)-1-cyano-7,7-dimethyl-octahydro-indolizine-3-carboxylic acid ((S)-3,4-dihydroxy-butyl)-amide,
rac-(2R,3S,4R,5R)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid (1-methanesulfonyl-piperidin-4-yl)-amide, and
rac-(2R,3S,4R,5S)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid (1-methanesulfonyl-piperidin-4-yl)-amide,
and the pharmaceutically acceptable salts of the foregoing compounds.

16. The compound of claim 2 selected from the group consisting of
(2R,3S,4R)-3-(3-chloro-2-fluoro-phenyl)-4-(4-chloro-2-fluoro-phenyl)-4-cyano-5-methyl-1-(3-methyl-but-2-enyl)-pyrrolidine-2-carboxylic acid (4-methanesulfonylamino-cyclohexyl)-amide,
rac-(2R,3S,4R)-3-(3-chloro-2-fluoro-phenyl)-4-(4-chloro-2-fluoro-phenyl)-4-cyano-1-(3-methoxy-benzyl)-5-methyl-pyrrolidine-2-carboxylic acid (1-acetyl-piperidin-4-yl)-amide,
rac-(2R,3S,4R)-3-(3-chloro-2-fluoro-phenyl)-4-(4-chloro-2-fluoro-phenyl)-4-cyano-1-(2-chloro-benzyl)-5-methyl-pyrrolidine-2-carboxylic acid (1-acetyl-piperidin-4-yl)-amide,
rac-(2R,3S,4R,5S)-1-butyl-3-(3-chloro-2-fluoro-phenyl)-4-(4-chloro-2-fluoro-phenyl)-4-cyano-5-(2,2-dimethyl-propyl)-pyrrolidine-2-carboxylic acid [1-(1-butyl-4-hydroxy-piperidin-4-ylmethyl)-1H-pyrazol-3-yl]-amide,
rac-propionic acid 4-(3-{[(2R,3S,4R,5S)-3-(3-chloro-2-fluoro-phenyl)-4-(4-chloro-2-fluoro-phenyl)-4-cyano-5-(2,2-dimethyl-propyl)-1-propionyl-pyrrolidine-2-carbonyl]-amino}-pyrazol-1-ylmethyl)-1-propionyl-piperidin-4-yl ester,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-benzoic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-fluoro-benzoic acid trifluoroacetate salt,
chiral 2-(4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]amino}-phenyl)-2-methyl-propionic acid, and
rac 4-({(2R,3S,4R,5S)-3-(3-chloro-2-fluoro-phenyl)-4-(4-chloro-2-fluoro-phenyl)-4-cyano-5-(2,2-dimethyl-propyl)-1-[2-(2-fluoro-phenyl)-ethyl]-pyrrolidine-2-carbonyl}-amino)-benzoic acid methyl ester,
and the pharmaceutically acceptable salts of the foregoing compounds.

17. The compound of claim 2 selected from the group consisting of
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-benzoic acid methyl ester,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-propyl-pyrrolidine-2-carbonyl]-amino}-benzoic acid, chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-isobutyl-pyrrolidine-2-carbonyl]-amino}-benzoic acid,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}benzoic acid,
rac-4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-benzoic acid,
rac-4-{[(2R,3S,4R,5S)-3-(3-chloro-2-fluoro-phenyl)-4-(4-chloro-2-fluoro-phenyl)-4-cyano-1-cyclobutylmethyl-5-(2,2-dimethyl-propyl)-pyrrolidine-2-carbonyl]-amino}-benzoic acid,
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-benzoic acid,
rac-4-{[(2R,3S,4R,5S)-3-(3-chloro-2-fluoro-phenyl)-4-(4-chloro-2-fluoro-phenyl)-4-cyano-1-(2-cyclohexyl-ethyl)-5-(2,2-dimethyl-propyl)-pyrrolidine-2-carbonyl]-amino}-benzoic acid,
chiral 4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-2-methoxy-benzoic acid,
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid,
and the pharmaceutically acceptable salts of the foregoing compounds.

18. The compound of claim 2 selected from the group consisting of
rac-4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-3-methoxy-benzoic acid,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-2-methoxy-benzoic acid,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-methoxy-benzoic acid,
rac 4-{[(2R,3S,4R,5S)-1-butyl-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid,
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-3-methoxy-benzoic acid,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(3-methyl-butyl)-pyrrolidine-2-carbonyl]-amino}-2-methoxy-benzoic acid,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-pentyl-pyrrolidine-2-carbonyl]-amino}-2-methoxy-benzoic acid,
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-pent-4-enyl-pyrrolidine-2-carbonyl]-amino}-2-methoxy-benzoic acid,
rac-4-{[(2R,3S,4R,5S)-3-(3-chloro-2-fluoro-phenyl)-4-(4-chloro-2-fluoro-phenyl)-4-cyano-1-(2-cyclohexyl-ethyl)-5-(2,2-dimethyl-propyl)-pyrrolidine-2-carbonyl]-amino}-2-methoxy-benzoic acid trifluoroacetate salt,
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-propyl-pyrrolidine-2-carbonyl]-amino}-2-methoxy-benzoic acid, and
rac-(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (4-carbamoyl-2-methoxy-phenyl)-amide, and
the pharmaceutically acceptable salts of the foregoing compounds.

19. The compound of claim 2 selected from the group consisting of
rac-(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (2-methoxy-4-methylcarbamoyl-phenyl)-amide,
rac-(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carboxylic acid {1-[2-((R)-3-dimethylamino-2-hydroxy-propoxy)-2-methyl-propyl]-1H-pyrazol-3-yl}-amide,
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-(5-hydroxy-pentyl)-pyrrolidine-2-carbonyl]-amino}-benzoic acid,
chiral 5-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2H-[1,2,4]triazole-3-carboxylic acid methyl ester,
chiral 2-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3H-imidazole-4-carboxylic acid trifluoracetate salt,
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-(5-hydroxy-pentyl)-pyrrolidine-2-carbonyl]-amino}-3-methoxy-benzoic acid,
chiral (2R,3S,4R,5S)-4-(4-chloro-2-fluoro-phenyl)-3-(3-chloro-2-fluoro-phenyl)-4-cyano-1-cyclopropylmethyl-5-(2,2-dimethyl-propyl)-pyrrolidine-2-carboxylic acid ethylamide,
chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid [1-(2-hydroxy-2-methyl-propyl)-1H-pyrazol-3-yl]-amide,
(R)-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-cyclopropyl-acetic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(2-ethoxycarbonyl-cyclopropylmethyl)-pyrrolidine-2-carbonyl]-amino}-3-methoxy-benzoic acid methyl ester,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-prop-2-ynyl-pyrrolidine-2-carbonyl]-amino}-3-methoxy-benzoic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(ethoxycarbonylmethyl-carbamoyl)-pyrrolidine-2-carbonyl]-amino}-3-methoxy-benzoic acid,
chiral (2S,3R,4S,5R)-4-(3-chloro-2-fluoro-phenyl)-3-(4-chloro-2-fluoro-phenyl)-2-(2,2-dimethyl-propyl)-5-((R)-2-hydroxymethyl-pyrrolidine-1-carbonyl)-1-methyl-pyrrolidine-3-carbonitrile,
chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid [(S)-2-hydroxy-1-(1H-imidazol-4-ylmethyl)-ethyl]-amide trifluoroacetate salt,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-1-ethyl-1H-pyrrole-2-carboxylic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1S,2S)-2-methoxycarbonyl-cyclopropylmethyl)-pyrrolidine-2-carbonyl]-amino}-3-methoxy-benzoic acid methyl ester,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1R,2R)-2-methoxycarbonyl-cyclopropylmethyl)-pyrrolidine-2-carbonyl]-amino}-3-methoxy-benzoic acid methyl ester,
4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1S,2S)-2-ethoxycarbonyl-cyclopropylmethyl)-pyrrolidine-2-carbonyl]-amino}-3-methoxy-benzoic acid,
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-((1R,2R)-2-ethoxycarbonyl-cyclopropylmethyl)-pyrrolidine-2-carbonyl]-amino}-3-methoxy-benzoic acid, and
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-(5-hydroxy-pentyl)-pyrrolidine-2-carbonyl]-amino}-2-methoxy-benzoic acid,
and the pharmaceutically acceptable salts of the foregoing compounds.

20. A compound selected from the group consisting of
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-benzoic acid;
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-fluoro-benzoic acid trifluoroacetate salt;
chiral 2-(4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]amino}-phenyl)-2-methyl-propionic acid;
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid;
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-isobutyl-pyrrolidine-2-carbonyl]-amino}-benzoic acid;
rac 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}benzoic acid;
rac-4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-benzoic acid;
rac-4-{[(2R,3S,4R,5S)-3-(3-chloro-2-fluoro-phenyl)-4-(4-chloro-2-fluoro-phenyl)-4-cyano-1-(2-cyclohexyl-ethyl)-5-(2,2-dimethyl-propyl)-pyrrolidine-2-carbonyl]-amino}-benzoic acid;
chiral 4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-2-methoxy-benzoic acid;
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3-methoxy-benzoic acid;
rac-4-{[(2R,3S,4R,5S)-3-(3-chloro-2-fluoro-phenyl)-4-(4-chloro-2-fluoro-phenyl)-4-cyano-1-cyclopropylmethyl-5-(2,2-dimethyl-propyl)-pyrrolidine-2-carbonyl]-amino}-3-methoxy-benzoic acid;
racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-2-methoxy-benzoic acid;
racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2-methoxy-benzoic acid;
racemic 4-{[(2R,3S,4R,5S)-1-butyl-3-(3-chloro-2-fluoro-phenyl)-4-(4-chloro-2-fluoro-phenyl)-4-cyano-5-(2,2-dimethyl-propyl)-pyrrolidine-2-carbonyl]-amino}-2-methoxy-benzoic acid;
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-ethyl-pyrrolidine-2-carbonyl]-amino}-3-methoxy-benzoic acid;
racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(3-methyl-butyl)-pyrrolidine-2-carbonyl]-amino}-2-methoxy-benzoic acid;
racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-pent-4-enyl-pyrrolidine-2-carbonyl]-amino}-2-methoxy-benzoic acid;
racemic 4-{[(2R,3S,4R,5S)-3-(3-chloro-2-fluoro-phenyl)-4-(4-chloro-2-fluoro-phenyl)-4-cyano-1-(2-cyclohexyl-ethyl)-5-(2,2-dimethyl-propyl)-pyrrolidine-2-carbonyl]-amino}-2-methoxy-benzoic acid trifluoroacetate salt;
racemic 4-{[(2R,3S,4R,5S)-3-(3-chloro-2-fluoro-phenyl)-4-(4-chloro-2-fluoro-phenyl)-4-cyano-5-(2,2-dimethyl-propyl)-1-propyl-pyrrolidine-2-carbonyl]-amino}-2-methoxy-benzoic acid;
rac-(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (4-carbamoyl-2-methoxy-phenyl)-amide;
rac-(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid (2-methoxy-4-methylcarbamoyl-phenyl)-amide;
chiral 5-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-2H-[1,2,4]triazole-3-carboxylic acid methyl ester;
chiral 2-{[(2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carbonyl]-amino}-3H-imidazole-4-carboxylic acid trifluoracetate salt;
rac-4-{[(2R,3S,4R,5S)-3-(3-chloro-2-fluoro-phenyl)-4-(4-chloro-2-fluoro-phenyl)-4-cyano-5-(2,2-dimethyl-propyl)-1-(5-hydroxy-pentyl)-pyrrolidine-2-carbonyl]-amino}-3-methoxy-benzoic acid;
chiral (2R,3S,4R,5S)-3-(3-chloro-2-fluoro-phenyl)-4-(4-chloro-2-fluoro-phenyl)-4-cyano-5-(2,2-dimethyl-propyl)-1-methyl-pyrrolidine-2-carboxylic acid [1-(2-hydroxy-2-methyl-propyl)-1H-pyrazol-3-yl]-amide;
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-(2-ethoxycarbonyl-cyclopropylmethyl)-pyrrolidine-2-carbonyl]-amino}-3-methoxy-benzoic acid methyl ester; and
chiral 4-{[(2R,3S,4R,5S)-3-(3-chloro-2-fluoro-phenyl)-4-(4-chloro-2-fluoro-phenyl)-4-cyano-5-(2,2-dimethyl-propyl)-1-prop-2-ynyl-pyrrolidine-2-carbonyl]-amino}-3-methoxy-benzoic acid,
and the pharmaceutically acceptable salts of the foregoing compounds.

21. A pharmaceutical composition comprising a compound of claim 2, or a pharmaceutically acceptable salt thereof, as an active ingredient and a pharmaceutically acceptable carrier or excipient.
